SEQUENCE LISTING (1 sequence)

SEQ ID NO: 1 - protein, 897 aa, Homo sapiens
MERAGATSRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

(19) United States
(12) Patent Application Publication
(10) Pub. No.: US 2026/0216344 A1
(43) Pub. Date: Jul. 30, 2026

(54) ALC1 Inhibitors and Synergy with PARPi

(71) Applicant: Eisbach Bio GmbH, Martinsried (DE)

(72) Inventors: William M. Menzer, München (DE); Gunnar Knobloch, Planegg (DE); Corinna Lieleg, Garching (DE); Adrian Schomburg, Gräfelfing (DE); Andreas Ladurner, Gräfelfing (DE); Peter Sennhenn, München (DE)

(21) Appl. No.: 19/284,162

(22) Filed: Jul. 29, 2025

Related U.S. Application Data
(63) Continuation of application No. 18/039,740, filed on Dec. 3, 2021.

(30) Foreign Application Priority Data
Dec. 3, 2020 (EP) 20211730.5

(51) Int. Cl. A61K 47/54; A61K 31/454; A61K 31/502; A61K 31/5025; A61K 45/06; A61K 47/55; A61P 35/00

(52) U.S. Cl. CPC A61K 47/545; A61K 31/454; A61K 31/502; A61K 31/5025; A61K 45/06; A61K 47/55; A61P 35/00

(57) ABSTRACT

The present invention relates to small molecule compounds that allosterically inhibit ALC1 (CHD1L) and which induce the trapping of PARP1, PARP2 and/or PARP3 on chromatin or at DNA damage sites. Disruption of the chromatin remodeling forces of ALC1 through these agents enables a highly selective therapy for targeting the DNA damage functions of PARP enzymes in several proliferative diseases, notably BRCA-deficient cancers. Via inhibition of the enzymatic activity, the compounds engage the synthetic lethality between BRCA1/2 and ALC1. By trapping PARP enzymes, inhibitors of ALC1 potentiate the cancer cell killing properties of PARP inhibitors, enable therapeutic approaches where ALC1 is amplified as an oncogene, therapeutically make it possible to overcome PARP inhibitor resistance mechanisms and enable an alternative approach to the treatment of germline or acquired BRCA1/BRCA2 deficiency, including tumors defined by “BRCAness” or other changes in DNA repair networks.

### CROSS REFERENCE TO RELATED APPLICATION(S)

This application is a continuation of U.S. application Ser. No. 18/039,740, filed on Jun. 1, 2023, which is a U.S. National Phase of International Application No. PCT/EP2021/084089, filed on Dec. 3, 2021, which claims priority to European Patent Application No. 20211730.5, filed Dec. 3, 2020 all of which are incorporated by reference herein in their entirety.

### SEQUENCE LISTING STATEMENT

A computer readable form of the Sequence Listing is filed with this application by electronic submission and is incorporated into this application by reference in its entirety. The Sequence Listing is contained in the file created on Jul. 23, 2025 having the file name “23-0775-WO-US-CON.xml” and is 2,763 bytes in size.

The present invention relates to small molecule compounds that allosterically inhibit ALC1 (CHD1L) and which induce the trapping of PARP1, PARP2 and/or PARP3 on chromatin or at DNA damage sites. Disruption of the chromatin remodeling forces of ALC1 through these agents enables a highly selective therapy for targeting the DNA damage functions of PARP enzymes in several proliferative diseases, notably BRCA-deficient cancers. Via inhibition of the enzymatic activity, the compounds engage the synthetic lethality between mutations in HRD pathways, including BRCA1/2 and ALC1. By trapping PARP enzymes, inhibitors of ALC1 potentiate the cancer cell killing properties of PARP inhibitors, enable therapeutic approaches where ALC1 is amplified as an oncogene, therapeutically make it possible to overcome PARP inhibitor resistance mechanisms and enable an alternative approach to the treatment of germline or acquired BRCA1/BRCA2 deficiency, including tumors defined by “BRCAness” or other changes in DNA repair networks.

### BACKGROUND OF THE INVENTION

For recognition of single-strand and double-strand breaks (SSBs/DSBs), the nuclear Poly-ADP-ribose polymerase (PARP) enzymes are early key factors in the DNA damage response (DDR). Using the metabolite NAD⁺, PARP-1 and -2 add poly-ADP-ribose (PAR) chains to chromatin components and to factors belonging to the DDR, while PARP-3 targets chromatin components via mono-ADP-ribosylation. PARPs get recruited to DNA lesions by recognizing specifically altered, DNA-damage induced structures, which turns on their PARylation activity, which in turn regulates their and the activity of other DDR and chromatin proteins, facilitating the DDR (Ray Chaudhuri and Nussenzweig, 2017).

This catalytic activity can be inhibited by NAD⁺ analogues and has become of particular interest and clinically useful in genetically-defined cancers. Notably, by targeting synthetic lethality in the context of BRCA1 or BRCA2 deficiency, so-called PARP inhibitors (PARPi) are used to treat homologous-repair (HR)-deficient and other cancers. This is thought to occur by lowering PARP activity and/or by biochemically “trapping” of PARP-1/2/3 on chromatin (Murai et al., 2012, 2014). While “trapping” remains molecularly ill-defined, the term defines an enhanced recruitment, association and/or retention of PARP-1/2/3 enzymes on damaged chromatin, typically induced by treatment of PARP-1 enzyme with PARP inhibitors (PARPi), or a decrease in the release of PARP-1/2/3 enzymes following their initial recruitment, which leads to a prolonged retention. This biochemically manifests itself in an enhanced steady-state association/retention/binding (“trapping”) of the PARP enzymes with damaged genome regions/loci.

Due to the clinical advent of PARPi, PARP-1 has emerged as a powerful target for an increasing list of cancers, including in combination with immuno-oncology therapies, such as the current Lynparza/Keytruda trials, to give all but one of many examples. Moreover, first-line PARPi therapies and applications in contexts outside of germline BRCA-1/2 mutations are becoming possible.

Importantly, clinical PARPi compounds all bind essentially the same location at the catalytic center of the active site by blocking the binding of the substrate NAD⁺, thus preventing poly(ADP-ribose) synthesis, largely by virtue of their structural similarity to nicotinamide, a moiety of the NAD⁺ nucleotide.

Yet, PARPi exhibit greatly different clinical efficacy in tumor killing and patient outcomes in the clinic. One fundamental difference in the action of these PARPi is that they promote highly distinct levels of PARP-1/-2 trapping on chromatin. It is currently generally thought that the most powerful and clinically effective PARPi trap PARP-1 at the site of a DNA break much more strongly than clinically less useful PARPi. Upon PARP trapping, DNA lesions become more cytotoxic, especially in mutant tumor cells with genetic or epigenetic deficiencies in the repair of DNA strand breaks, such as functionally HR-deficient BRCA-1/2 mutant tumor cells. Further, it is currently not clear what relative or dominant contributions are carried out by PARP-1 vs. PARP-2 vs. PARP-3 enzymes, since all enzymes are involved in sensing DNA strand breaks and recruit to DNA damage sites, while PARP-1 and PARP-2 both promote PARylation of chromatin factors, while all existing clinical PARPi molecules barely distinguish between the two related PAR polymerase enzymes PARP-1 and PARP-2. In turn, PARP trapping is thought to lead to DNA replication stress, genomic instability and cell death in cancer cells (Lord and Ashworth, 2012). Enhanced trapping of PARP1, for example, is thought to lead to an increased ability to kill cancer cells, especially cancers with defective DNA repair pathways (Zandarashvili et al., 2020). FIG. 1 shows the cytotoxic mechanism of PARP trapping via PARPi.

“PARP trapping” is thus described as an (enhanced) association of PARP-1 or PARP-2 or PARP-3 with chromatin in living cells. Using PARPi, the allosteric mechanism that contributes to binding of PARP-1 to DNA can be disrupted (Zandarashvili et al., 2020), with some PARPi contributing to retention and others facilitating pro-release mechanisms based on in vitro PARP-1, PARPi and DNA interactions (Zandarashvili et al., 2020). The different mechanisms of PARPi on PARP trapping are shown in FIG. 3. U2OS cells treated with talazoparib show an enhanced retention of GFP-tagged PARP2 at induced DNA lesions, whereas cells treated with veliparib reveal overall less PARP2 recruitment to the DNA lesions.

Either PARP-1 or PARP-2 are necessary for a sufficient recruitment of SSB repair proteins to the damage site. Via PARylation, chromatin remodeling or histone-modifying enzymes are activated at DNA damage sites, which leads to changes in chromatin compaction (Luijsterburg et al., 2016; Mehrotra et al., 2011; Sellou et al., 2016; Smeenk et al., 2013; Timinszky et al., 2009).

Activation of PARP-1 and/or PARP-2 enzymes leads to the recruitment of many proteins, notably also specific chromatin remodeling enzymes, notably including the macrodomain-containing nucleosome remodeler ALC1 (CHD1L) (Ahel et al., 2009; Gottschalk et al., 2009; Lehmann et al., 2017; Singh et al., 2017). Macrodomains generally bind ADP-ribose, oligo-ADP-ribose and poly-ADP-ribose (Karras et al., 2005), thus proteins containing macrodomains respond and recruit to PARP activation sites on the genome, including during DNA damage and with relevance for cancer. Importantly, PAR or oligo-ADP-ribose binding to the macrodomains of ALC1 robustly turns on chromatin remodeling activity (Ahel et al., 2009; Gottschalk et al., 2009; Lehmann et al., 2017; Singh et al., 2017), revealing ALC1 as an allosterically-regulated chromatin remodeling enzyme, the first of its kind, and one of the very few enzymes whose catalytic activity is directly regulated by PAR. Additionally, ALC1 is a validated oncogene and is often genetically amplified together with PARP1 in BRCA1/2-deficient ovarian and breast cancer samples (see FIG. 2). ALC1 inhibitors, such as small molecules inhibiting the ATPase function and or nucleosome remodeling functions of ALC1, could potentiate the effect PARPi, lead to enhanced cancer cell killing and/or reduce off-target effects and thus lessen cellular toxicity in non-cancer cells. The fact that altering the expression level of ALC1 (in this instance by a CRISPR-based knockout) impacts the sensitivity of cancer cells to PARP inhibitors, also opens up the opportunity that altering the activity levels of ALC1 could overcome PARP inhibitor resistance, since removing ALC1 robustly potentiates the PARPi Olaparib to a level that may be sufficient to circumvent PARPi resistance, such as upon (but not limited to) reversion of the BRCA-deficiency status (e.g. by internal deletions or through loss of epigenetic BRCA1/2 gene silencing). Since the sensitivity of cancer cells to PARP inhibitors is generally accepted to (also) stem from the ability of PARPi to trap PARP1 on chromatin (and likely also trapping PARP2 enzymes—this has not been formally established in the field), we hypothesized that small molecule ALC1 inhibitors may mediate PARPi sensitization, bypass PARPi resistance and/or promote cancer cell killing through a direct or indirect impact on PARP1 and/or PARP2 trapping. Specifically, we hypothesized that inhibition of ALC1 with small molecules would enhance PARP1/2 trapping at DNA damage sites and thus, indirectly, promote DNA damage and cancer cell killing.

Recently, the first small molecule inhibitors targeting ALC1 (CHD1L) have been reported (Abbott et al., 2020). The publication revealed a role for ALC1 inhibitors in driving malignant colorectal cancer (CRC) due to its oncogenic function, specifically by impacting the Wnt/TCF-driven epithelial-to-mesenchymal transition (EMT) in CRC. Analysis with one specific ALC1 inhibitor further showed evidence for significant DNA damage, as measured by the DNA damage marker gammaH2AX. This led the authors to hypothesize that ALC1 may increase the efficacy of colorectal cancer standard-of-care DNA-damaging chemotherapies such as etoposide, which forms a ternary complex with DNA and the topoisomerase II enzyme, preventing re-ligation of the DNA strands following DNA replication, thus causing DNA strand breakage and cell death. Importantly, no suggestion, nor experimental evidence for an impact of ALC1 inhibitors on PARP-1/2/4 trapping was presented in this publication.

To analyze the mechanisms that cause the distinct trapping effects, the chromatin remodeler ALC1 (CHD1L) was hypothesized to regulate PARP retention. Containing a PAR-binding macrodomain, ALC1 regulates protein recruitment to DNA lesions. (Ahel et al., 2009; Gottschalk et al., 2009; Lehmann et al., 2017; Singh et al., 2017). As shown in FIGS. 5 and 6, inhibition of ALC1 via ALCi in U2OS cells led to specific retention of PARP-2 at DNA lesions.

Since ALC1 is upregulated in several tumors and is a validated oncogene in hepatocellular carcinoma (Cheng et al., 2013; Li et al., 2019; Su et al., 2014), inhibition of ALC1 via small molecule inhibitors leading to PARP-2 trapping could drive robust changes in DNA damage responses.

The present inventors hypothesized that manipulation of ALC1 activity via small molecules could be sufficient to bypass a low or high level of PARPi resistance. Thus, ALC1 and PARP-2 could be exploited to refine PARP-targeted therapies in oncology.

This hypothesis led to a part of the present invention that ALC1 manipulation via small molecule inhibitors impacts the response to DNA damage through PARP-1, PARP-2 and/or PARP-3 trapping, as well as the trapping of as-yet undescribed chromatin/DNA-damage associating PARP family members. These compounds, designed to inhibit the enzymatic activity of the ATP-dependent chromatin remodeler ALC1 (CHD1L), will potentiate accumulation of DNA damage and thus to mediate synthetic lethality upon BRCA deficiency in vitro, since PARP inhibition has been well documented to be synthetic lethal with loss of BRCA-1/2 tumor suppressor function based on preclinical and clinical evidence, suppress PARPi resistance mechanisms, open up ALC1 inhibition as a therapeutic approach for cancers that have intact HR pathways, and/or allow the targeting of ALC1-amplified tumors by disrupting ALC1's oncogene function such as HCC via promoting PARP-1/2/3 trapping. Also, since the ALC1 gene is a key mediator of PARP-chromatin rearrangements upon induced DNA damage (Sellou et al., 2016), small molecules targeting ALC1 activity may impact the nuclear, DNA-damage relevant functions of PARP-1/2/3 on chromatin rearrangements without impacting other roles of PARP-1/2/3 inside or outside of the cell's nucleus or without impacting non-DNA-damage induced PARP enzymes, which may result in a “second-generation PARPi” with reduced off-target effects and/or reduced side-effects.

The dynamics of PARP-1 and PARP-2 and PARP-3 enrichment at DNA damage sites can be visualized using live-cell imaging, as established for PARP-1 in the field (Ahel et al., 2009; Gottschalk et al., 2009). Wild-type PARP-1 and PARP-2 rapidly recruit to DNA lesions induced by laser-micro-irradiation. Via transient transfection of a GFP-tagged PARP-1 and PARP-2 in an immortalized cell line, we measured the impact of ALC1i on their recruitment to—and retention at DNA damage sites.

The present inventors were successful in identifying an allosteric binding pocket within the ATPase domain of ALC1 and used this information to model inhibitors of ALC1 activity. Given the above described relevance of ALC1 for various proliferative diseases, in particular those that are BRCA1/2 deficient. Thus the present invention provides a novel class of compounds to treat or ameliorate tumor diseases and in particular tumor diseases characterized by increased activity of ALC1, e.g. due to increased expression.

Furthermore, the present inventors determined that by using a combination of PARPi and an ALC1 inhibitor, preferably the allosteric ALC1 inhibitors of the present invention the effect of PARPis can surprisingly be enhanced. Thus, the present invention provides inter alia (i) an efficient therapy of tumors that are sensitive to PARPi, (ii) mediate PARPi sensitization, (iii) bypass PARPi resistance, (iv) allow the reduction of the amount of PARPi that is administered, and/or (v) promote cancer cell killing through a direct or indirect impact on PARP-1, PARP-2 and/or PARP-3 trapping.

### SUMMARY OF THE INVENTION

In a first aspect the present invention relates to an allosteric inhibitor of Chromodomain-helicase-DNA-binding protein 1-like (ALC1), wherein the inhibitor specifically binds to an allosteric binding pocket formed by an amino acid stretch spanning amino acid residues 101 to 219 of SEQ ID NO: 1.

In a further aspect the present invention relates to a compound of formula (I):

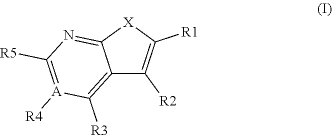

and isomers, salts, solvates, chemically protected forms, and prodrugs thereof, wherein:
X is N or S;
A is C or N;
R₁ is —CO—OR₆, —CO—R₇, or —CO—NR₆R_{A}, preferably R₁ is —CO—OR₆;
R₂ is —R₇, —NHR₈, —O—R₇, —C—O—R₇, Br, —C₃₋₈-cycloalkyl (preferably cyclopropyl), or —C₄₋₈-cycloalkenyl (preferably cyclohexenyl);
   or
   R₁ and R₂ together form a 5, 6 or 7 membered carbo- or heterocycle, optionally substituted, preferably with 1, 2 or 3 substituents independently selected from the group consisting of —OH, —NO₂, —CN, —Br, —Cl, —F, —I, —O—C₁₋₃-alkyl, ═O, [—O—CH₂—CH₂]—NH—CH(OH)—O-tBu;
R₃ is H, ═O, —OH, —O—R₇, —R₇, or —(CH₂)ₘ-L, wherein m is 0, 1 or 2, and L is a 5, 6 or 7 membered carbo- or heterocycle, optionally substituted, preferably with 1, 2, or 3 substituents independently selected from the group consisting of —OH, —NO₂, —CN, —Br, —Cl, —F, —I, —O—C₁₋₃-alkyl, -hydroxyC₁₋₃-alkyl and ═O;
R₄ is H or —C₁₋₃-alkyl, preferably H;
R₅ is —(CH₂)ₘ-L, or —(CH₂)ₘ—(CH═CH)-L, wherein m is 0, 1 or 2, preferably 0 or 1, and L is a 5, 6 or 7 membered carbo- or heterocycle, adamantyl, C₁₋₄-alkyl, or —N(CH₃)₂, optionally substituted, preferably with 1, 2, 3 or 4 substituents independently selected from the group consisting of —OH, —NO₂, —CN, —CO—OR₆, —Br, —Cl, —F, —I, —R₉, —O—R₉, ═O, and [—O—CH₂—CH₂]_{q}—NH-biotin with q being 1, 2, 3, or 4, or two adjacent substituents form a 5, 6 or 7 membered carbo- or heterocycle; or
   R₄ and R₅ together form a 5, 6, or 7 membered carbocycle, optionally substituted, preferably with 1, 2, or 3 substituents independently selected from the group consisting of —OH, —NO₂, —CN, —Br, —Cl, —F, —I, —O—R₉, —R₉, ═CH—R_{A}, and —CH₂—R_{A}, preferably R₄ and R₅ together form a C₅₋₇-cycloalkyl;
R₆ is H, —C₁₋₆-alkyl, —C₂₋₆-alkenyl, —C₂₋₆-alkynyl, optionally substituted, preferably R₆ is H;
R₇ is —C₁₋₃-alkyl, —C₂₋₃-alkenyl, —C₂₋₃-alkynyl, optionally substituted, preferably with 1, 2, or 3 substituents independently selected from the group consisting of —Br, —Cl, —F, —I, —CH₃, —OCH₃, or —SCH₃;
R₈ is H or C₁₋₆-alkyl, preferably H,
R₉ is —C₁₋₆-alkyl, —C₂₋₆-alkenyl, —C₂₋₆-alkynyl, —C₁₋₆-alkyl-aryl, or C₁₋₆-alkyl-heteroaryl (preferably isooxazole, thiazole, tetrazole, 1,2,4-thiadiazole, 1,2,3-thiadiazole, 1,2,5-thiadiazole, pyridine, 1,2,4-oxadiazole, pyrazine, or pyrazole), optionally substituted with 1, 2 or 3 substituents selected from the group consisting of —Br, —Cl, —F, —I, —NO₂, —CN, —CONH₂, —CONH—C₁₋₃-alkyl (preferably —CONH—CH₃), —NH—CO—C₁₋₃-alkyl (preferably —NH—CO—CH₃), —C₁₋₆-alkyl (preferably-CH₃, ethyl, propyl, t-butyl, or pentyl), —C₁₋₃-haloalkyl (preferably —CF₃, or —CHF₂), —O—CHF₂, —O—CF₃, carbocycle (preferably cyclopropyl, cyclohexyl or phenyl), —O-carbocycle (preferably phenoxy), heterocycle (preferably pyrazolyl), —CO-heterocycle (preferably-CO-(1-pyrrolidinyl)), —SO₂—CH₃, —SO₂—N(CH₃)₂, —O—C₁₋₄-alkyl (preferably —OCH₃), —O—C₁₋₃-alkyl-O—C₁₋₃-alkyl (preferably —O—CH₂—O—CH₃), —SCH₃, or when R₉ is —C₁₋₆-alkyl-aryl, then two adjacent substituents on the aryl moiety can form a 5, 6 or 7 membered carbo- or heterocycle, which is optionally substituted;
R_{A} is H, carbo- or heterocycle, optionally substituted, preferably with 1, 2 or 3 substituents independently selected from the group consisting of —OH, —NO₂, —CN, —Br, —Cl, —F, —I, —R₉, —O—R₇, —O—(CH₂)ₒ—R₉, —SO₂NH₂, and ═O, wherein o is 0 or 1.

In yet another aspect, the present invention relates to a compound of formula (I):

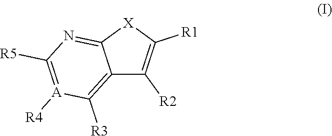

and isomers, salts, solvates, chemically protected forms, and prodrugs thereof, wherein:
X is N or S;
A is C or N;
R₁ is —CO—OR₆, —CO—R₇, or —CO—NR₆R_{A}, preferably R₁ is —CO—OR₆;
R₂ is —R₇, —NHR₈, —C—O—R₇;
or
R₁ and R₂ together form a 5, 6 or 7 membered carbo- or heterocycle, optionally substituted, preferably with 1, 2 or 3 substituents independently selected from the group consisting of —OH, —NO₂, —CN, —Br, —Cl, —F, —I, —O—C₁₋₃-alkyl, and ═O;
R₃ is H, ═O, —OH, —O—R₇, —R₇, or —(CH₂)ₘ-L, wherein m is 0, 1 or 2, and L is a 5, 6 or 7 membered carbo- or heterocycle, optionally substituted, preferably with 1, 2, or 3 substituents independently selected from the group consisting of —OH, —NO₂, —CN, —Br, —Cl, —F, —I, —O—C₁₋₃-alkyl, -hydroxyC₁₋₃-alkyl and ═O;
R₄ is H or —C₁₋₃-alkyl, preferably H;
R₅ is —(CH₂)ₘ-L, or —(CH₂)ₘ—(CH═CH)-L, wherein m is 0, 1 or 2, preferably 0 or 1, and L is a 5, 6 or 7 membered carbo- or heterocycle, or adamantyl, optionally substituted, preferably with 1, 2, 3 or 4 substituents independently selected from the group consisting of —OH, —NO₂, —CN, —CO—OR₆, —Br, —Cl, —F, —I, —R₉, —O—R₉, and ═O, or two adjacent substituents form a 5, 6 or 7 membered carbo- or heterocycle;
or
R₄ and R₅ together form a 5, 6, or 7 membered carbocycle, optionally substituted, preferably with 1, 2, or 3 substituents independently selected from the group consisting of —OH, —NO₂, —CN, —Br, —Cl, —F, —I, —O—R₉, —R₉, ═CH—R_{A}, preferably R₄ and R₅ together form a C₅₋₇-cycloalkyl;
R₆ is H, —C₁₋₆-alkyl, —C₂₋₆-alkenyl, —C₂₋₆-alkynyl, optionally substituted, preferably R₆ is H;
R₇ is —C₁₋₃-alkyl, —C₂₋₃-alkenyl, —C₂₋₃-alkynyl, optionally substituted, preferably with 1, 2, or 3 substituents independently selected from the group consisting of —Br, —Cl, —F, —I, —CH₃, —OCH₃, or —SCH₃;
R₈ is H or C₁₋₆-alkyl, preferably H,
R₉ is —C₁₋₆-alkyl, —C₂₋₆-alkenyl, —C₂₋₆-alkynyl, or —C₁₋₆-alkyl-aryl, optionally substituted with 1, 2 or 3 substituents selected from the group consisting of —Br, —Cl, —F, —I, —CH₃, —OCH₃, or —SCH₃
R_{A} is H, carbo- or heterocycle, optionally substituted, preferably with 1, 2 or 3 substituents independently selected from the group consisting of —OH, —NO₂, —CN, —Br, —Cl, —F, —I, —R₉, —O—R₇, —O—(CH₂)ₒ—R₉, —SO₂NH₂, and ═O, wherein o is 0 or 1.

In a second aspect the present invention relates to a bifunctional compound comprising the allosteric inhibitor of ALC1 the first or further aspect of the present invention and a compound which recruits E3 ubiquitin ligase to ALC1 (E3 recruiter), wherein the allosteric inhibitor of ALC1 and the E3 recruiter are covalently linked, optionally through a linker.

In a third aspect the present invention relates to a pharmaceutical composition comprising the allosteric inhibitor of ALC1 and pharmaceutically acceptable excipients.

In a fourth aspect the present invention relates to an ALC1 inhibitor (ALC1i) for use in treating or ameliorating a proliferative disease in a patient, wherein the method comprises the administration of said ALC1i and optionally the administration of a Poly(ADP-ribose)-Polymerase inhibitor (PARPi).

In a fifth aspect the present invention relates to a PARPi for use in treating or ameliorating a proliferative disease in a patient, wherein the method comprises the administration of said PARPi and the administration of said ALC1i.

In a sixth aspect the present invention relates to a kit of parts comprising separately packaged a PARPi and an ALC1i or a composition comprising a PARPi and an ALC1i, preferably with instructions for use to treat or ameliorate a proliferative disease.

### DESCRIPTION OF THE FIGURES

In the following, the content of the Figures comprised in this specification is described. In this context please also refer to the detailed description of the invention above and/or below.

FIG. 1: Cytotoxic mechanisms of PARPi on DNA repair pathways leading to PARP trapping. Upper pathway shows interference with DNA repair of single strand breaks (SSBs) via DNA replication fork damage leading to repair via the homologous recombination (HR) mechanism. Lower pathway shows trapping of PARP1/2 proteins on damaged DNA, leading to replication fork damage utilizing additional repair pathways including Fanconi pathway (FA), template switching (TS), ATM, FEN1 (replicative flap endonuclease) and DNA polymerase β (Murai et al., 2012, S. 5591).

FIG. 2: The chromatin remodeler ALC1 (CHD1L) is frequently co-amplified with the PARP1 gene in human ovarian and breast cancer samples. Genomic alterations of ALC1 (CHD1L), PARP1, PARP2, BRCA1, BRCA2 and the most closely related chromatin remodeler CHD1 among the genomes of 10792 breast, fallopian and ovarian cancer patients (OncoPrint analysis conducted on Aug. 12, 2020 at the cBioPortal-www.cbioportal.org). The percentage numbers indicate the percentage of alterations in a particular gene for all genomes where the specific gene has been profiled. Gene amplifications (black), deep gene deletions (dark grey) are highlighted. Compared to CHD1, ALC1 is often amplified together with PARP1 (ALC1-PARP1: Odd's ratio (OR)=1.581, q-value=<0.001; CHD1-PARP1: OR=−0.125, q-value=0.250). Neither ALC1, nor PARP1 exhibit a deep deletion or frequent mutation when the tumors exhibit a deep deletion or mutation in the BRCA1 or BRCA2 tumor suppressor genes (ALC1-BRCA1/2: OR=<−3/−2.178, q-value=<0.001; PARP1-BRCA1/2: OR=<−3, q-value=<0.001).

FIG. 3: GFP-PARP2 association at laser micro-irradiation sites in U20S cells. U2OS cells were treated with PARPi veliparib (10 μM) and talazoparib (100 nM) and transfected with GFP-PARP2. Grey signal indicates PARP2 in the nucleus. Bright lines show recruitment of PARP2 to laser microirradiated damage sites. Images were taken 1 minute and 15 minutes after irradiation. Treatment with talazoparib shows enhanced retention of PARP2 at induced damage sites (“PARP trapping”), whereas treatment with veliparib leads to less recruitment of PARP2 to the damage site.

FIG. 4: Schematic of live-cell PARP trapping assay. The cell nucleus microirradiated by a 355 nm wavelength laser to induce DNA damage (indicated by black bar, left cell). Cells are then imaged for a period of 15 minutes. Grey bar shows recruitment of PARP1/2 to the induced DNA damage site (middle cell). A) the signal of PARP2 reduces over time, indicating a decrease in PARP1/2 retention B) prolonged retention of PARP2, a live-cell imaging correlate showing molecular trapping of PARP2 at the induced DNA damage site.

FIG. 5: Relative recruitment of PARP2 to DNA damage site after treatment with ALCi-9. Wild-type U2OS cells were stably transfected with GFP-PARP2. Kinetics of GFP-PARP2 recruitment to and dissociation from DNA lesion was measured over 30 minutes in the presence and absence of compound ALCi-9. Treatment with ALCi-9 shows enhanced retention of PARP2 at DNA damage sites compared to DMSO. 9 nuclei were analyzed in 1 biological replicate. The data are shown as mean±S.E.M. normalized to pre-damaged GFP intensity at microirradiation sites.

FIG. 6: Relative recruitment of PARP2 to DNA damage site after treatment with ALCi-9. Wild-type U2OS cells were stably transfected with GFP-PARP2. Kinetics of GFP-PARP2 recruitment to and dissociation from DNA lesion was measured over 30 minutes in the presence and absence of compound ALCi-9. Upper nucleus was treated with DMSO, lower nucleus was treated with ALCi-9 (10 μM) for 1 h. Timepoint 0 min. shows nuclei before micro-irradiation, timepoint 1 min, and 30 min. show nuclei after irradiation. Treatment with ALCi-9 shows PARP2-trapping at DNA damage sites compared to DMSO.

FIG. 7: PARP2 trapping after co-treatment with ALCi-9 and PARPi veliparib. Wild-type U2OS cells were stably transfected with GFP-PARP2. Kinetics of GFP-PARP2 recruitment to and dissociation from DNA lesion was measured over 30 minutes in the presence and absence of veliparib, compound ALCi-9 or a combination of both. 4-11 nuclei were analysed in 1 biological replicate. The data are shown as mean±S.E.M. normalized to pre-damaged GFP intensity at micro-irradiation sites.

FIG. 8: Colony formation assay of BRCA positive and BRCA negative cells treated with ALCi. MDA-MB-231 cells (BRCA1/2 wildtype) and SUM-149-PT cells (BRCA1 negative) cells were seeded into 96-well plates and treated with titrations of different ALCi starting at 50 μM. The cells were cultured at 37° C., CO₂ 5% for 11 days, fixed with 10% TCA and stained with sulforhodamine staining to analyse cell survival. The data was normalized to DMSO controls indicating 100% survival. Inhibitor vs. response curves with variable slope (four parameters) were fitted using GraphPad Prism. Error bars are shown for two technical replicates.

FIG. 9: PARPi co-treatment colony formation assay of BRCA negative cells. SUM-149-PT cells (BRCA1 negative) cells were seeded into 96-well plates and treated with PARPi in different concentrations and titrations of different ALCi starting at 50 μM. The cells were cultured at 37° C., CO₂ 5% for 11 days, fixed with 10% TCA and stained with Sulforhodamine staining to analyze cell survival. Upper part of the figure shows survival curves of SUM-149-PT cells. Black curves indicate co-treatment of ALCi-x with PARPi-y. Treatment with ALCi-132 and ALCi-74 show enhanced cell killing compared to treatment with Veliparib only or ALCi only. Synergism of veliparib and ALCi-x are shown for certain concentrations in the bar-graphs. Here, control growth (%) under treatment with veliparib alone, treatment with ALCi-x alone and co-treatment are illustrated in grey.

FIG. 10: Structures and associated compound codes of ALC1 inhibitors.

FIG. 11: Nucleosome remodeling inhibition. IC₅₀s (μM) of ALC1 inhibitors in a FRET based nucleosome remodelling assay. IC₅₀ values are presented by the following symbols: +++: IC₅₀<25 μM; ++: IC₅₀=25-250 μM; +: IC₅₀>250 μM; and ‘−’: compounds is not active (for SAR purposes).

FIG. 12: Cell proliferation inhibition EC50s (μM) of ALC1 inhibitors in a 5 day cell proliferation assay with an SRB based readout. EC50 values are presented by the following symbols: +++: EC₅₀<10 μM; ++: EC₅₀=10-50 μM; +: EC₅₀>50 μM; and ‘−’: compounds is not active.

FIG. 13: Surface cutaway of the first lobe of the ALC1 helicase domain in the “front” (A) and “back” (B) orientations showing the newly identified allosteric binding pocket. The ATP binding site is denoted by a black circle.

FIG. 14: Surface cutaway of the first lobe of the ALC1 helicase domain with regions colored according to hydrophobicity in the “front” (A) and “back” (B) orientations. Darker colors are more hydrophilic.

FIG. 15: Surface cutaway of the first lobe of the ALC1 helicase domain surface of ALCi-22 with regions colored according to hydrophobicity in the “front” (A) and “back” (B) orientations. Darker colors are more hydrophilic.

FIG. 16: Surface cutaway of the first lobe of the ALC1 helicase domain with the Van der Waals surface of ligand ALCi-22 shown in white in the “front” (A) and “back” (B) orientations.

FIG. 17: Surface cutaway of the first lobe of the ALC1 helicase domain with the stick representation of the ligand ALCi-22 shown in white in the “front” (A) and “back” (B) orientations.

FIG. 18: Ligplot diagram of ALCi-22 bound to the allosteric site in the first lobe of the helicase domain of ALC1.

FIG. 19: Surface cutaway of the first lobe of the ALC1 helicase domain with ligand ALCi-22 shown in white in the “front” orientation with specific interactions with ASN165 and ARG135 shown with black dashed lines.

FIG. 20: PDB file for the novel allosteric pocket of ALC1. Structural coordinates of the key atoms of the amino acids at the surface of the allosteric binding pocket of human ALC1 comprised within the amino acids stretch spanning amino acids 101 to 219 of ALC1 according to SEQ ID NO: 1. The allosteric ALC1 inhibitors of the present invention interact with one or more of these key atoms in order to specifically bind to this pocket. The structure of the pocket of human ALC1 has been determined by homology modelling using swissmodel. The residues shown are the minimum set of amino acids required to reproduce docking results that were initially preformed on a larger ALC1 homology model generated using swissmodel. Specifically, the swissmodel model was used in initial docking experiments. This pocket was chosen due to its proximity to the ATP binding site and its novelty. These amino acids are required to form the pocket and allow for docking, and allow faster computation than when using the whole protein.

FIG. 21: A table containing the supplier used for each of the inhibitors

FIG. 22: A general synthesis scheme for inhibitors according to formula I

FIG. 23: A table containing the percentage inhibition of ALC1 in the FRET-based nucleosome sliding assay at a compound concentration of 250 μM for each of the inhibitors. For some compounds, the concentration of 250 μM had to be lowered due to solubility problems as indicated in the legend at the bottom of the table.

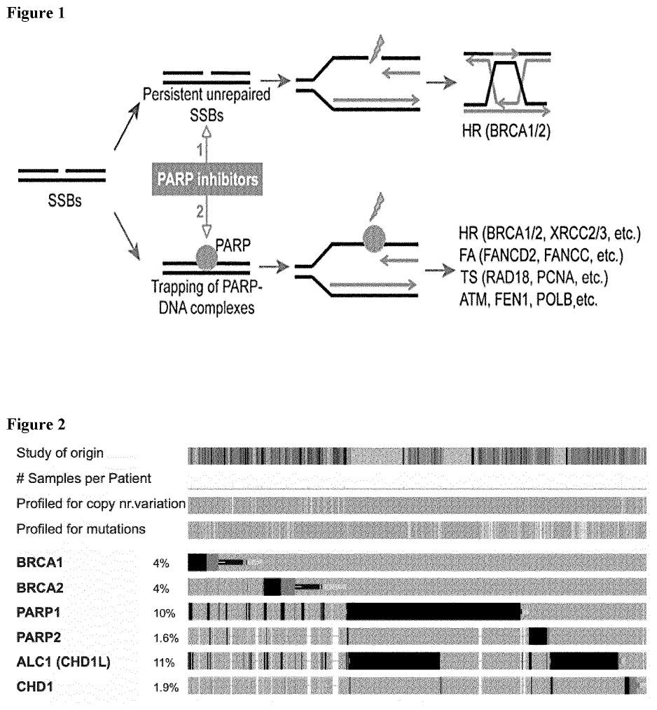

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

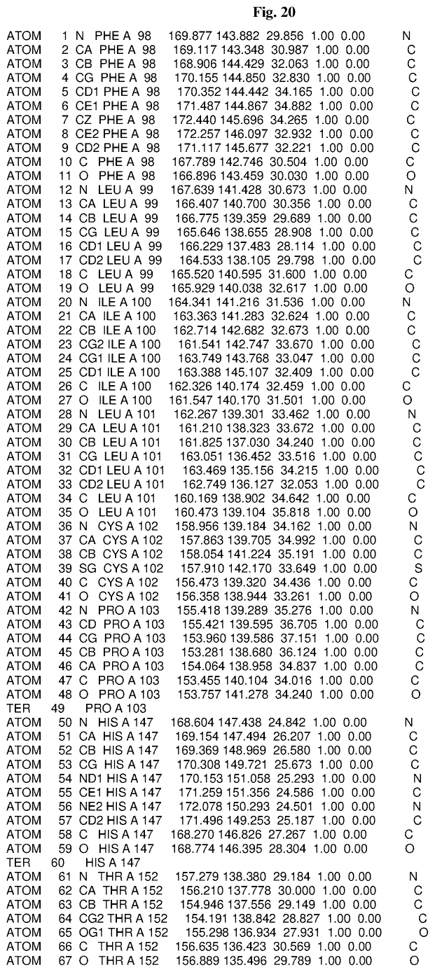

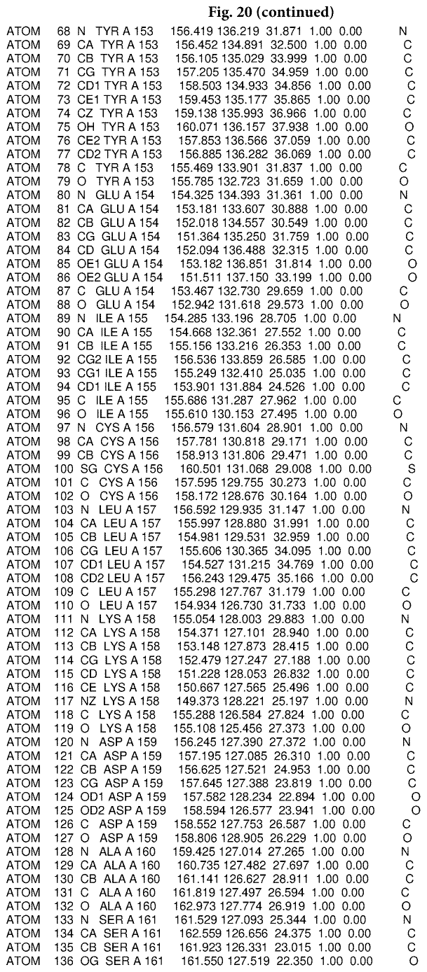

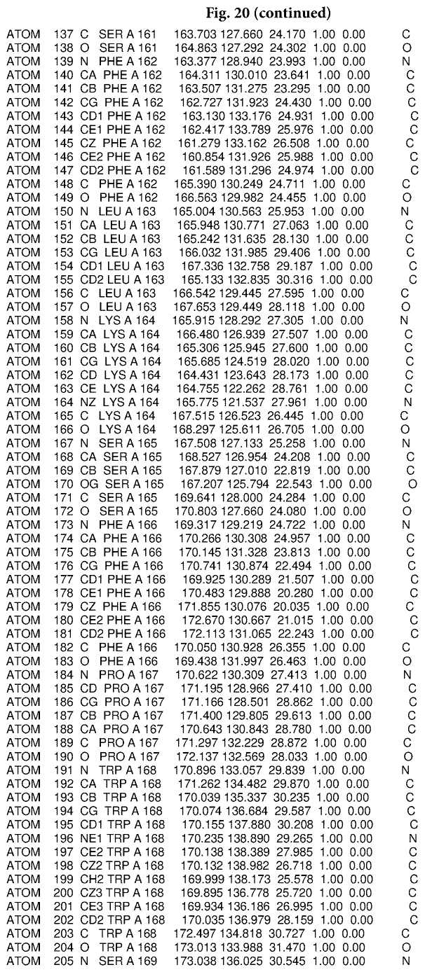

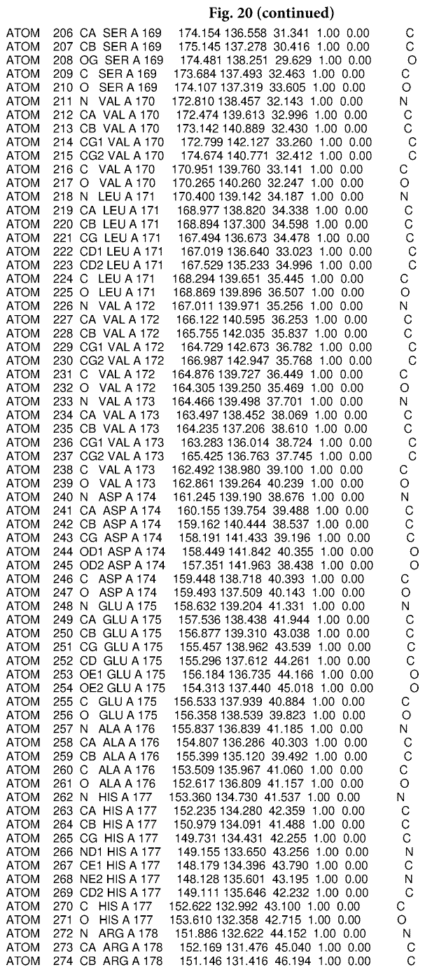

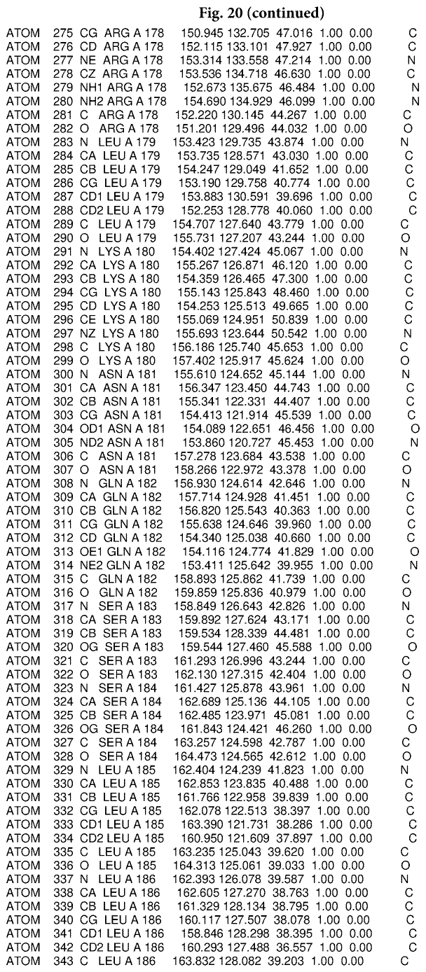

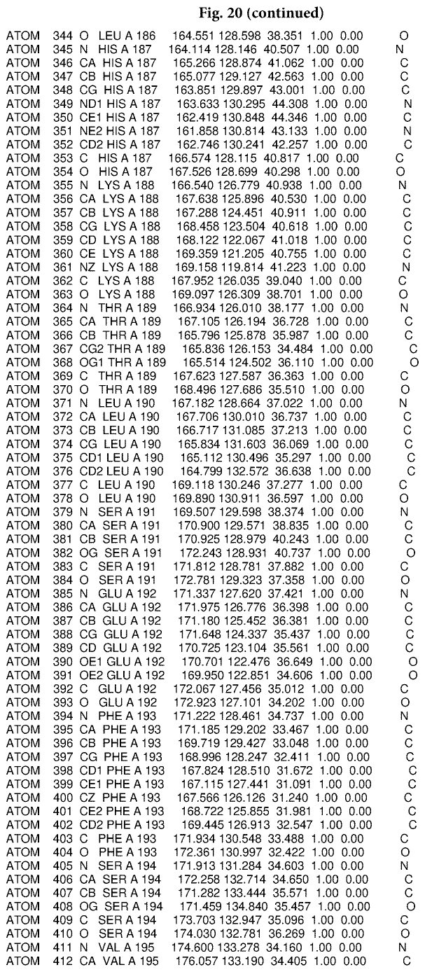

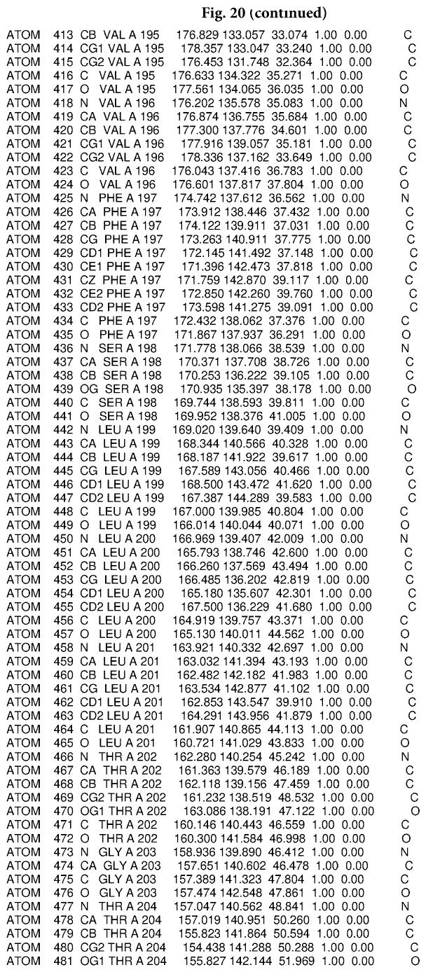

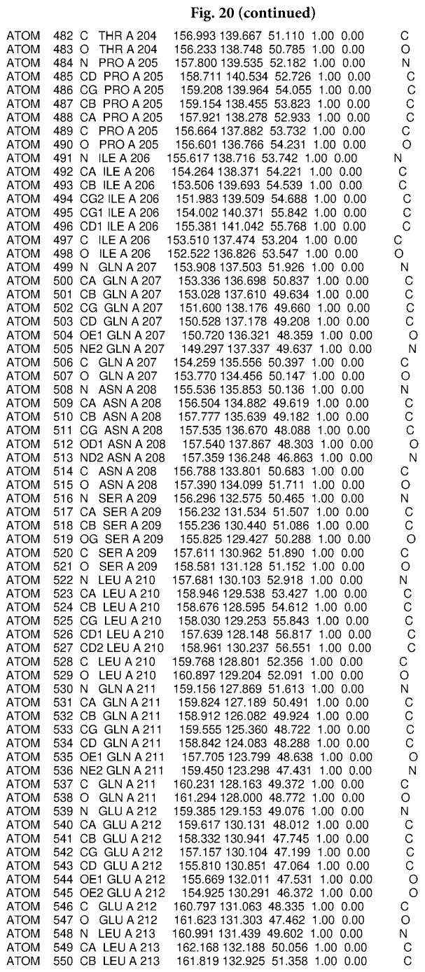

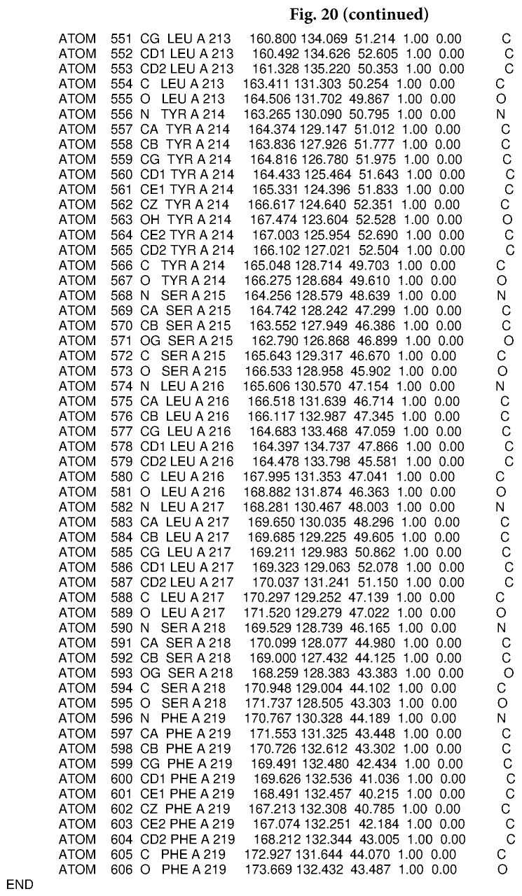

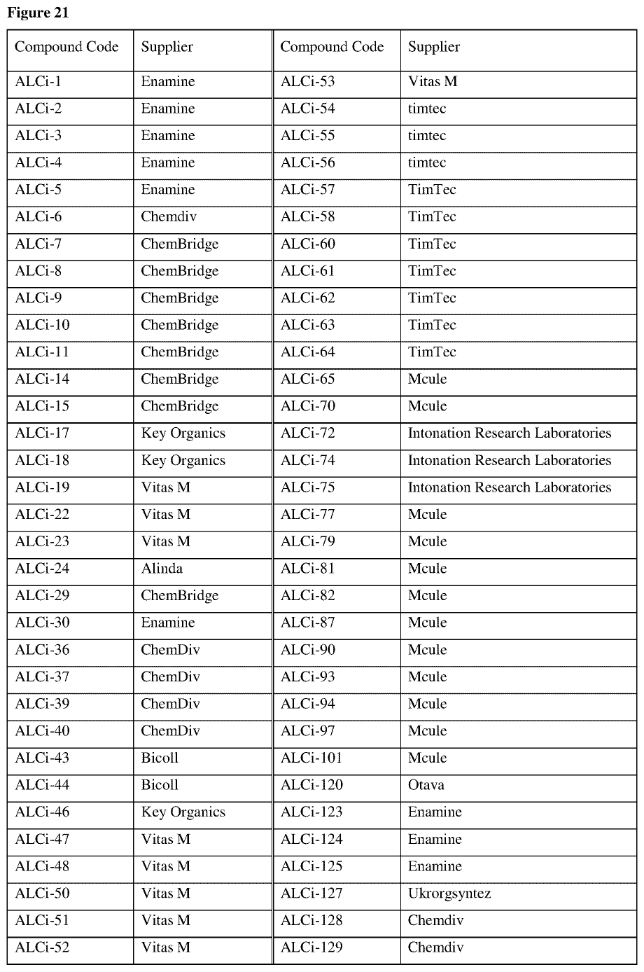

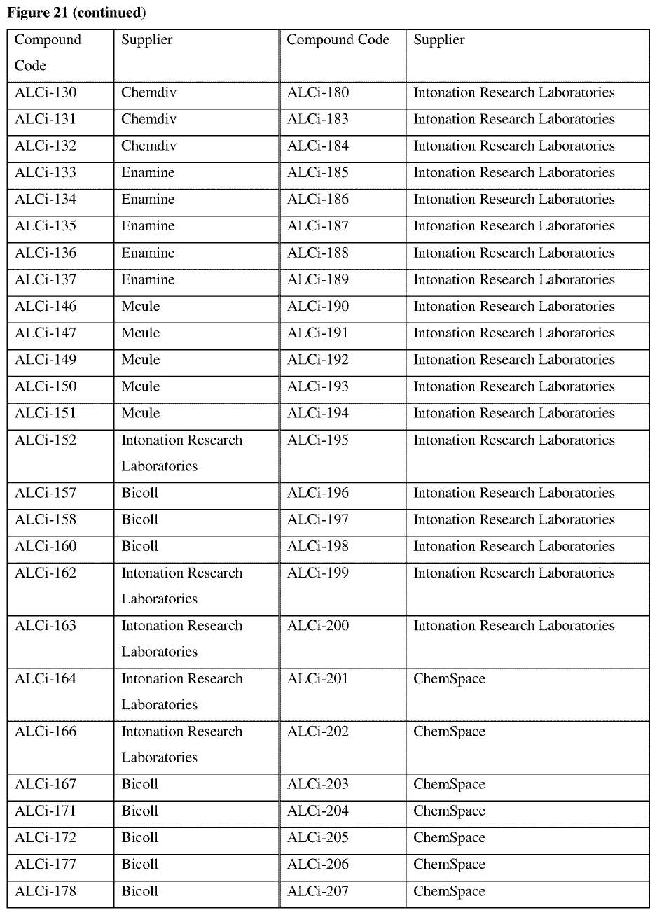

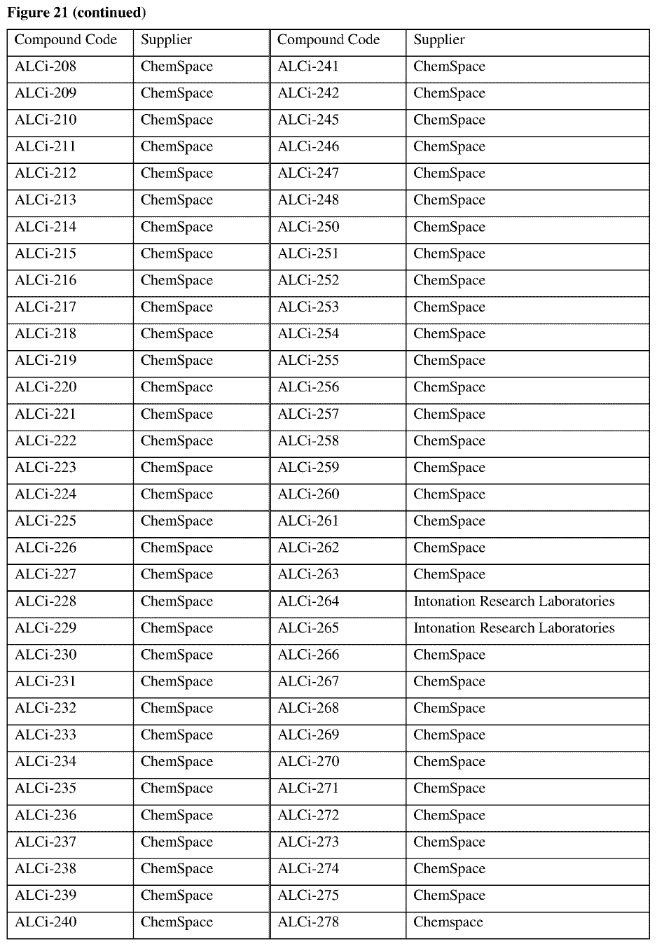

The term “Chromodomain-helicase-DNA-binding protein 1-like” abbreviated CHD1L refers to a protein that is also termed ALC1. The amino acid sequence of human ALC1 is as specified in SEQ ID NO: 1. The 897 amino acid residues long protein consists of an N-terminal Snf2-like DNA dependent ATPase domain spanning amino acid residues 40 to 513, which contains the conserved helicase motifs critical for catalysis (Flaus et al., 2006). This domain is composed of two RecA like lobes ranging from amino acid residues 48 to 261 and 351 to 513, respectively. The structure of a truncated N-terminal lobe of the ATPase domain has been determined by homology modeling in order to identify putative allosteric binding sites, a minimal coordinate file of this model is provided as FIG. 20 to allow the skilled person to identify model compounds within the allosteric binding pocket defined for the first time by the present inventors. The allosteric binding pocket is spatially separated from that part of ALC1 involved in binding ATP. The ATPase domain is followed by a linker region ranging from amino acid residues 514 to 703, which contains a putative coiled-coil region (amino acid residues 638 to 675), and a C-terminal macrodomain (amino acid residues 704 to 897). The macrodomain has been shown to directly interact with the ATPase domain, thereby inhibiting its catalytic function (Lehmann et al., 2017; Singh et al., 2017). This interaction is released upon poly(ADP-ribose) binding to the macrodomain, leading to an activation of the chromatin remodelling enzyme.

The term “alkyl” as used in the context of the present invention refers to a saturated straight or branched carbon chain. Preferably, the chain comprises from 1 to 10 carbon atoms, i.e. 1, 2, 3, 4, 5, 6, 7, 8, 9 or 10, e.g. methyl, ethyl propyl (n-propyl or iso-propyl), butyl(n-butyl, iso-butyl, sec-butyl, tert-butyl), pentyl, hexyl, heptyl, octyl, nonyl, decyl. Alkyl groups are optionally substituted.

The term “heteroalkyl” as used in the context of the present invention refers to a saturated straight or branched carbon chain. Preferably, the chain comprises from 1 to 9 carbon atoms, i.e. 1, 2, 3, 4, 5, 6, 7, 8, or 9, e.g. methyl, ethyl, propyl, iso-propyl, butyl, iso-butyl, sec-butyl, tert-butyl, pentyl, hexyl, heptyl, octyl, nonyl, which is interrupted one or more times, e.g. 1, 2, 3, 4, 5, with the same or different heteroatoms. Preferably, the heteroatoms are selected from O, S, and N, e.g. —(CH₂)ₙ—X—(CH₂)ₘCH₃, with n=0, 1, 2, 3, 4, 5, 6, 7, 8, or 9, m=0, 1, 2, 3, 4, 5, 6, 7, 8, or 9 and X=S, O or NR′ with R′=H or hydrocarbon (e.g. C₁ to C₆ alkyl). In particular “heteroalkyl” refers to —O—CH₃, —OC₂H₅, —CH₂—O—CH₃, —CH₂—O—C₂H₅, —CH₂—O—C₃H₇, —CH₂—O—C₄H₉, —CH₂—O—C₅H₁₁, —C₂H₄—O—CH₃, —C₂H₄—O—C₂H₅, —C₂H₄—O—C₃H₇, —C₂H₄—O—C₄H, etc. Heteroalkyl groups are optionally substituted.

The term “haloalkyl” refers to a saturated straight or branched carbon chain in which one or more hydrogen atoms are replaced by halogen atoms, e.g. by fluorine, chlorine, bromine or iodine. Preferably, the chain comprises from 1 to 10 carbon atoms, i.e. 1, 2, 3, 4, 5, 6, 7, 8, 9 or 10. In particular, “haloalkyl” refers to —CH₂F, —CHF₂, —CF₃, —C₂H₄F, —C₂H₃F₂, —C₂H₂F₃, —C₂HF₄, —C₂F₅, —C₃H₆F, —C₃H₅F₂, —C₃H₄F₃, —C₃H₃F₄, —C₃H₂F₅, —C₃HF₆, —C₃F₇, —CH₂Cl, —CHCl₂, —CCl₃, —C₂H₄Cl, —C₂H₃Cl₂, —C₂H₂Cl₃, —C₂HCl₄, —C₂Cl₅, —C₃H₆Cl, —C₃H₅Cl₂, —C₃H₄Cl₃, —C₃H₃Cl₄, —C₃H₂Cl₅, —C₃HCl₆, and —C₃Cl₇. Haloalkyl groups are optionally substituted.

The term “5, 6, or 7 membered carbocycle” is used in the context of the present invention to refer to “cycloalkyl”, “cycloalkenyl” or “aryl” with 5, 6, or 7 carbon atoms forming a ring.

The term “cycloalkyl” includes cyclopentyl, cyclohexyl, and cycloheptyl. Cycloalkyl groups are optionally substituted.

The term “cycloalkenyl” includes cyclopentenyl, cyclohexenyl, and cycloheptenyl. Cycloalkenyl groups are optionally substituted.

The term “aryl” refers to phenyl. Aryl is optionally substituted, e.g. naphthyl.

The term “5, 6, or 7 membered heterocycle” is used in the context of the present invention to refer to monocyclic “5, 6, or 7 membered heterocycloalkyl” or monocyclic “5, 6, or 7 membered heteroaryl” with 5, 6, or 7 atoms forming a ring.

The term “5, 6, or 7 membered heterocycloalkyl” refers to a saturated monocycle, wherein at least one of the carbon atoms are replaced by 1, or 2 (for the five membered ring) or 1, 2, or 3 (for the six membered ring) or 1, 2, 3, or 4 (for the seven membered ring) of the same or different heteroatoms, preferably selected from O, N and S. Preferred examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, or 2-piperazinyl. Heterocycloalkyl groups are optionally substituted.

The term “heteroaryl” as used in the context of the present invention refers to a 5, 6 or 7-membered aromatic monocyclic ring wherein at least one of the carbon atoms are replaced by 1, 2, or 3 (for the five membered ring) or 1, 2, 3, or 4 (for the six membered ring) of the same or different heteroatoms, preferably selected from O, N and S. Examples of preferred heteroaryls are furanyl, thienyl, oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3,-thiadiazolyl, 1,2,5-thiadiazolyl, pyridinyl, pyrimidinyl, pyrazinyl, 1,2,3-triazinyl, 1,2,4-triazinyl, 1,3,5-triazinyl. Heteroaryls groups are optionally substituted.

If two or more radicals can be selected independently from each other, then the term “independently” means that the radicals may be the same or may be different.

The term “optionally substituted” in each instance if not further specified refers to halogen (in particular F, Cl, Br, or I), —NO₂, —CN, —OR′″, —NR′R″, —COOR′″, CONR′R″, —NR′COR″, —NR″COR′″, —NR′CONR′R″, —NR′SO₂E, —COR′″; —SO₂NR′R″, —OOCR′″, —CR′″R′″OH, —R′″OH, and -E;
R′ and R″ is each independently selected from the group consisting of hydrogen, alkyl, alkenyl, alkynyl, cycloalkyl, heterocycloalkyl, aryl, aralkyl, and heteroaryl or together form a heteroaryl, or heterocycloalkyl;
R′″ and R″″ is each independently selected from the group consisting of hydrogen, alkyl, alkenyl, alkynyl, cycloalkyl, heterocycloalkyl, alkoxy, aryl, aralkyl, heteroaryl, and —NR′R″;
E is selected from the group consisting of alkyl, alkenyl, alkynyl, cycloalkyl, alkoxy, alkoxyalkyl, heterocycloalkyl, an alicyclic system, aryl and heteroaryl; optionally substituted.

“Pharmaceutically acceptable” means approved by a regulatory agency of the Federal or a state government or listed in the U.S. Pharmacopeia (United States Pharmacopeia-33/National Formulary-28 Reissue, published by the United States Pharmacopeia Convention, Inc., Rockville Md., publication date: April 2010) or other generally recognized pharmacopeia for use in animals, and more particularly in humans.

The term “pharmaceutically acceptable salt” refers to a salt of a compound of the present invention. Suitable pharmaceutically acceptable salts of the compound of the present invention include acid addition salts which may, for example, be formed by mixing a solution of a compound described herein or a derivative thereof with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxyethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

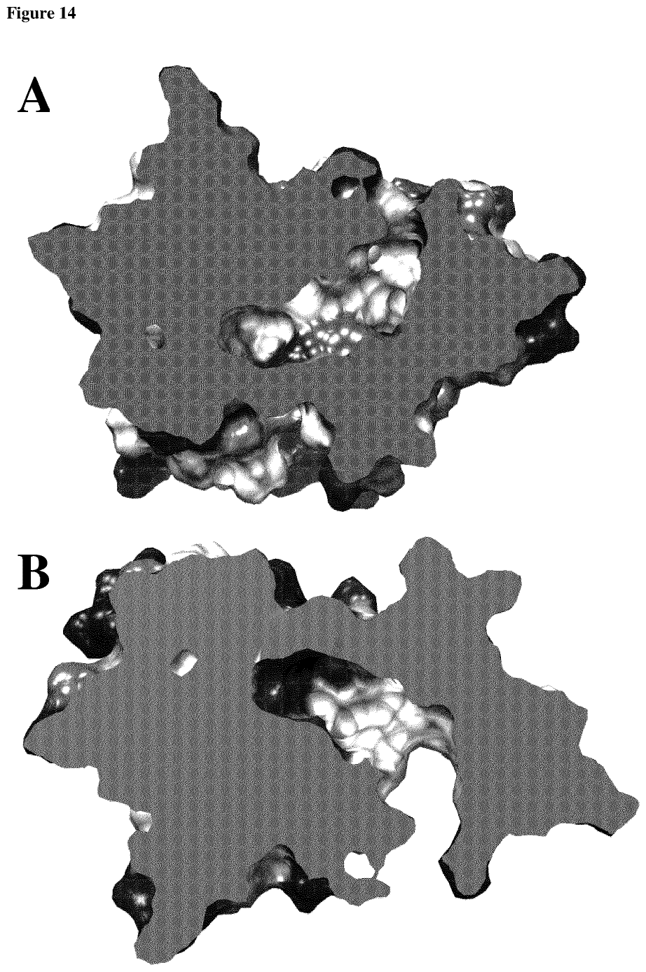

In addition to salt forms, the present invention provides compounds which are in a prodrug form. Prodrugs of the compounds described herein are those compounds that readily undergo chemical changes under physiological conditions to provide a compound of formula (I) to (IV), and especially a compound shown in FIG. 14. A prodrug is an active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters, see Svensson L. A. and Tunek A. (1988) Drug Metabolism Reviews 19(2): 165-194 and Bundgaard H. “Design of Prodrugs”, Elsevier Science Ltd. (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bundgaard H. et al. (1989) J. Med. Chem. 32(12): 2503-2507). Also, drugs containing an acidic NH group, such as imidazole, imide, indole and the like, have been masked with N-acyloxymethyl groups (Bundgaard H. “Design of Prodrugs”, Elsevier Science Ltd. (1985)). Hydroxy groups have been masked as esters and ethers. EP 0 039 051 A2 discloses Mannich-base hydroxamic acid prodrugs, their preparation and use.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, “para position” when referring to the substituent of an aryl means that the substituent occupies the position opposite to the position at which the aryl is linked to the backbone of the compound.

As used herein, a “patient” means any mammal or bird that may benefit from a treatment with the compounds described herein. Preferably, a “patient” is selected from the group consisting of laboratory animals, domestic animals, or primates including chimpanzees and human beings. It is particularly preferred that the “patient” is a human being.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in a subject for a certain amount of time. For example, if a compound described herein is administered to a subject with the aim of preventing a disease or disorder, said disease or disorder is prevented from occurring at least on the day of administration and preferably also on one or more days (e.g. on 1 to 30 days; or on 2 to 28 days; or on 3 to 21 days; or on 4 to 14 days; or on 5 to 10 days) following the day of administration.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

An “effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

### Embodiments of the Invention

The present inventors have identified and characterized within ALC1 a pocket that appears to be involved in allosteric regulation of the ATPase activity of ALC1. Compounds that specifically bind to this pocket are capable of inhibiting the ATPase activity of ALC1. Compounds that bind to the ATPase site of ALC1 and block the ATPase activity have to compete with ATP for binding to the ATPase site. Since the cellular ATP concentration is in the range of 1 to 10 mM depending on the cellular compartment, very high binding affinities in the low nanomolar range are required to successfully prevent ATP from binding to the ATPase site of ALC1. Allosteric inhibitors of ALC1 do not have this limitation since they do not have to prevent ATP from binding but inhibit ALC1's ATPase activity through a different mechanism. by preventing. The present inventors have identified compounds that are capable of specifically binding to the allosteric pocket and determined the spatial and electronic requirements of compounds that fit into this pocket. Thus, by defining the “lock” the inventors were able to define the “keys”, i.e. compounds, fitting into this lock, i.e. the allosteric binding pocket, and that are capable of forming non-covalent bonds or other stabilizing interactions to allow them to specifically bind in the pocket. Using this rational design approach the present inventors identified compounds that were capable of inhibiting ALC1's movement on chromatin and thus led to so-called PARP trapping. These compounds were also tested for their ability to kill two different tumor cell lines one of which was BRCA deficient.

Structure-based computer modeling of ligand-protein interactions is now a core component of modern drug discovery (Charifson and Kuntz, 1997). Computational methods have played a key role in the drug discovery process for a growing number of marketed drugs, including HIV protease inhibitors (Charifson and Kuntz, 1997; Greer et al., 1994; Jorgensen, 2004) and zanamivir (an antiviral neuraminidase inhibitor) (von Itzstein et al., 1993), and in the development of new drug candidates, such as HIV integrase inhibitors (Hazuda et al., 2004; Schames et al., 2004), hepatitis C protease inhibitors (Liverton et al., 2008; Thomson and Perni, 2006), and beta-secretase inhibitors (BACE-1) (Stauffer et al., 2007). There are three major classes of physical computer methods available in the field (listed from fastest to slowest, and least physical to most physical): (1) very fast molecular docking methods, including DOCK, Glide, AutoDock, FlexX, ICN, PMF, and GOLD, Molecular dynamics based free energy methods (MD), such as MM/GBSA or MM/PBSA, in which solvent, protein, and ligand are subject to forces exerted by each other and thermal fluctuations and move in iterative steps as a response to these forces, and (4) absolute binding free energy (ABFE) methods, including alchemical simulations, which are the most expensive computationally, but which include the physics in the most rigorous way that is currently practical. ABFE methods start from an unbound ligand and potentially the unbound structure of the protein to attempt to predict the structures, affinities, and thermal properties of the complexes of interest. These strategies known in the art and in particular the approach described in the experimental section can be used to identify compounds that are allosteric inhibitors of ALC1 by binding to the allosteric binding pocket within ALC1 first identified by the present inventors.

Accordingly, in a first aspect the present invention provides an allosteric inhibitor of ALC1 wherein the inhibitor specifically binds to an allosteric binding pocket formed by an amino acid stretch of human ALC1 spanning amino acid residues 101 to 219 of SEQ ID NO: 1. The term “specifically binds” as used in this context indicates a K_{D} of the compound to full length human ALC1 with an amino acid sequence according to SEQ ID NO: 1 of 200 μM or lower, preferably of 100 μM, more preferably of 50 μM, more preferably of 10 μM or lower, more preferably of 5 μM or lower, even more preferably of 1 μM and even more preferably of 500 nM or lower. The skilled person is well aware of how to measure dissociation constants of small molecules with regard to proteins, which includes surface plasmon resonance. Preferably the K_{D} of a compound of the invention is measured by immobilizing full length human ALC1 on the surface of a chip and the compound is subsequently applied to the immobilized protein. Preferably such measurement is carried out at 37° C.

In a preferred embodiment of the present invention, the allosteric inhibitor of ALC1 exhibits an ID₅₀ value in a FRET based nucleosome remodeling assay of 500 μM or less, preferably 250 μM or less, more preferably 100 μM or less, more preferably 50 μM or less, more preferably 10 UM or less, more preferably 5 μM or less, or even more preferably 1 μM or less.

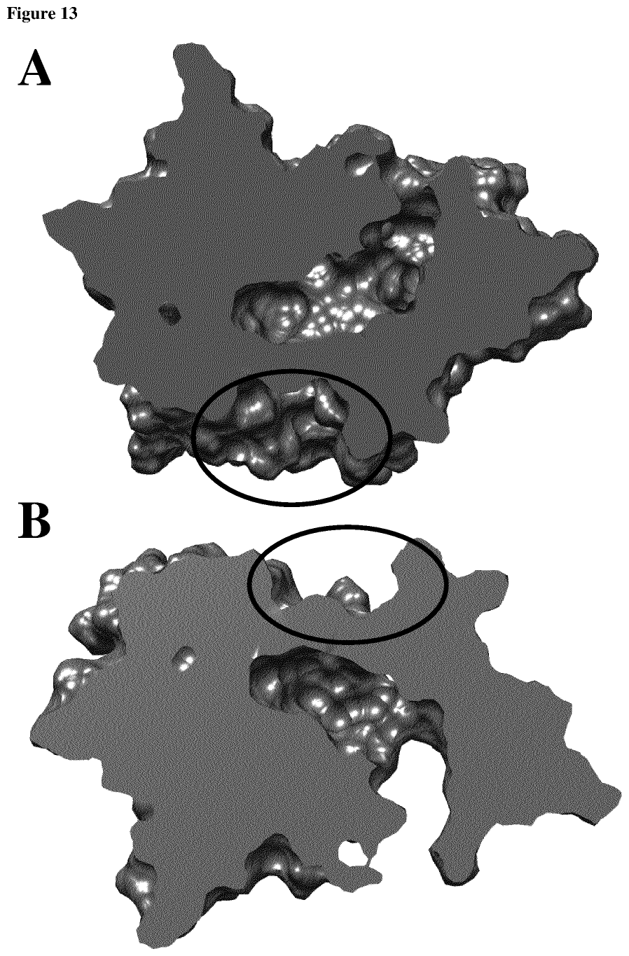

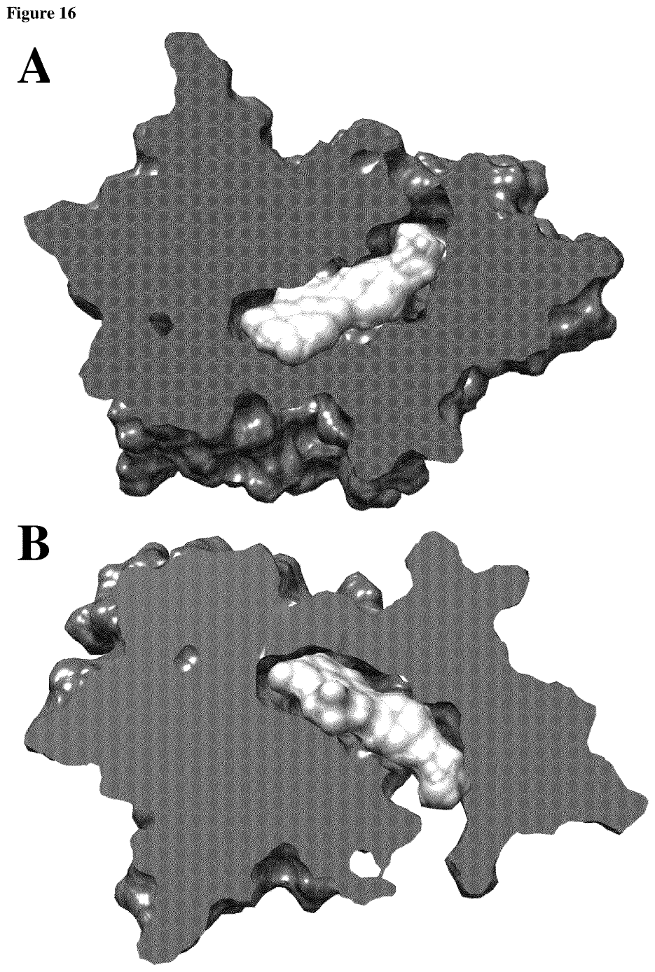

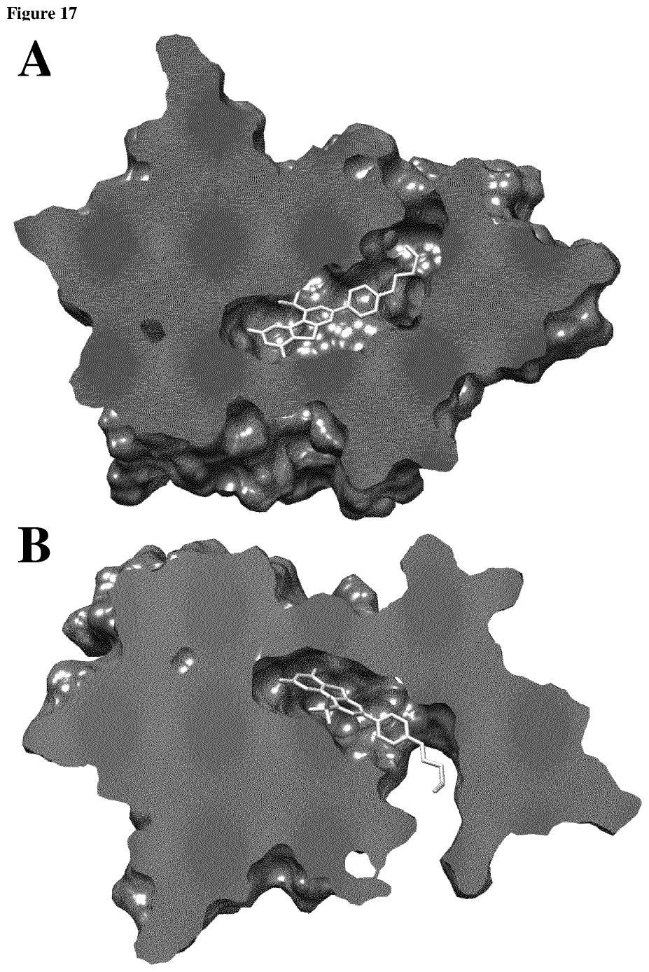

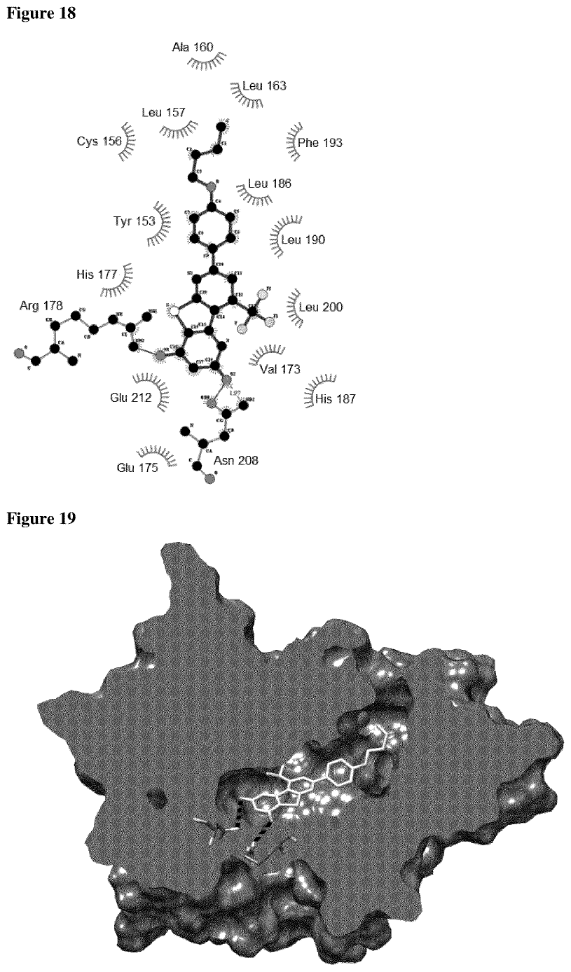

Not every amino acid within the amino acid stretch spanning amino acid residues 101 to 219 of SEQ ID NO: 1 is forming the surface of the allosteric pocket of ALC1 available for binding to the compounds of the invention. This is due to the fact that some amino acids are buried in the pocket and are poorly accessible and others are not even part of the pocket but are located within or on the outside surface of ALC1. Thus, in a preferred embodiment the allosteric binding pocket to which the inhibitors of ALC1 of the present invention specifically bind comprises or consists of amino acids L101, Y153, C156, L157, A160, L163, K164, V173, D174, E175, A176, H177, R178, L179, S183, L186, H187, T189, L190, F193, L200, L201, T202, N208, S209, E212, L213, L216, and F219 of SEQ ID NO: 1, more preferably the binding pocket comprises or consists of Y153, C156, L157, A160, L163, V173, E175. R178, L186, H187, L190 F 193, L200, and E212 of SEQ ID NO: 1. To date, there is no 3D structure of ALC1 available, however the structures of several homologous remodellers have been deposited at the Protein Data Bank (PDB). Thus, the skilled person can model the allosteric binding pocket of ALC1 based on the homology to other chromatin remodelling enzymes. Such models are also depicted in FIG. 13 to FIG. 19 with and without compounds of the invention in the pocket. In particular the volume model of an exemplary compound of the invention shown in FIGS. 16A and B depicts how the skilled person can visualize the suitability of a given compound to fit into the allosteric binding pocket. FIG. 18 only depicts the amino acids available in the pocket for interaction with the compound of the invention and provides further guidance on the selection of compounds that fulfill the steric, hydrophobicity, and hydrophilicity as well as charge requirements in the pocket. A minimal set of structural coordinates of the amino acids of ALC1 involved in binding to the compounds of the invention is provided in FIG. 20.

Based on their orientation within the pocket the different amino acids that are accessible at the surface of the pocket can form different non-covalent bond, in particular hydrogen bonds, ionic interactions, van der Waals interactions and hydrophobic interactions. Accordingly, in a preferred embodiment the inhibitor forms non-covalent bond(s) with one or more amino acids of the allosteric binding pocket, preferably with one or more of the backbone of amino acids L157, A160, K164, V173, D174, H177, R178, L179, L186, N208, and/or E212 of ALC1 and/or the sidechains of L101, Y153, C156, L157, A160, L163, E175, R178, L179, L186, H187, L190, F193, T202, N208, E212, or L213 of ALC1, more preferably with the backbone of D174. H177, and R178 of ALC1, and the sidechain of Y153, E175. R178, H187, T202, N208 and/or E212 of ALC1.

In a preferred embodiment the inhibitor of ALC1 non-covalently binds to:
(i) the aromatic ring of the sidechain of amino acid Y153 of ALC1 ring face-to-face or edge-to-face pi-pi interaction with aromatic carbo- or heterocyclic substituents or forming cation-pi, polar-pi, or halogen-pi interactions with polar, charged, or carbo-halogen substituents;
(ii) the terminal oxygen of the sidechain of amino acid Y153 of ALC1 with a hydrogen bond donating group;
(iii) the carbonyl oxygen of the backbone of H177 with carbo halogens or hydrogen bond donating groups;
(iv) the carbonyl oxygen of the backbone of D174 with carbo halogens or hydrogen bond donating groups;
(v) the sidechain of E175 with a hydrogen bond donating or accepting group;
(vi) the sidechain of R178 with a hydrogen bond donating or accepting group;
(vii) the backbone carbonyl oxygen of R178 with carbo halogens or hydrogen bond donating groups;
(viii) the sidechain of H187 ring face-to-face or edge-to-face pi-pi interaction with aromatic carbo- or heterocyclic substituents or forming cation-pi, polar-pi, or halogen-pi interactions with polar, charged, or carbo-halogen substituents or with a hydrogen bond donating or accepting group
(ix) the sidechain of T202 with a hydrogen bond donating or accepting group;
(x) the sidechain of N208 with a hydrogen bond donating or accepting group; and
(xi) the sidechain of E212 with a hydrogen bond donating or accepting group.

In a preferred embodiment of the first aspect of the invention the allosteric inhibitor has the structure of formula (I):

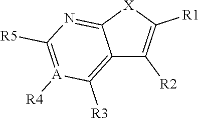

wherein
(i) R₅ comprises an aromatic ring that pi-stacks with the aromatic ring of amino acid Y153; and/or
(ii) N is a hydrogen bond accepting group for the terminal OH of amino acid Y153; and/or
(iii) R₁ comprises a group that is a hydrogen bond donating group to the backbone carbonyl oxygen of amino acid H177 of ALC1; and/or
(iv) R₃ comprises a carbohalogen or hydrogen bond donating group that binds to the carbonyl oxygen of the backbone of amino acid D174 of ALC1; and/or
(v) R₁ and R₂ together form an aromatic or heteroaromatic monocyle comprising a hydrogen bond donating or accepting group especially at or adjacent to the R₁ position which can act as a hydrogen bond donating or accepting group to the side chain of amino acid E175 of ALC1 and/or amino acid R178 of ALC1; and/or
(vi) R₁ and R₂ together form a substituted carbo- or heteromonocycle comprising a hydrogen bond donating or accepting group, preferably at or adjacent to the R₁ position which is a hydrogen bond donating or accepting group to the side chain of amino acid R178 of ALC1; and/or
(vii) R₃ comprises an aromatic ring that pi-stacks with the aromatic ring amino acid H187 of ALC1 or is an electron poor substituent, especially at, forming polar-pi or cation-pi interactions with the aromatic ring of amino acid H187 of ALC1;
(viii) R₁ and R₂ together form an aromatic or heteroaromatic monocycle comprising a hydrogen bond donating or accepting group especially at or adjacent to the R₂ position which can act as a hydrogen bond donating or accepting group to the side chain of amino acid T202 and/or amino acid N208 of ALC1;
(ix) R₄ is H or —C₁₋₃-alkyl, preferably H; and/or
(x) R₅ is —(CH₂)ₘ-L, or —(CH₂)ₘ—(CH═CH)-L, wherein m is 0, 1 or 2, preferably 0 or 1, and L is a 5, 6 or 7 membered carbo- or heterocycle, or adamantyl, optionally substituted, preferably with 1, 2, 3 or 4 substituents independently selected from the group consisting of —OH, —NO₂, —CN, —CO—OR₆, —Br, —Cl, —F, —I, —R₉, —O—R₉, and ═O, or two adjacent substituents form a 5, 6 or 7 membered carbo- or heterocycle; and/or
(xi) R₄ and R₅ together form a 5, 6, or 7 membered carbocycle, optionally substituted, preferably with 1, 2, or 3 substituents independently selected from the group consisting of —OH, —NO₂, —CN, —Br, —Cl, —F, —I, —O—R₉, —R₉, ═CH—R_{A}, preferably R₄ and R₅ together form a C₅₋₇-cycloalkyl; and/or
(xii) X is S or N;
(xiii) A is N or C.

In a further aspect or in a preferred embodiment of the first aspect of the invention, the invention is directed to the allosteric inhibitor having the structure of formula (I):

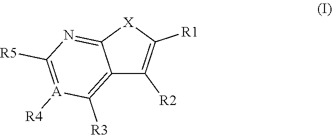

and isomers, salts, solvates, chemically protected forms, and prodrugs thereof, wherein:
X is N or S;
A is C or N;
R₁ is —CO—OR₆, —CO—R₇, or —CO—NR₆R_{A}, preferably R₁ is —CO—OR₆;
R₂ is —R₇, —NHR₈, —O—R₇, —C—O—R₇, Br, —C₃₋₈-cycloalkyl (preferably cyclopropyl), or —C₄₋₈-cycloalkenyl (preferably cyclohexenyl);
or
R₁ and R₂ together form a 5, 6 or 7 membered carbo- or heterocycle, optionally substituted, preferably with 1, 2 or 3 substituents independently selected from the group consisting of —OH, —NO₂, —CN, —Br, —Cl, —F, —I, —O—C₁₋₃-alkyl, ═O, [—O—CH₂—CH₂]—NH—CH(OH)—O-tBu;
R₃ is H, ═O, —OH, —O—R₇, —R₇, or —(CH₂)ₘ-L, wherein m is 0, 1 or 2, and L is a 5, 6 or 7 membered carbo- or heterocycle, optionally substituted, preferably with 1, 2, or 3 substituents independently selected from the group consisting of —OH, —NO₂, —CN, —Br, —Cl, —F, —I, —O—C₁₋₃-alkyl, -hydroxyC₁₋₃-alkyl and ═O;
R₄ is H or —C₁₋₃-alkyl, preferably H;
R₅ is —(CH₂)ₘ-L, or —(CH₂)ₘ—(CH═CH)-L, wherein m is 0, 1 or 2, preferably 0 or 1, and L is a 5, 6 or 7 membered carbo- or heterocycle, adamantyl, C₁₋₄-alkyl, or —N(CH₃)₂, optionally substituted, preferably with 1, 2, 3 or 4 substituents independently selected from the group consisting of —OH, —NO₂, —CN, —CO—OR₆, —Br, —Cl, —F, —I, —R₉, —O—R₉, ═O, and [—O—CH₂—CH₂]_{q}—NH-biotin with q being 1, 2, 3, or 4, or two adjacent substituents form a 5, 6 or 7 membered carbo- or heterocycle;
or
R₄ and R₅ together form a 5, 6, or 7 membered carbocycle, optionally substituted, preferably with 1, 2, or 3 substituents independently selected from the group consisting of —OH, —NO₂, —CN, —Br, —Cl, —F, —I, —O—R₉, —R₉, ═CH—R_{A}, and —CH₂—R_{A}, preferably R₄ and R₅ together form a C₅₋₇-cycloalkyl;
R₆ is H, —C₁₋₆-alkyl, —C₂₋₆-alkenyl, —C₂₋₆-alkynyl, optionally substituted, preferably R₆ is H;
R₇ is —C₁₋₃-alkyl, —C₂₋₃-alkenyl, —C₂₋₃-alkynyl, optionally substituted, preferably with 1, 2, or 3 substituents independently selected from the group consisting of —Br, —Cl, —F, —I, —CH₃, —OCH₃, or —SCH₃;
R₈ is H or C₁₋₆-alkyl, preferably H,
R₉ is —C₁₋₆-alkyl, —C₂₋₆-alkenyl, —C₂₋₆-alkynyl, —C₁₋₆-alkyl-aryl, or C₁₋₆-alkyl-heteroaryl (preferably isooxazole, thiazole, tetrazole, 1,2,4-thiadiazole, 1,2,3-thiadiazole, 1,2,5-thiadiazole, pyridine, 1,2,4-oxadiazole, pyrazine, or pyrazole), optionally substituted with 1, 2 or 3 substituents selected from the group consisting of —Br, —Cl, —F, —I, —NO₂, —CN, —CONH₂, —CONH—C₁₋₃-alkyl (preferably-CONH—CH₃), —NH—CO—C₁₋₃-alkyl (preferably-NH—CO—CH₃), —C₁₋₆-alkyl (preferably-CH₃, ethyl, propyl, t-butyl, or pentyl), —C₁₋₃-haloalkyl (preferably-CF₃, or —CHF₂), —O—CHF₂, —O—CF₃, carbocycle (preferably cyclopropyl, cyclohexyl or phenyl), —O-carbocycle (preferably phenoxy), heterocycle (preferably pyrazolyl), —CO-heterocycle (preferably-CO-(1-pyrrolidinyl)), —SO₂—CH₃, —SO₂—N(CH₃)₂, —O—C₁₋₄-alkyl (preferably —OCH₃), —O—C₁₋₃-alkyl-O—C₁₋₃-alkyl (preferably —O—CH₂—O—CH₃), —SCH₃, or when R₉ is —C₁₋₆-alkyl-aryl, then two adjacent substituents on the aryl moiety can form a 5, 6 or 7 membered carbo- or heterocycle, which is optionally substituted;
R_{A} is H, carbo- or heterocycle, optionally substituted, preferably with 1, 2 or 3 substituents independently selected from the group consisting of —OH, —NO₂, —CN, —Br, —Cl, —F, —I, —R₉, —O—R₇, —O—(CH₂) %—R₉, —SO₂NH₂, and ═O, wherein o is 0 or 1.

In yet another aspect or in a more preferred embodiment of the first aspect of the invention, the invention is directed to an allosteric inhibitor having the structure of formula (I):

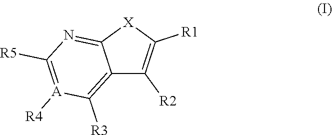

and isomers, salts, solvates, chemically protected forms, and prodrugs thereof, wherein:
X is N or S;
A is C or N;
R₁ is —CO—OR₆, —CO—R₇, or —CO—NR₆R_{A}, preferably R₁ is —CO—OR₆;
R₂ is —R₇, —NHR₈, —O—R₇, —C—O—R₇;
or
R₁ and R₂ together form a 5, 6 or 7 membered carbo- or heterocycle, optionally substituted, preferably with 1, 2 or 3 substituents independently selected from the group consisting of —OH, —NO₂, —CN, —Br, —Cl, —F, —I, —O—C₁₋₃-alkyl, and ═O;
R₃ is H, ═O, —OH, —O—R₇, —R₇, or —(CH₂)ₘ-L, wherein m is 0, 1 or 2, and L is a 5, 6 or 7 membered carbo- or heterocycle, optionally substituted, preferably with 1, 2, or 3 substituents independently selected from the group consisting of —OH, —NO₂, —CN, —Br, —Cl, —F, —I, —O—C₁₋₃-alkyl, -hydroxyC₁₋₃-alkyl and ═O:
R₄ is H or —C₁₋₃-alkyl, preferably H;
R₅ is —(CH₂)ₘ-L, or —(CH₂)ₘ—(CH═CH)-L, wherein m is 0, 1 or 2, preferably 0 or 1, and L is a 5, 6 or 7 membered carbo- or heterocycle, or adamantyl, optionally substituted, preferably with 1, 2, 3 or 4 substituents independently selected from the group consisting of —OH, —NO₂, —CN, —CO—OR₆, —Br, —Cl, —F, —I, —R₉, —O—R₉, and ═O, or two adjacent substituents form a 5, 6 or 7 membered carbo- or heterocycle;
or
R₄ and R₅ together form a 5, 6, or 7 membered carbocycle, optionally substituted, preferably with 1, 2, or 3 substituents independently selected from the group consisting of —OH, —NO₂, —CN, —Br, —Cl, —F, —I, —O—R₉, —R₉, ═CH—R_{A}, preferably R₄ and R₅ together form a C₅₋₇-cycloalkyl;
R₆ is H, —C₁₋₆-alkyl, i.e. C₁-, C₂-, C₃-, C₄-, C₅- or C₆-alkyl, —C₂₋₆-alkenyl, i.e. C₂-, C₃-, C₄-, C₅- or C₆-alkenyl, —C₂₋₆-alkynyl, i.e. C₂-, C₃-, C₄-, C₅- or C₆-alkynyl, optionally substituted, preferably R₆ is H;
R₇ is —C₁₋₃-alkyl, i.e. C₁-, C₂-, C₃-alkyl, —C₂₋₃-alkenyl, i.e. C₂-, C₃-alkenyl, —C₂₋₃-alkynyl, i.e. C₂-, C₃-alkynyl, optionally substituted, preferably with 1, 2, or 3 substituents independently selected from the group consisting of —Br, —Cl, —F, —I, —CH₃, —OCH₃, or —SCH₃;
R₈ is H or C₁₋₆-alkyl, preferably H,
R₉ is —C₁₋₆-alkyl, i.e. C₁-, C₂-, C₃-, C₄-, C₅- or C₆-alkyl, —C₂₋₆-alkenyl, i.e. C₂-, C₃-, C₄-, C₅- or C₆-alkenyl, —C₂₋₆-alkynyl, i.e. C₂-, C₃-, C₄-, C₅- or C₆-alkynyl, or —C₁₋₆-alkyl-aryl, i.e. C₁-, C₂-, C₃-, C₄-, C₅- or C₆-alkyl-aryl, optionally substituted with 1, 2 or 3 substituents selected from the group consisting of —Br, —Cl, —F, —I, —CH₃, —OCH₃, or —SCH₃
R_{A} is H, carbo- or heterocycle, optionally substituted, preferably with 1, 2 or 3 substituents independently selected from the group consisting of —OH, —NO₂, —CN, —Br, —Cl, —F, —I, —R₉, —O—R₇, —O—(CH₂)ₒ—R₉, —SO₂NH₂, and ═O, wherein o is 0 or 1.
   In a preferred embodiment of the first aspect or the further aspect of the invention X is N.
   In a preferred embodiment of the first aspect or the further aspect of the invention A is C.
   In a preferred embodiment of the first aspect of the invention R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H;

In a preferred embodiment of the first aspect or the further aspect of the invention R₁ is —CO—OH.

In a preferred embodiment of the first aspect or the further aspect of the invention X is N and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H.

In a preferred embodiment of the first aspect or the further aspect of the invention A is C and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H.

In a preferred embodiment of the first aspect or the further aspect or the yet another aspect of the invention X is N, A is C and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H.

In a preferred embodiment of the first aspect or the further aspect or the yet another aspect of the invention X is N, A is C and R₁ is —CO—OH or —CO—NH₂.

In a preferred embodiment of the first aspect or the further aspect or the yet another aspect of the invention R₂ is —NHR₈, wherein R₈ is H or C₁₋₆-alkyl, preferably H.

In a preferred embodiment of the first aspect or the further aspect or the yet another aspect of the invention X is N and R₂ is —NHR₈, wherein R₈ is H or C₁₋₆-alkyl, preferably H.

In a preferred embodiment of the first aspect or the further aspect or the yet another aspect of the invention A is C and R₂ is —NHR₈, wherein R₈ is H or C₁₋₆-alkyl, preferably H.

In a preferred embodiment of the first aspect or the further aspect or the yet another aspect of the invention R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H.

In a preferred embodiment of the first aspect or the further aspect or the yet another aspect of the invention R₁ is —CO—OH and R₂ is —NHR₈, wherein R₈ is H or C₁₋₆-alkyl, preferably H.

In a preferred embodiment of the first aspect or the further aspect or the yet another aspect of the invention X is N and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H.

In a preferred embodiment of the first aspect or the further aspect or the yet another aspect of the invention A is C and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H.

In a preferred embodiment of the first aspect or the further aspect or the yet another aspect of the invention X is N, A is C and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H.

In a preferred embodiment of the first aspect or the further aspect or the yet another aspect of the invention X is N, A is C and R₁ is —CO—OH or —CO—NH₂ and R₂ is —NHR₈, wherein R₈ is H or C₁₋₆-alkyl, preferably H.

In a preferred embodiment of the first aspect or the further aspect or the yet another aspect of the invention R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and —O, preferably —OH and ═O.

In a preferred embodiment of the first aspect or the further aspect or the yet another aspect of the invention X is N, and R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and —O, preferably —OH and ═O.

In a preferred embodiment of the first aspect or the further aspect or the yet another aspect of the invention A is C, and R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and —O, preferably —OH and ═O.

In a preferred embodiment of the first aspect or the further aspect or the yet another aspect of the invention X is N, A is C and R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O.

In a preferred embodiment of the first aspect or the further aspect or the yet another aspect of the invention R₁ and R₂ together form an uracil or 3-deazauracil.

In a preferred embodiment of the first aspect or the further aspect or the yet another aspect of the invention X is N, R₁ and R₂ together form an uracil or 3-deazauracil.

In a preferred embodiment of the first aspect or the further aspect or the yet another aspect of the invention A is C, and R₁ and R₂ together form an uracil or 3-deazauracil.

In a preferred embodiment of the first aspect or the further aspect or the yet another aspect of the invention X is N, A is C and R₁ and R₂ together form an uracil or 3-deazauracil.

In a preferred embodiment of the first aspect or the further aspect or the yet another aspect of the invention R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ is —CO—OH and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ is —CO—OH or —CO—NH₂ and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents selected from the independently group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ is —CO—OH and R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, —O, —OH, —R₇, or —(CH₂)ₘ-L, wherein m is 0 and L is phenyl or a 5, 6 or 7 membered heteroaryl, preferably phenyl, optionally substituted, preferably from with 1, 2, or 3 substituents independently selected the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, —O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, —O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ is —CO—OH or —CO—NH₂ and R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ and R₂ together form an uracil or 3-deazauracil and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₁ and R₂ together form an uracil or 3-deazauracil and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C R₁ and R₂ together form an uracil or 3-deazauracil and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ and R₂ together form an uracil or 3-deazauracil and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ is —CO—OH and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ is —CO—OH or —CO—NH₂R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H, R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ is —CO—OH, R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ is —CO—OH or —CO—NH₂ and R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₁, R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and —O, preferably —OH and —O and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ and R₂ together form an uracil or 3-deazauracil and R₃ is —C₁₋₃-alkyl, i.e. C₁-, C₂-, C₃-alkyl, or (CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, and R₁ and R₂ together form an uracil or 3-deazauracil and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is C, R₁ and R₂ together form an uracil or 3-deazauracil and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is C, R₁ and R₂ together form an uracil or 3-deazauracil and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect of the invention R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention R₁ is —CO—OH and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is C, R₁ is —CO—OH or —CO—NH₂ and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is C, R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention R₁ is —CO—OH, R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is C, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, —O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is C, R₁ is —CO—OH or —CO—NH₂ and R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃. In a preferred embodiment of the first aspect or the further aspect or the yet another aspect of the invention R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is C, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is C, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention R₁ and R₂ together form an uracil or 3-deazauracil and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, R₁ and R₂ together form an uracil or 3-deazauracil and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is C, R₁ and R₂ together form an uracil or 3-deazauracil and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is C and R₁ and R₂ together form an uracil or 3-deazauracil and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is N and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H.

In a preferred embodiment of the first aspect or the further aspect or the yet another aspect of the invention X is N, A is N and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H.

In a preferred embodiment of the first aspect or the further aspect or the yet another aspect of the invention X is N, A is N and R₁ is —CO—OH or —CO—NH₂.

In a preferred embodiment of the first aspect or the further aspect or the yet another aspect of the invention A is N and R₂ is —NHR₈, wherein R₈ is H or C₁₋₆-alkyl, preferably H.

In a preferred embodiment of the first aspect or the further aspect or the yet another aspect of the invention A is N and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H.

In a preferred embodiment of the first aspect or the further aspect or the yet another aspect of the invention X is N, A is N and R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H.

In a preferred embodiment of the first aspect or the further aspect or the yet another aspect of the invention X is N, A is N and R₁ is —CO—OH or —CO—NH₂ and R₂ is —NHR₈, wherein R₈ is H or C₁₋₆-alkyl, preferably H.

In a preferred embodiment of the first aspect or the further aspect or the yet another aspect of the invention A is N, and R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O.

In a preferred embodiment of the first aspect or the further aspect or the yet another aspect of the invention X is N, A is N and R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and —O, preferably —OH and ═O.

In a preferred embodiment of the first aspect or the further aspect or the yet another aspect of the invention A is N, and R₁ and R₂ together form an uracil or 3-deazauracil.

In a preferred embodiment of the first aspect or the further aspect or the yet another aspect of the invention X is N, A is N and R₁ and R₂ together form an uracil or 3-deazauracil.

In a preferred embodiment of the first aspect or the further aspect or the yet another aspect of the invention A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ is —CO—OH or —CO—NH₂ and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from group consisting the of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ is —CO—OH or —CO—NH₂ and R₂ is —NHR₈, wherein R₈ is H or C₁₋₆-alkyl, preferably H and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and —O, preferably —OH and ═O and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N R₁ and R₂ together form an uracil or 3-deazauracil and R₃ is H, ═O, —OH, —R₇, or —(CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ and R₂ together form an uracil or 3-deazauracil and R₃ is H, ═O, —OH, —R₇, or (CH₂)ₘ-L, wherein m is 0 and L is phenyl or a 5, 6 or 7 membered heteroaryl, preferably phenyl, optionally substituted, preferably with 1, 2, or 3 substituents independently selected from the group consisting of —OH, —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ is —CO—OH or —CO—NH₂ R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H, R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention R₁ is —CO—OH, R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ is —CO—OH or —CO—NH₂ and R₂ is —NHR₈, wherein R₈ is H or C₁₋₆-alkyl, preferably H and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and —O, preferably —OH and —O and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₁ and R₂ together form an uracil or 3-deazauracil and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention X is N, A is N, R₁ and R₂ together form an uracil or 3-deazauracil and R₃ is —C₁₋₃-alkyl, i.e. C₁-, C₂-, C₃-alkyl, or —(CH₂)ₘ-L, wherein m is 0 and L is phenyl or a 5 membered heteroaryl, preferably phenyl, optionally substituted, preferably with 1, 2, or 3 substituents independently selected from the group consisting of —NO₂, —CN, —Br, —Cl, —F, —I, —O—C₁₋₃-alkyl, and -hydroxyC₁₋₃-alkyl.

In a preferred embodiment of the first aspect or the further aspect or the yet another aspect of the invention A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is N, R₁ is —CO—OH or —CO—NH₂ and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is N, R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is N, R₁ is —CO—OR₆, or —CO—NR₆R_{A} preferably R₆ is H, —C₁₋₃-alkyl, i.e. C₁-, C₂-, C₃-, -alkyl, —C₂₋₃-alkenyl, i.e. C₂-, C₃-, -alkenyl, —C₂₋₃-alkynyl, i.e. C₂-, C₃-optionally substituted, preferably R₆ is H and R₂ is —NHR₈, wherein R₈ is H or C₁₋₆-alkyl, preferably H and R₃ is H, —O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is N, R₁ is —CO—OH or —CO—NH₂ and R₂ is —NHR₈, wherein R₈ is H or C₁₋₆-alkyl, preferably H and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is N, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is N, R₁ and R₂ together form a 6 membered aryl or heteroaryl moiety, optionally substituted, preferably with 1, 2 or 3 substituents independently selected from the group consisting of —OH, —NO₂, —CN, —Br, —Cl, —F, —I, —O—C₁₋₃-alkyl, and ═O, preferably —OH and ═O and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention A is N, R₁ and R₂ together form an uracil or 3-deazauracil and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention X is N, A is N and R₁ and R₂ together form an uracil or 3-deazauracil and R₃ is H, ═O, —OH, thiophenyl, phenyl, 3,4,5 hydroxymethyl phenyl, CF₂H, or CF₃.

In a preferred embodiment of the first aspect or the further aspect or the yet another aspect of the invention R₅ is —(CH₂)ₘ-L, wherein m is 0, or 1 or —(CH₂)—(CH═CH)-L and L is phenyl or a 5, or 6 membered heteroaryl, or adamantyl, optionally substituted, preferably with 1, 2, or 3 substituents independently selected from the group consisting of —OH, —NO₂, —CN, —CO—OR₆, —Br, —Cl, —F, —I, —R₉, —O—R₉, and ═O, or two adjacent substituents form a 5, 6 or 7 membered carbo- or heterocycle.

In a preferred embodiment of the first aspect or the further aspect or the yet another aspect of the invention R₄ and R₅ together form a 5 or 6 membered non-substituted or mono substituted heterocycloalkyl, preferably with ═CH—R_{A} substituted at the R₄ position.

In a preferred embodiment of the first aspect or the further aspect or the yet another aspect of the invention R_{A} is a substituted carbocycle with 1 or 2 substituents independently selected from the group consisting of —Br, —Cl, —F, —O—(CH₂)ₒ—R₉, or —SCH₃, wherein o is 0 or 1.

In a preferred embodiment of the first aspect or the further aspect or the yet another aspect of the invention R₄ and R₅ together form a 5 or 6 membered non-substituted or mono substituted heterocycloalkyl, preferably with ═CH—R_{A} substituted at the R₄ position, wherein R_{A} is a substituted carbocycle with 1 or 2 substituents independently selected from the group consisting of —Br, —Cl, —F, —O—(CH₂) %—R₉, or —SCH₃, wherein o is 0 or 1.

In a preferred embodiment of the first aspect or the further aspect or the yet another aspect of the invention R₉ is —C₁₋₄-alkyl, i.e. C₁-, C₂-, C₃-, or C₄-alkyl, —C₂₋₄-alkenyl, i.e. C₂-, C₃-, or C₄-alkenyl, or —C₁₋₆-alkyl-aryl, i.e. C₁-, C₂-, C₃-, C₄-, C₅- or C₆-alkyl-aryl, optionally substituted with 1 or 2 substituents selected from the group consisting of —Cl, —CH₃, —OCH₃, or —SCH₃.

In a preferred embodiment of the first aspect or the further aspect or the yet another aspect of the invention R₅ is —(CH₂)ₘ-L, wherein m is 0, or 1 or —(CH₂)—(CH═CH)-L and L is phenyl or a 5, or 6 membered heteroaryl, or adamantyl, optionally substituted, preferably with 1, 2, or 3 substituents independently selected from the group consisting of —OH, —NO₂, —CN, —CO—OR₆, —Br, —Cl, —F, —I, —R₉, —O—R₉, and —O, or two adjacent substituents form a 5, 6 or 7 membered carbo- or heterocycle, wherein R₉ is —C₁₋₄-alkyl, i.e. C₁-, C₂-, C₃-, or C₄-alkyl, —C₂₋₄-alkenyl, i.e. C₂-, C₃-, or C₄-alkenyl, or —C₁₋₆-alkyl-aryl, i.e. C₁-, C₂, C₃-, C₄-, C₅- or C₆-alkyl-aryl, optionally substituted with 1 or 2 substituents selected from the group consisting of —Cl, —CH₃, —OCH₃, or —SCH₃.

In a particularly preferred embodiment of the first aspect of the invention the allosteric inhibitor has the structure of formula (Ia):

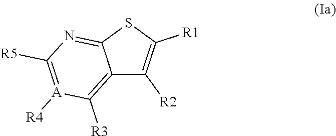

and isomers, salts, solvates, chemically protected forms, and prodrugs thereof, wherein:
A is C or N;
R₁ is —COOH
R₂ is —CH₃ or —NH₂
or
R₁ and R₂ together form a uracil or 3-deazauracil;
R₃ is H, ═O, —OH, thiophenyl, phenyl, 3,4,5 hydroxymethyl phenyl, CF₂H, or CF₃;
R₄ is H
R₅ is -L, or —CH₂—(CH═CH)-L, wherein L is a 6 membered carbo- or heterocycle, or adamantyl, optionally substituted with 1 or 2 substituents independently selected from the group consisting of —OH, —NO₂, —Br, —Cl, —F, CH₃, —O—R₉, and ═O, or two adjacent substituents form a 5 or 6 membered heterocycle;
or
R₄ and R₅ together form a 5 or 6 membered non-substituted or mono substituted heterocycloalkyl, preferably with ═CH—R_{A} substituted at the R₄ position;
R₉ is —C₁₋₄-alkyl, i.e. C₁-, C₂-, C₃-, or C₄-alkyl, —C₂₋₄-alkenyl, i.e. C₂-, C₃-, or C₄-alkenyl, or —C₁₋₆-alkyl-aryl, i.e. C₁-, C₂-, C₃-, C₄-, C₅- or C₆-alkyl-aryl, optionally substituted with 1 or 2 substituents selected from the group consisting of —Cl, —CH₃, —OCH₃, or —SCH₃;
R_{A} is a substituted carbocycle with 1 or 2 substituents independently selected from the group consisting of —Br, —Cl, —F, —O— (CH₂)ₒ—R₉, or —SCH₃, wherein o is 0 or 1.

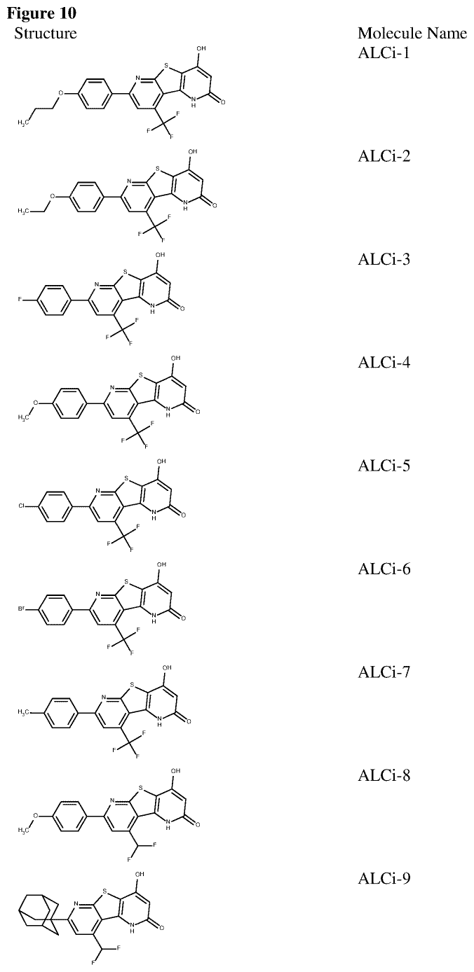

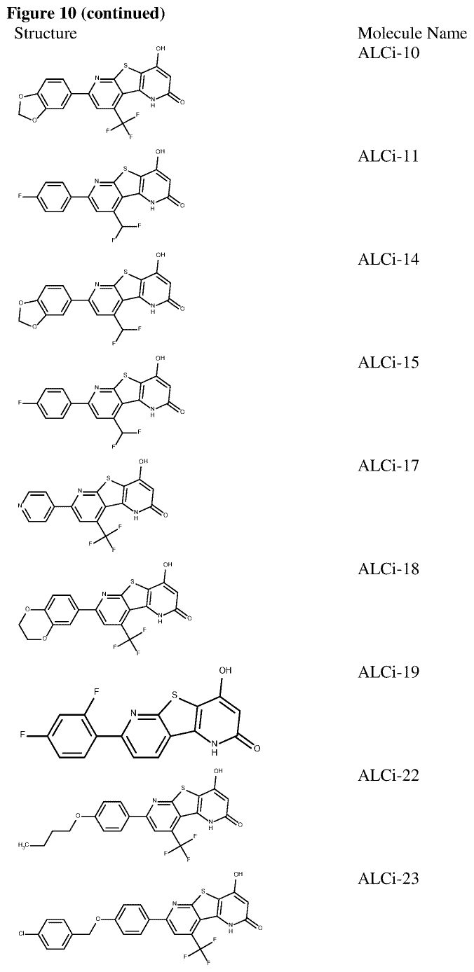

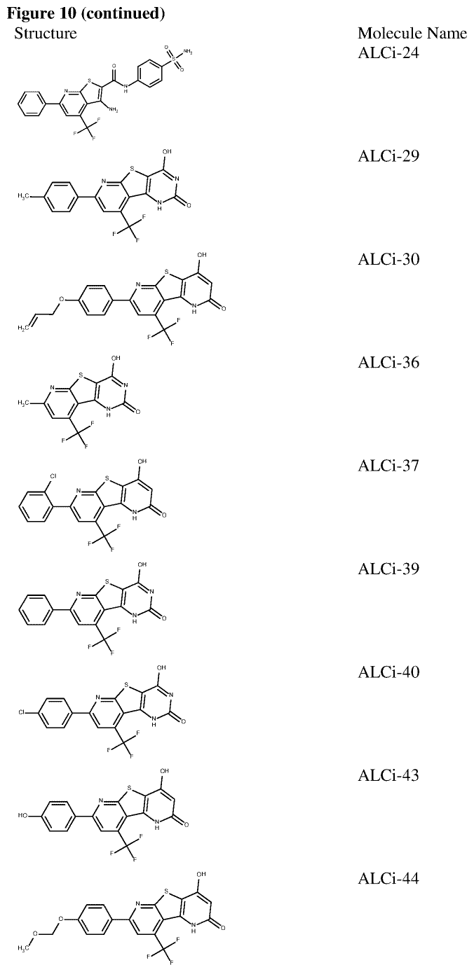

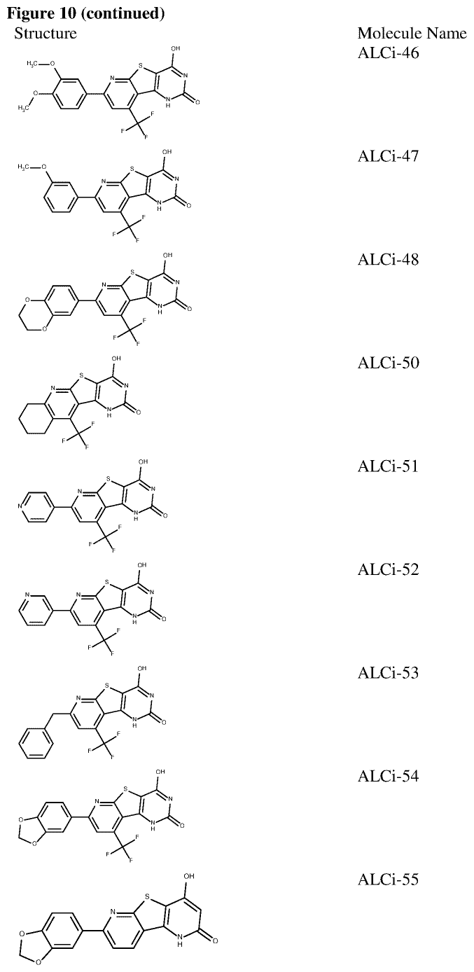

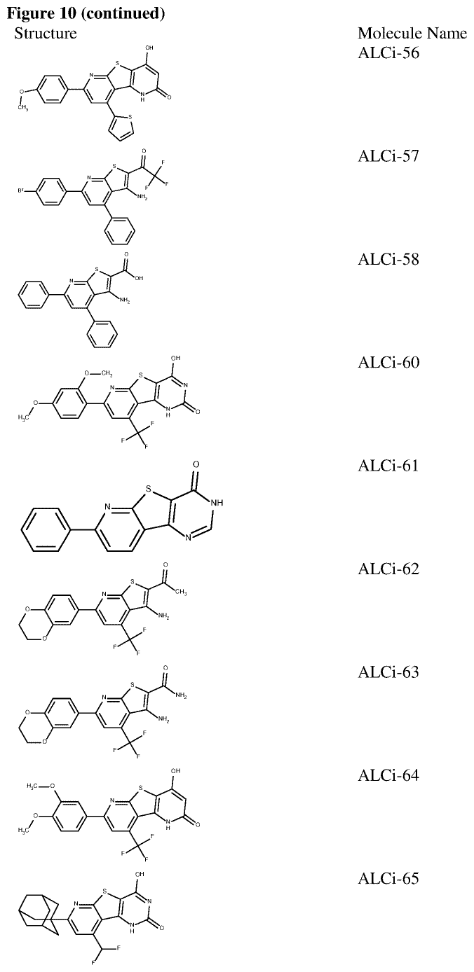

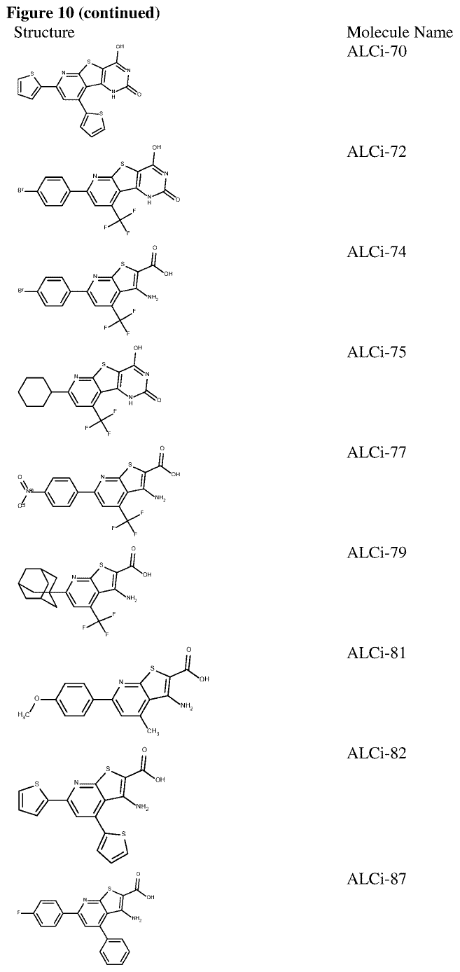

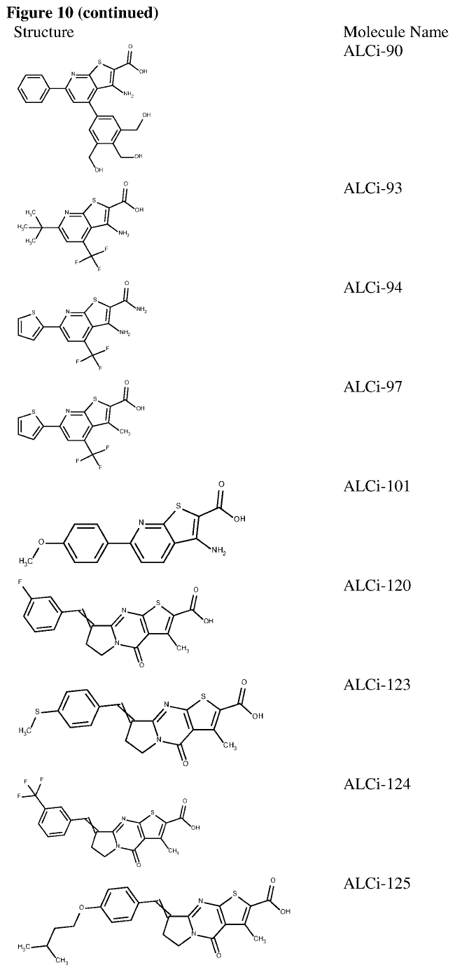

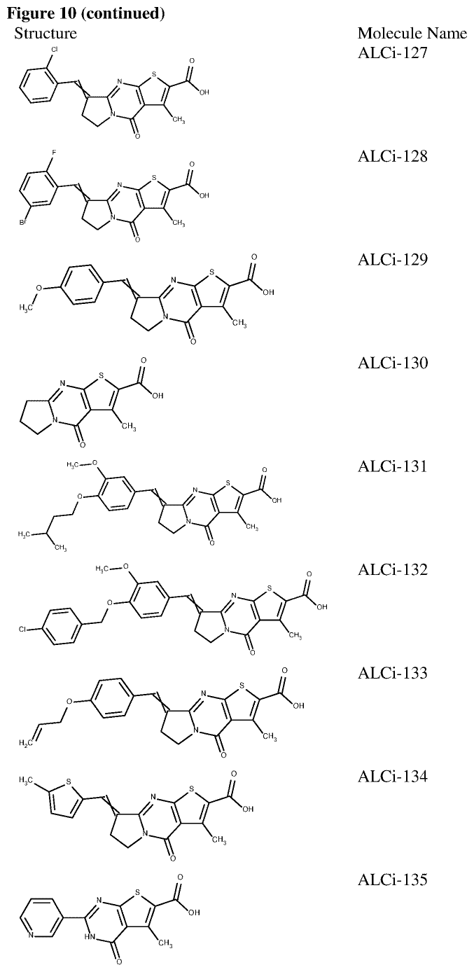

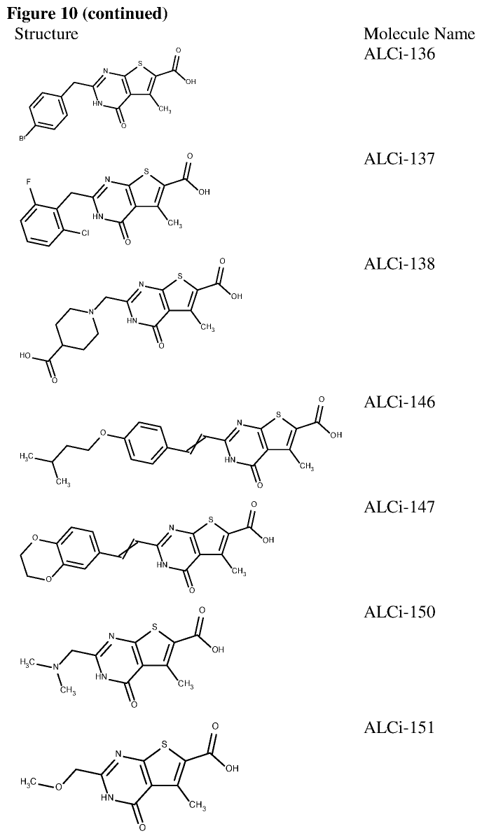

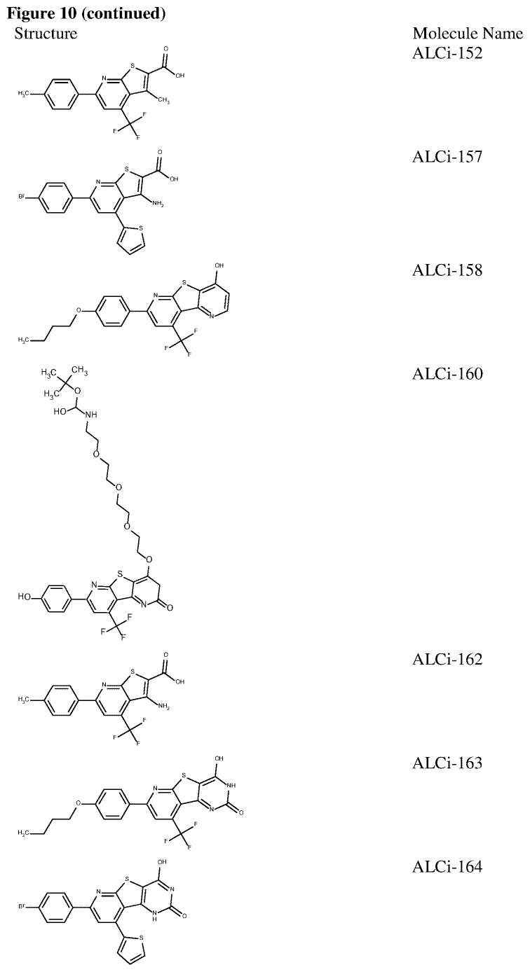

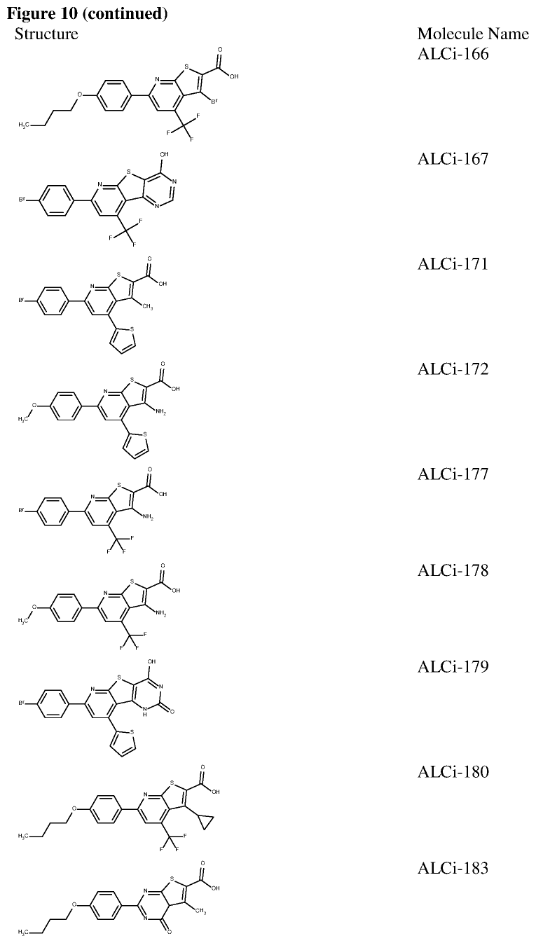

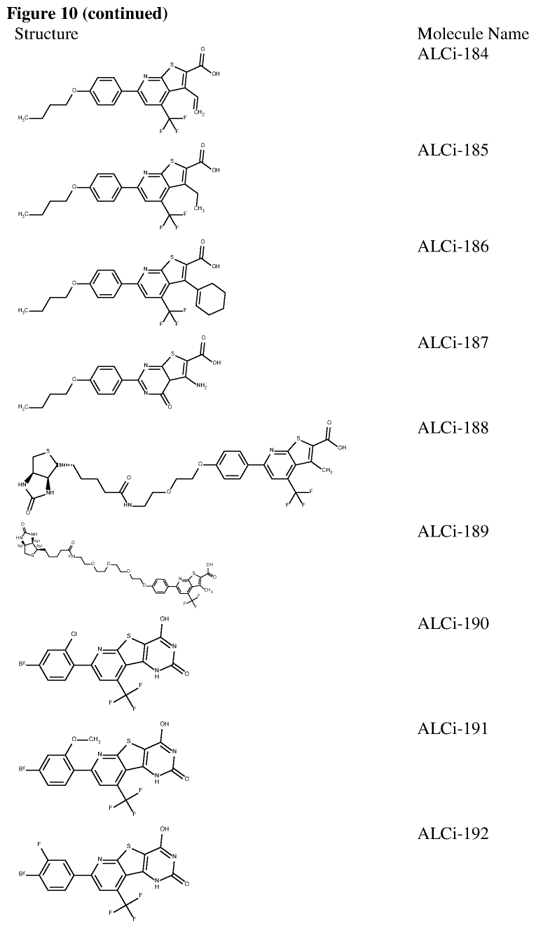

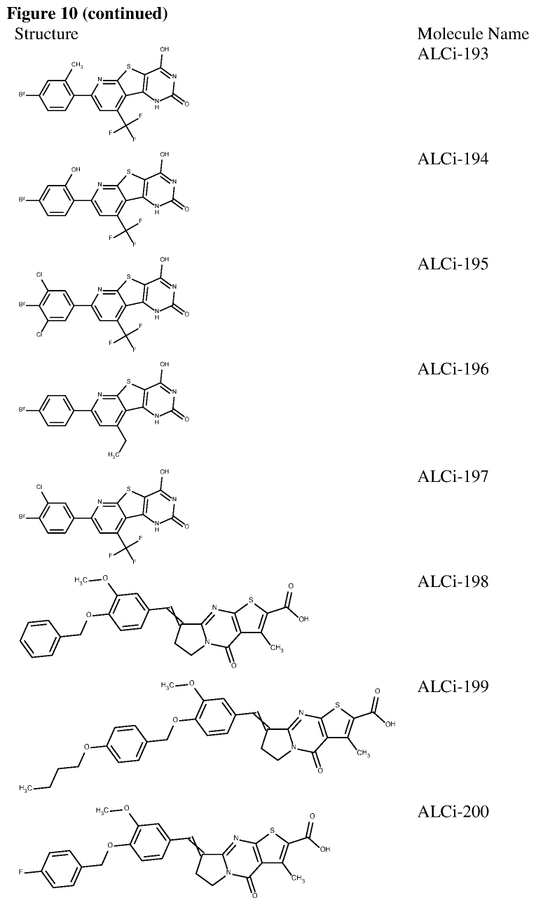

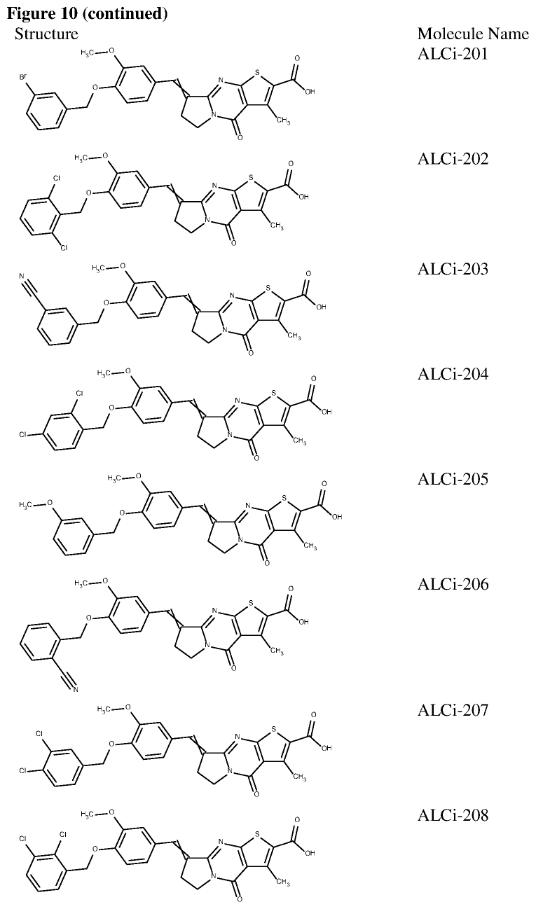

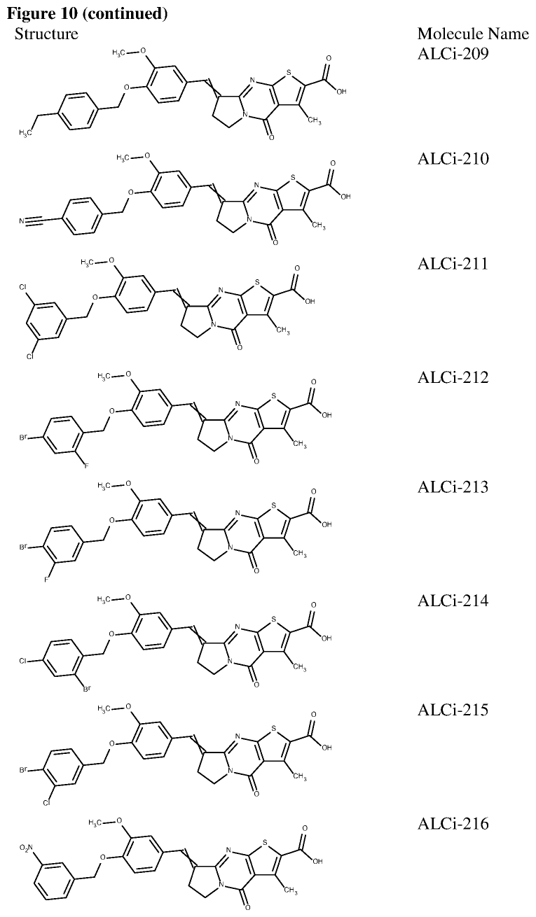

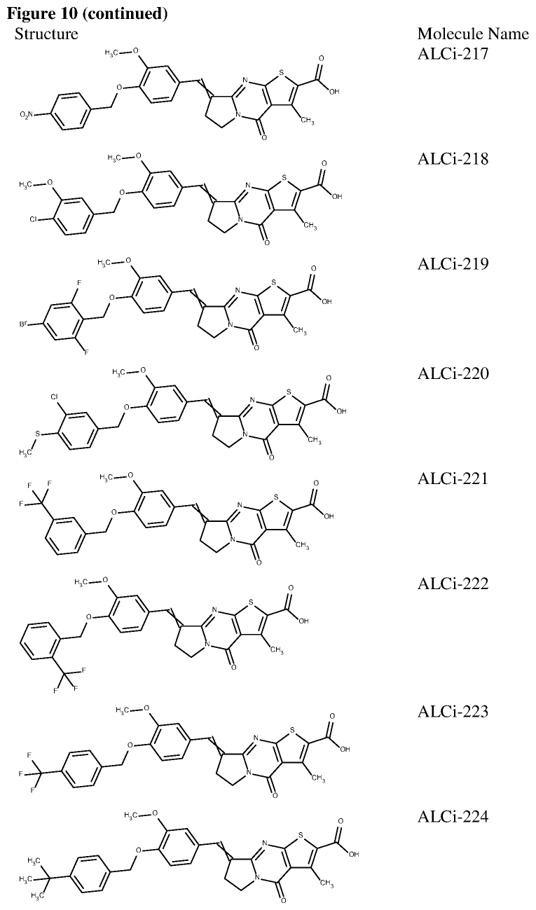

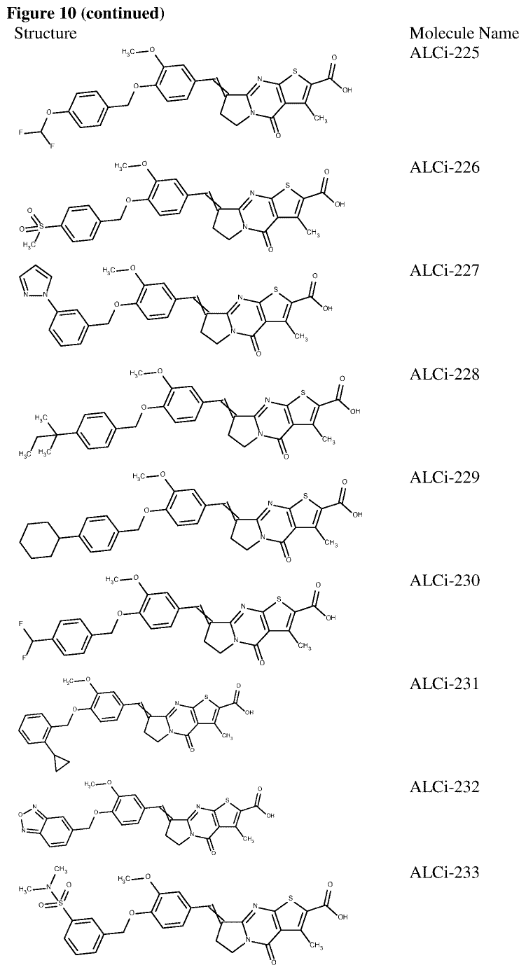

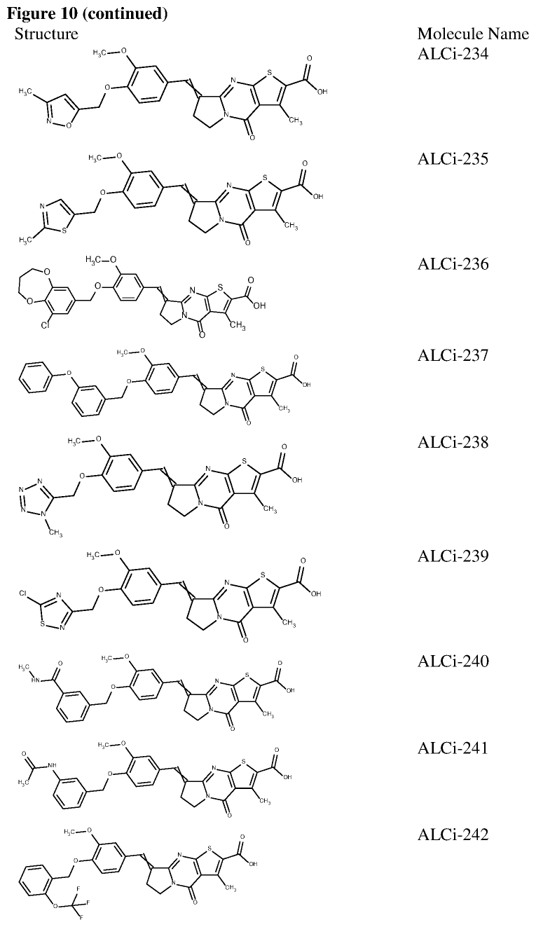

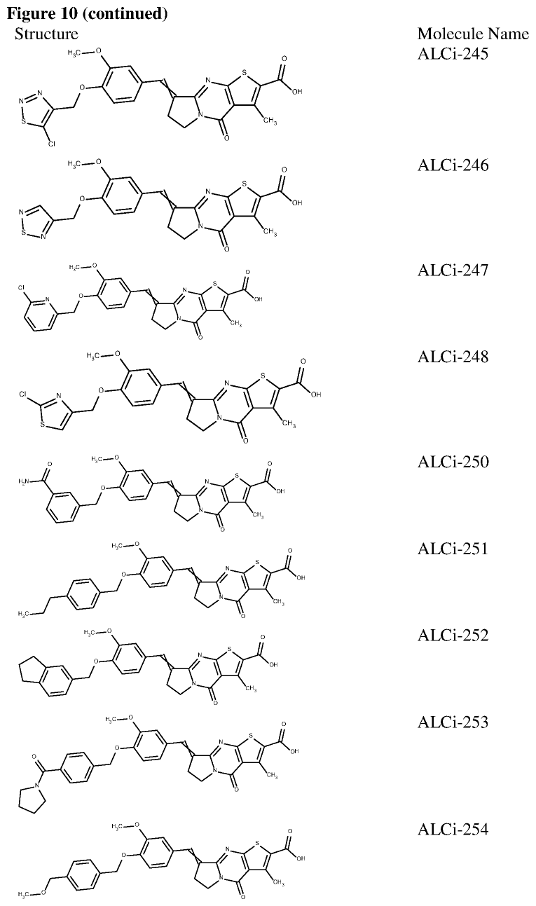

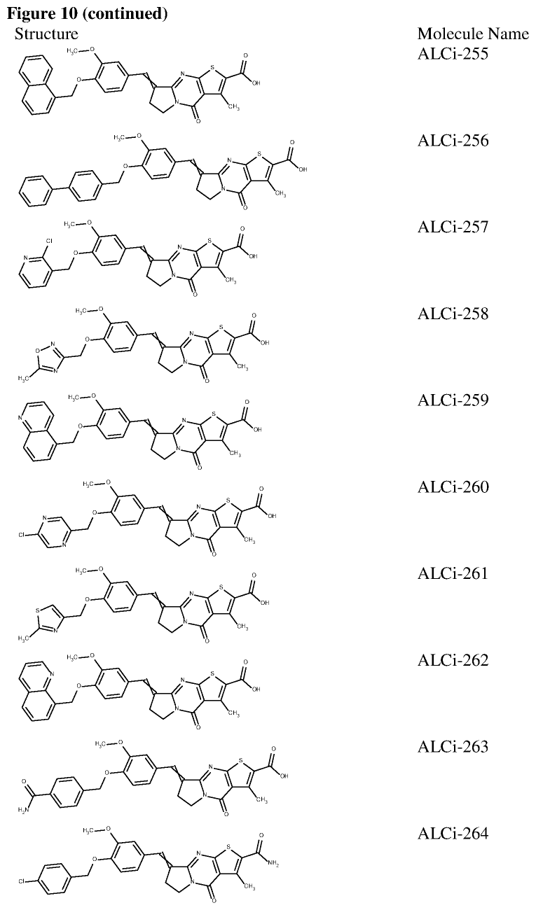

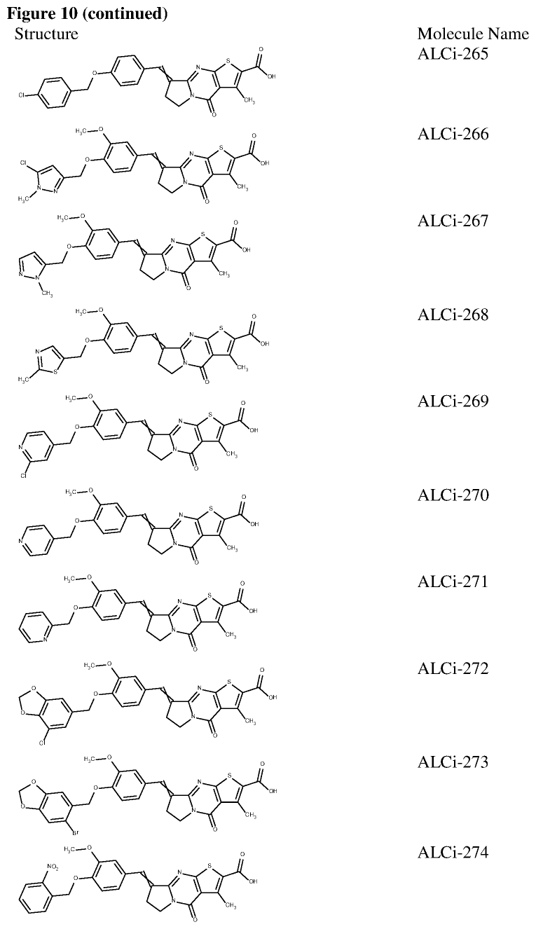

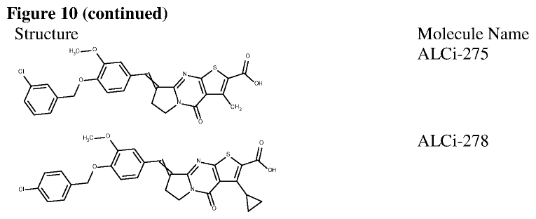

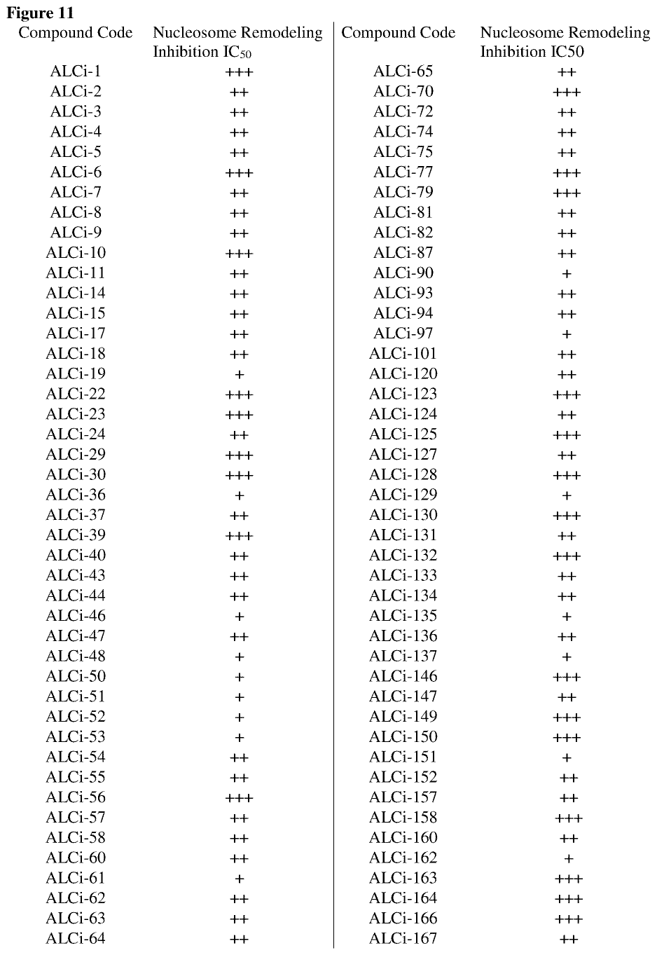

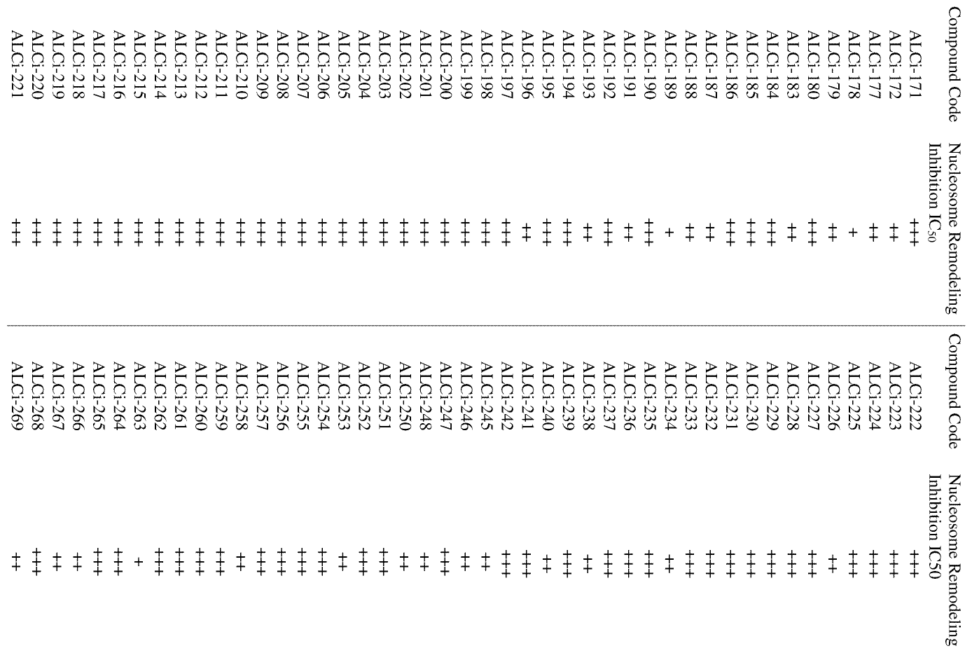

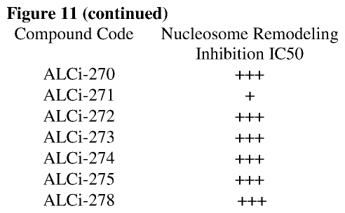

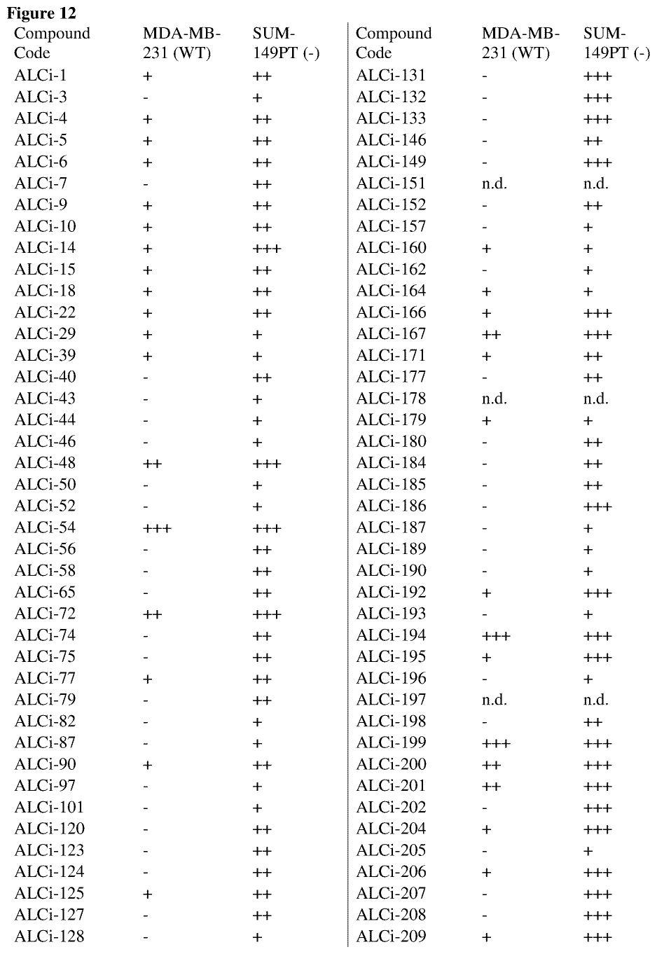

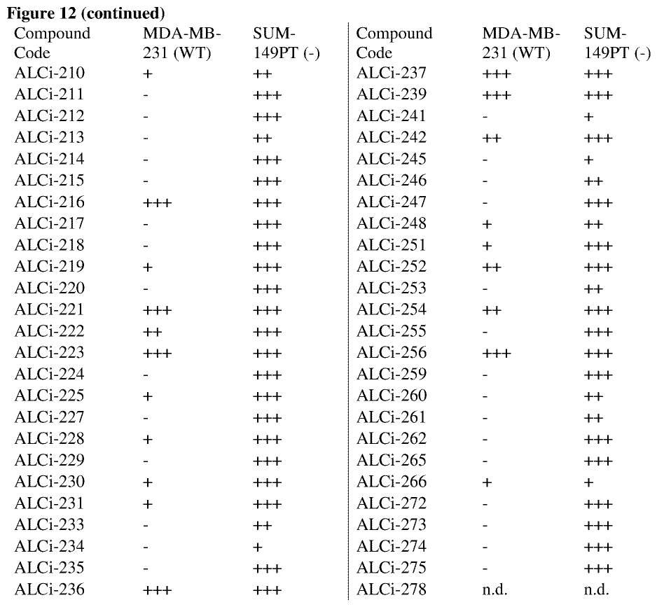

In a particularly preferred embodiment the compounds of the first and further aspect of the invention have the specific structures as indicated in FIG. 10.

The use of bifunctional compounds recruiting proteins involved in targeting proteins for degradation by the proteasome has emerged as a potential therapeutic strategy to degrade proteins that are involved in disease processes. This approach has met particular attention in cancer therapy (Khan S. et al., 2020 and Bushweller J H, 2019). Such bifunctional compounds are generally referred to as PROteolysis TArgeting Chimeras (PROTACs). The allosteric inhibitors of ALC1 of the first and further aspect of the invention specifically bind to ALC1 and are, thus suitable to recruit a protein that is part of the ubiquitination pathway to ALC1.

Accordingly, in a second aspect the present invention relates to a bifunctional compound comprising the allosteric inhibitor of ALC1 the first or further aspect of the present invention and a compound which recruits a protein that is part of the ubiquitination pathway to ALC1, preferably E3 ubiquitin ligase to ALC1 (E3 recruiter), wherein the allosteric inhibitor of ALC1 and the E3 recruiter are covalently linked, optionally through a linker. In the context of the bifunctional compounds of the present invention that ability of the allosteric inhibitors of ALC1 of the first and further aspect of the invention to specifically bind to ALC1 is of particular importance. It is, thus preferred that the allosteric inhibitors of ACL1 bind to full length human ALC1 with an amino acid sequence according to SEQ ID NO: 1 with a KD of 50 μM, more preferably of 10 μM or lower, more preferably of 5 μM or lower, even more preferably of 1 μM, more preferably of 500 nM, more preferably of 200 nM and even more preferably of 100 nM or lower.

The protein of the ubiquitination pathway may either be bound by a small molecule or a protein ligand, e.g. an antibody or antibody-like protein, that specifically binds to a protein of the ubiquitination pathway. Such protein ligands have been described, for example in U.S. Pat. No. 7,223,556 B1. Small molecules compounds that bind to a protein that is part of the ubiquitination pathway are well known in the art and can be used in the bifunctional compounds of the present invention. Examples of such molecules are described in EP 3 131 588, WO 2017/024317, U.S. Pat. Nos. 6,306,663, 7,041,298, US 2016/0176916, US 2016/0235730, US 2016/0235731, US 2016/0243247, WO 2016/105518, WO 2016/077380, WO 2016/105518, WO 2016/077375, WO 2017/007612, and WO 2017/024317.

The allosteric inhibitors of ALC1 is covalently linked to the compound which recruits a protein that is part of the ubiquitination pathway to ALC1. Preferably, the two components are covalently linked to each other through a linker. Suitable linkers have varying length and functionality. Preferably the linker is a carbon chain. In preferred embodiments, the carbon chain optionally comprises one, two, three, or more heteroatoms selected from N, O, and S. In preferred embodiments, the carbon chain comprises only saturated chain carbon atoms. In preferred embodiments, the carbon chain optionally comprises two or more unsaturated chain carbon atoms (e.g., C═C or C≡EC). In certain embodiments, one or more chain carbon atoms in the carbon chain are optionally substituted with one or more substituents, preferably oxo, C₁-C₆ alkyl, C₂-C₆ alkenyl, C₂-C₆ alkynyl, C₁-C₃ alkoxy, OH, halogen, NH₂, —NH(C₁-C₃ alkyl), —N(C₁-C₃ alkyl)₂, CN, C₃-C₇ cycloalkyl, heterocyclyl, phenyl, and heteroaryl).

In certain embodiments, the linker comprises at least 5 chain atoms (e.g., C, O, N, and S). In certain embodiments, the Linker comprises less than 20 chain atoms (e.g., C, O, N, and S). In certain embodiments, the Linker comprises 5, 6, 7, 8, 9, 10, 11, 12, 13, 14, 15, 16, 17, 18, or 19 chain atoms (e.g., C, O, N, and S). In certain embodiments, the Linker comprises 5, 7, 9, 11, 13, 15, 17, or 19 chain atoms (e.g., C, O, N, and S). In certain embodiments, the Linker comprises 5, 7, 9, or 11 chain atoms (e.g., C, O, N, and S). In certain embodiments, the Linker comprises 6, 8, 10, 12, 14, 16, or 18 chain atoms (e.g., C, O, N, and S). In certain embodiments, the Linker comprises 6, 8, 10, or 12 chain atoms (e.g., C, O, N, and S).

In certain embodiments, the Linker is a carbon chain optionally substituted with non-bulky substituents, preferably oxo, C₁-C₆ alkyl, C₂-C₆ alkenyl, C₂-C₆ alkynyl, C₁-C₃ alkoxy, OH, halogen, NH₂, —NH(C₁-C₃ alkyl), —N(C₁-C₃ alkyl)₂, CN, C₃-C₇ cycloalkyl, and CN).

In certain embodiments, the Linker is of Formula L(VI):

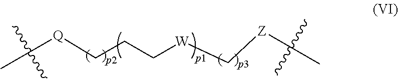

or an enantiomer, diastereomer, or stereoisomer thereof, wherein
p1 is an integer selected from 0 to 12;
p2 is an integer selected from 0 to 12;
p3 is an integer selected from 1 to 6;
each W is independently absent, CH₂, O, S, NH or NR₅;
Z is absent, CH₂, O, NH or NR₅;
each R₅ is independently H or C₁-C₃ alkyl, preferably C₁-C₃ alkyl; and
Q is absent or —CH₂C(0) NH—,
   wherein the linker is covalently bonded to the compound that compound which recruits a protein that is part of the ubiquitination pathway with the bond that is next to Q and to the allosteric inhibitor of ALC1 with the bond that is next to Z, and wherein the total number of chain atoms in the linker is less than 20.

In a third aspect the present inventions relates to a pharmaceutical composition comprising the allosteric inhibitor of ALC1 and a pharmaceutically acceptable excipient.

In a fourth aspect the present inventions relates to an ALC1 inhibitor (ALC1i) preferably those of the first aspect of the invention, for use in treating or ameliorating a proliferative disease in a patient, which comprises the administration of said ALC1i and optionally the administration of a Poly(ADP-ribose)-Polymerase inhibitor (PARPi). The present inventors have discovered that the PARP trapping activity of known PARPi's can be enhanced by ALC1i. Due to this surprising synergy the combined use of PARPi's and ALC1i's in the treatment of various proliferative diseases is particularly advantageous. This synergy is not limited to the allosteric inhibitors of the present invention, but will also be observed with inhibitors that inhibit ALCi by, e.g. binding to the ATPase site or by decreasing its expression, e.g. siRNAs directed at ALC1.

The ALC1 may be provided to the physician administering the antiproliferative therapy separately from the PARPi or in a kit of parts. Thus, in the embodiments in which both are to be combined to obtain the benefit of the synergy the ALC1 may be provided with instructions to combine it with a PARPi of alternatively in a fourth aspect the PARPi may be provided with instructions to combine it with a ALC1. Accordingly, in a fifth aspect the present invention relates to a PARPi for use in treating or ameliorating a proliferative disease in a patient, wherein the method comprises the administration of said PARPi and the administration of ALC1i.

In a preferred embodiment of the ALC1i for use of the fourth aspect of the invention or the PARPi for use of the fifth aspect of the invention the PARPi lowers PARP activity and/or inhibits PARP1, PARP2 and/or PARP3, preferably PARP2 on chromatin. The latter phenomenon is also referred to as PARP trapping. Thus, in a preferred embodiment PARP1, PARP2 and/or PARP3, preferably PARP2 is trapped.

In a preferred embodiment the ALC1i for use of the fourth aspect of the invention or the PARPi for use of the fifth aspect of the invention, the PARPi
(i) that lowers PARP activity is selected from small interfering RNA, and
(ii) that inhibits PARP1 is selected from the group consisting of a compound of
   (a) formula (II)

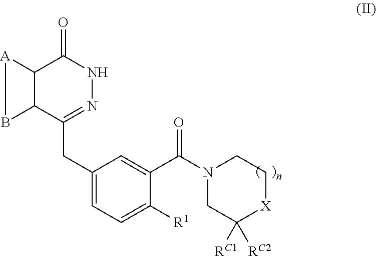

and isomers, salts, solvates, chemically protected forms, and prodrugs thereof wherein:
A and B together represent an optionally substituted, fused aromatic ring;
X is NR^{X} or CR^{X}R^{Y};
if X=NR″ then n is 1 or 2 and if X=CR^{X}R^{Y} then n is 1;
R^{X} is selected from the group consisting of H, optionally substituted C₁₋₂₀ alkyl, C₅₋₂₀ aryl, C₃₋₂₀ heterocyclyl, amido, thioamido, ester, acyl, and sulfonyl groups;
R^{Y} is selected from H, hydroxy, amino;
or R^{X} and R^{Y} may together form a spiro-C₃₋₇ cycloalkyl or heterocyclyl group;
R^{C1} and R^{C2} are independently selected from the group consisting of hydrogen and C₁₋₄ alkyl or when
   X is CR R^{Y}, R^{C1}, R^{C2}, R^{X} and R^{Y}, together with the carbon atoms to which they are attached, may form an optionally substituted fused aromatic ring; and
R₁ is selected from H and halo;
and
(b) formula (III)

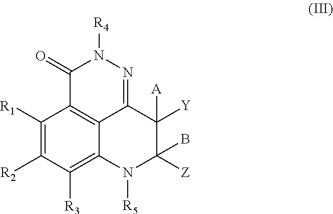

and isomers, salts, solvates, chemically protected forms, and prodrugs thereof wherein:
Y and Z are each independently selected from the group consisting of:
1. an aryl group optionally substituted with 1, 2, or 3 R₆;
2. a heteroaryl group optionally substituted with 1, 2, or 3 R₆;
3. a substituent independently selected from the group consisting of hydrogen, alkenyl (e.g. C₂₋₆-alkenyl), alkoxy (e.g. C₁₋₆-alkoxy), alkoxyalkyl (e.g. C₁₋₆-alkoxy-C₁₋₆-alkyl), alkoxycarbonyl (e.g. C₁₋₆-alkoxy-carbonyl), alkoxycarbonylalkyl (e.g. C₁₋₆-alkoxy-carbonyl-C₁₋₆-alkyl), alkyl (e.g. C₁₋₆-alkyl), alkynyl (e.g. C₂₋₆-alkynyl), arylalkyl (e.g. aryl-C₁₋₆-alkyl), cycloalkyl (e.g. C₃-8-cycloalkyl), cycloalkylalkyl (e.g. C₃₋₈-cycloalkyl-C₁₋₆-alkyl), haloalkyl (e.g. C₁₋₆-haloalkyl), hydroxyalkylene (e.g. hydroxy-C₁₋₆-alkylene), oxo, heterocycloalkyl (e.g. C₂₋₈-hetero cycloalkyl), heterocycloalkylalkyl (e.g. C₂₋₈-heterocycloalkyl-C₁₋₆-alkyl), alkylcarbonyl (e.g. C₁₋₆-alkyl-carbonyl), arylcarbonyl, heteroarylcarbonyl, alkylsulfonyl (e.g. C₁₋₆-alkyl-sulfonyl), arylsulfonyl, heteroarylsulfonyl, (R_{A}R_{B})alkylene (e.g. (R_{A}R_{B})—C₁₋₆-alkylene), (NR_{A}R_{B})carbonyl, (NR_{A}R_{B})carbonylalkylene (e.g. NR_{A}R_{B})carbonyl-C₁₋₆-alkylene), (NR_{A}R_{B})sulfonyl, and (R_{A}R_{B})sulfonylalkylene (e.g. (R_{A}R_{B})sulfonyl-C₁₋₆-alkylene);
wherein each R₆ is selected from OH, NO₂, CN, Br, Cl, F, I, C₁₋₆-alkyl, C₃₋₈-cycloalkyl, C₂₋₈-heterocycloalkyl; C₂₋₆-alkenyl, alkoxy (e.g. C₁₋₆-alkoxy), alkoxyalkyl (e.g. C₁₋₆-alkoxy-C₁₋₆-alkyl), alkoxycarbonyl (e.g. C₁₋₆-alkoxy-carbonyl), alkoxycarbonylalkyl (e.g. C₁₋₆-alkoxy-carbonyl-C₁₋₆-alkyl), C₂₋₆-alkynyl, aryl, arylalkyl (e.g. aryl-C₁₋₆-alkyl), C₃₋₈-cycloalkylalkyl (e.g. C₃₋₈-cycloalkyl-C₁₋₆-alkyl, haloalkoxy (e.g. C₁₋₆-haloalkoxy), haloalkyl (e.g. C₁₋₆-haloalkyl), hydroxyalkylene (e.g. hydroxy-C₁₋₆-alkylene), oxo, heteroaryl, heteroarylalkoxy (e.g. heteroaryl-C₁₋₆-alkoxy), heteroaryloxy, heteroarylthio, heteroarylalkylthio (e.g. heteroaryl-C₁₋₆-alkylthio), heterocycloalkoxy (e.g. C₂₋₈-heterocycloalkoxy), C₂₋₈-heterocycloalkylthio, heterocyclooxy, heterocyclothio, NR_{A}R_{B}, (R_{A}R_{B})C₁₋₆-alkylene, (NR_{A}R_{B})carbonyl, (R_{A}R_{B})carbonylalkylene (e.g. R_{A}R_{B})carbonyl-C₁₋₆-alkylene), (NR_{A}R_{B})sulfonyl, and (NR_{A}R_{B})sulfonylalkylene (e.g. (NR_{A}R_{B})sulfonyl-C₁₋₆-alkylene);
R₁, R₂, and R₃ are each independently selected from the group consisting of hydrogen, halogen, alkenyl (e.g. C₂₋₆-alkenyl), alkoxy (e.g. C₁₋₆-alkoxy), alkoxycarbonyl (e.g. C₁₋₆-alkoxy-carbonyl), alkyl (e.g. C₁₋₆-alkyl), cycloalkyl (e.g. C₃₋₈-cycloalkyl), alkynyl (e.g. C₂₋₆-alkynyl), cyano, haloalkoxy (e.g. C₁₋₆-haloalkoxy), haloalkyl (e.g. C₁₋₆-haloalkyl), hydroxyl, hydroxyalkylene (e.g. hydroxy-C₁₋₆-alkylene), nitro, NR_{A}R_{B}, NR_{A}R_{B} alkylene (e.g. NR_{A}R_{B} C₁₋₆-alkylene), and (R_{A}R_{B})carbonyl;
A and B are each independently selected from hydrogen, Br, Cl, F, I, OH, C₁₋₆-alkyl, C₃₋₈-cycloalkyl, alkoxy (e.g. C₁₋₆-alkoxy), alkoxyalkyl (e.g. C₁₋₆-alkoxy-C₁₋₆-alkyl), wherein C₁₋₆-alkyl, C₃₋₈-cycloalkyl, alkoxy, alkoxyalkyl are optionally substituted with at least one substituent selected from OH, NO₂, CN, Br, Cl, F, I, C₁₋₆-alkyl, and C₃₋₈-cycloalkyl, wherein B is not OH;
R_{A}, and R_{B} are independently selected from the group consisting of hydrogen, alkyl (e.g. C₁₋₆-alkyl), cycloalkyl (e.g. C₃₋₈-cycloalkyl), and alkylcarbonyl (e.g. C₁₋₆-alkyl-carbonyl); or RA and RB taken together with the atom to which they are attached form a 3-10 membered heterocycle ring optionally having one to three heteroatoms or hetero functionalities selected from the group consisting
   of —O—, —NH, —N(C₁₋₆-alkyl)-, —NCO(C₁₋₆-alkyl)-, —N(aryl)-, —N(aryl-C₁₋₆-alkyl-), —N(substituted-aryl-C₁₋₆-alkyl-)—, —N(heteroaryl)-, —N(heteroaryl-C₁-C₆-alkyl-)—, —N(substituted-heteroaryl-C₁₋₆ alkyl-)—, and —S— or S(O)q-, wherein q is 1 or 2 and the 3-10 membered heterocycle ring is optionally substituted with one or more substituents;
R₄ and R₅ are each independently selected from the group consisting of hydrogen, alkyl (e.g. C₁₋₆-alkyl), cycloalkyl (e.g. C₃₋₈-cycloalkyl), alkoxyalkyl (e.g. C₁₋₆-alkoxy-C₁₋₆-alkyl), haloalkyl (e.g. C₁₋₆-haloalkyl), hydroxyalkylene (e.g. hydroxy-C₁₋₆-alkylene), and (NR_{A}R_{B})alkylene (e.g. NR_{A}R_{B} C₁₋₆-alkylene);
(iii) that inhibits PARP1 and PARP2 is selected from the group consisting of a compound of
   (a) formula (IV)

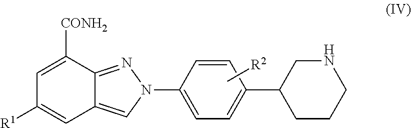

and isomers, salts, solvates, chemically protected forms, and prodrugs thereof wherein:
R₁ is hydrogen or fluorine; and
R₂ is hydrogen or fluorine;
and
(b) formula (V)

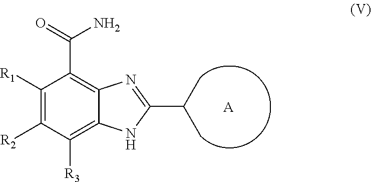

and isomers, salts, solvates, chemically protected forms, and prodrugs thereof wherein:
R₁, R₂, and R₃ are independently selected from the group consisting of hydrogen, alkenyl (e.g. C₁₋₆-alkenyl), alkoxy (e.g. C₁₋₆-alkoxy), alkoxycarbonyl (e.g. C₁₋₆-alkoxycarbonyl), alkyl (e.g. C₁₋₆-alkyl), alkynyl (e.g. C₁₋₆-alkynyl), cyano, haloalkoxy (e.g. C₁₋₆-haloalkoxy), haloalkyl (e.g. C₁₋₆-haloalkyl), halogen, hydroxy, hydroxyalkyl (e.g. C₁₋₆-hydroxyalkyl), nitro, NR_{A}R_{B}, and (NR_{A}R_{B})carbonyl;
A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl (e.g. C₁₋₆-alkenyl), alkoxy (e.g. C₁₋₆-alkoxy), alkoxyalkyl (e.g. C₁₋₆-alkoxy-C₁₋₆-alkyl), alkoxycarbonyl (e.g. C₁₋₆-alkoxycarbonyl), alkoxycarbonylalkyl (e.g. C₁₋₆-alkoxycarbonyl-C₁₋₆-alkyl), alkyl (e.g. C₁₋₆-alkyl), alkynyl (e.g. C₁₋₆-alkynyl), aryl, arylalkyl (e.g. aryl-C₁₋₆-alkyl), cycloalkyl (e.g. C₃₋₈-cycloalkyl), cycloalkylalkyl (e.g. C₃₋₈-cycloalkyl-C₁₋₆-alkyl), cyano, haloalkoxy (e.g. C₁₋₆-haloalkoxy), haloalkyl (e.g. C₁₋₆-haloalkyl), halogen, heterocycle, heterocyclealkyl (e.g. heterocycle-C₁₋₆-alkyl), heteroaryl, heteroarylalkyl (e.g. heteroaryl-C₁₋₆-alkyl), hydroxy, hydroxyalkyl (e.g. C₁₋₆-hydroxyalkyl), nitro, NR_{C}R_{D}, (NR_{C}R_{D})alkyl (e.g. (NR_{C}R_{D})—C₁₋₆-alkyl), (NR_{C}R_{D})carbonyl, (NR_{C}R_{D})carbonylalkyl (e.g. (NR_{C}R_{D})carbonyl-C₁₋₆-alkyl), and (NR_{C}R_{D})sulfonyl; and
R_{A}, R_{B}, R_{C}, and R_{D} are independently selected from the group consisting of hydrogen, alkyl (e.g. C₁₋₆-alkyl), and alkycarbonyl (e.g C₁₋₆-alkylcarbonyl).
(iv) that inhibits PARP1, PARP2 and PARP3 is a compound of formula (VI)

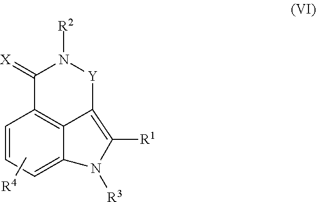

and isomers, salts, solvates, chemically protected forms, and prodrugs thereof wherein:
R₁ is: H; halogen; cyano; an optionally substituted alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from halogen, hydroxy, nitro, and amino, alkoxy (e.g. C₁₋₆-alkoxy), alkyl (e.g. C₁₋₆-alkyl), and aryl groups unsubstituted or substituted with one or more substituents selected from halogen, hydroxy, nitro, carboxy, and optionally substituted amino and ether groups (such as O-aryl)); or —C(O)—R₁₀, where R₁₀ is: H; an optionally substituted alkyl (e.g. C₁₋₆-alkyl), alkenyl (e.g. C₁₋₆-alkenyl), alkynyl (e.g. C₁₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from halogen, hydroxy, nitro, amino, and alkyl (e.g. C₁₋₆-alkyl) and aryl groups unsubstituted or substituted with one or more substituents selected from halo, hydroxy, nitro, and amino); or OR₁₀₀ or NR₁₀₀R₁₁₀, where R₁₀₀ and R₁₁₀ are each independently H or an optionally substituted alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, and heteroaryl groups unsubstituted or substituted with one or more substituents selected from halogen, hydroxy, nitro, amino, and alkyl (e.g. C₁₋₆-alkyl) and aryl groups unsubstituted or substituted with one or more substituents selected from halogen, hydroxy, nitro, and optionally substituted amino groups);
R₂ is H or alkyl (e.g. C₁₋₆-alkyl);
R₃ is H or alkyl (e.g. C₁₋₆-alkyl);
R₄ is H, halogen or alkyl (e.g. C₁₋₆-alkyl);
X is O or S;
Y is (CR₅R₆)(CR₇R₈)ₙ or N—C(R₅), where:
n is 0 or 1;
R₅ and R₆ are each independently H or an optionally substituted alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from halogen, hydroxy, nitro, amino, and lower alkyl (e.g. C₁₋₄-alkyl), lower alkoxy (e.g. C₁₋₄-alkoxy), or aryl groups unsubstituted or substituted with one or more substituents selected from halogen, hydroxy, nitro, and amino); and
R₇ and R₅ are each independently H or an optionally substituted alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from halogen, hydroxy, nitro, amino, and lower alkyl (e.g. C₁₋₄-alkyl), lower alkoxy (e.g. C₁₋₄-alkoxy), and aryl groups unsubstituted or substituted with one or more substituents selected from halogen, hydroxy, nitro, and amino);
where when R₁, R₄, R₅, R₆, and R₇ are each H, R₈ is not unsubstituted phenyl.

In a preferred embodiment the ALC1i for use of the fourth aspect of the invention or the PARPi for use of the fifth aspect of the invention the PARPi is selected from the group consisting of Olaparib, Talazoparib, Niraparib, Rucaparib, and Veliparib, in particular of Veliparib, Olaparib, and Talazoparib.

In a preferred embodiment the ALC1i for use of the fourth aspect of the invention or the PARPi for use of the fifth aspect of the invention the ALC1i is a direct inhibitor of the ATPase activity of ALC1, or is an allosteric inhibitor of ALC1.

In a preferred embodiment the ALC1i for use of fourth aspect of the invention or the PARPi for use of the fifth aspect of the invention the ALC1i is the inhibitor of ALC1 of any of the first or further aspect of the invention or the bifunctional compound of the second aspect.

In a preferred embodiment the ALC1i for use of the fourth aspect of the invention or the PARPi for use of the fifth aspect of the invention the proliferative disease is selected from a BRCA1 and/or 2-deficient tumor, a tumor in which expression of PARP1, PARP2, PARP3 and/or ALC1 is increased in comparison to non-tumor cells.

In a preferred embodiment the ALC1i for use of the fourth aspect of the invention or the PARPi for use of the fifth aspect of the invention the tumor disease is selected from hepato cellular carcinoma, breast cancer, ovarian cancer, prostate cancer, and colorectal cancer.

In a preferred embodiment the ALC1i for use of the fourth aspect of the invention or the PARPi for use of the fifth aspect of the invention (i) the ALC1i potentiates the cancer-cell killing efficacy of the PARPi, (ii) a reduced amount of PARPi is administered, and/or (iii) PARPi resistance is bypassed.

In a preferred embodiment the ALC1i for use of the fourth aspect of the invention or the PARPi for use of the fifth aspect of the invention the PARPi and ALC1i are administered concomitantly or separately.

In a sixth aspect the present invention relates to a kit of parts comprising separately packaged a PARPi and an ALC1i or a composition comprising a PARPi and an ALC1i, preferably with instructions for use to treat or ameliorate a proliferative disease.

### EXPERIMENTAL SECTION

### Cell Lines Used

A preferred cell line used in the context of the examples is the osteosarcoma cell line termed U2OS. The U2OS cell line is a human cancer cell line that was established from a 15-year-old, Caucasian female in 1964 by J. Ponten and E. Saksela from a moderately differentiated sarcoma of the tibia. (U-2 OS ATCC® HTB-96™ Homo sapiens bone osteosarcoma, 2016) It is available from numerous sources including ATCC® HTB-96®.

As a BRCA wild-type cell line, MDA-MB-231 cells were used. The cells were established from an aneuploid female human. The cells were extracted from the mammary gland (breast) in the metastatic site as a pleural effusion. (MDA-MB-231 (ATCC® HTB-26™ Homo sapiens epithelial mammary gland) It is available from numerous sources including ATCC® HTB-26™.

As a BRCA negative cell line, SUM-149-PT cells were used. The cell line is a triple negative breast cancer (TNBC) cell line, derived from primary human invasive ductual carcinoma metastatic nodule from a 40 year old female. It contains a hemizygous BRCA1 mutation (p.Pro724Leufs*12) and is available from numerous sources including bioIVT.

### PARP-2 Trapping:

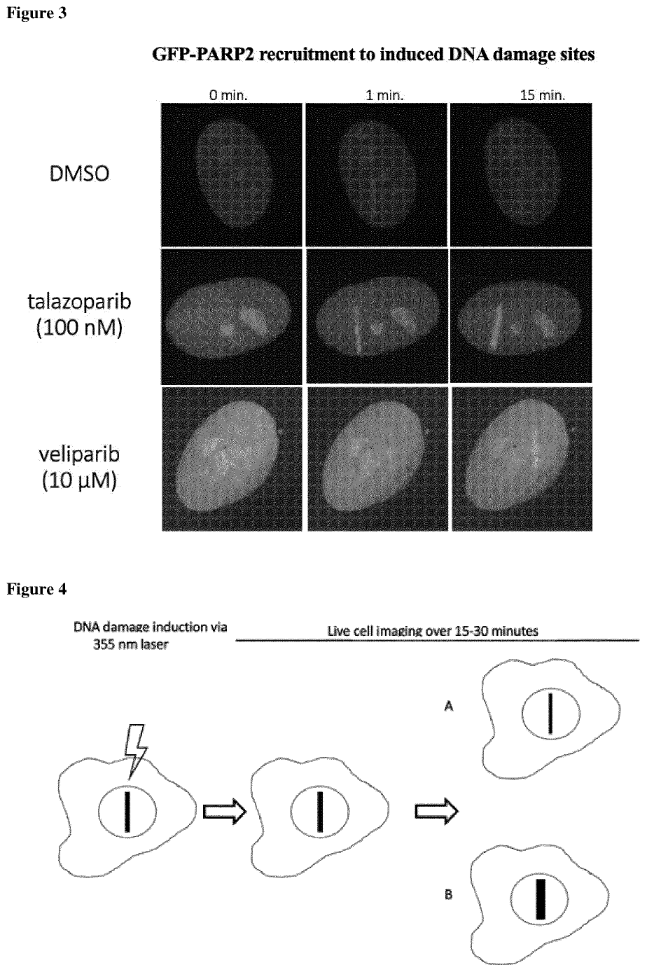

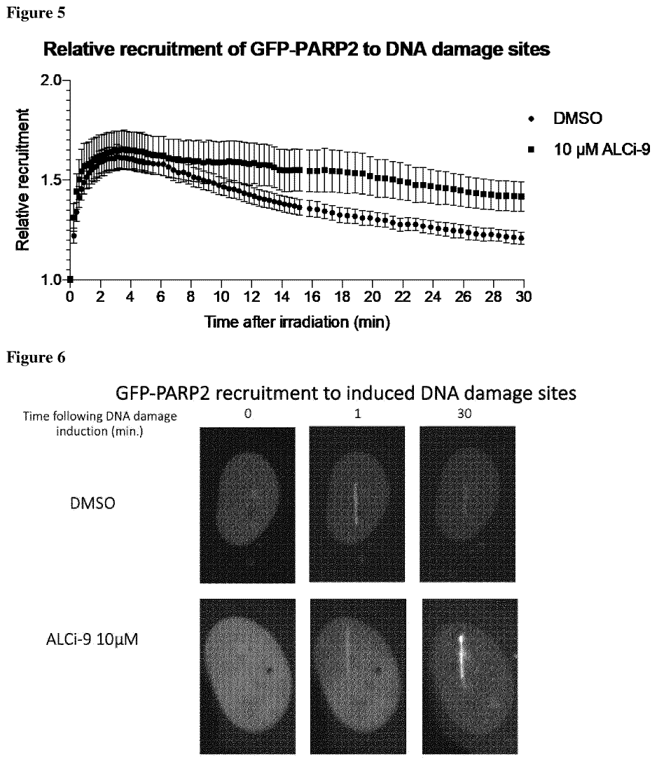

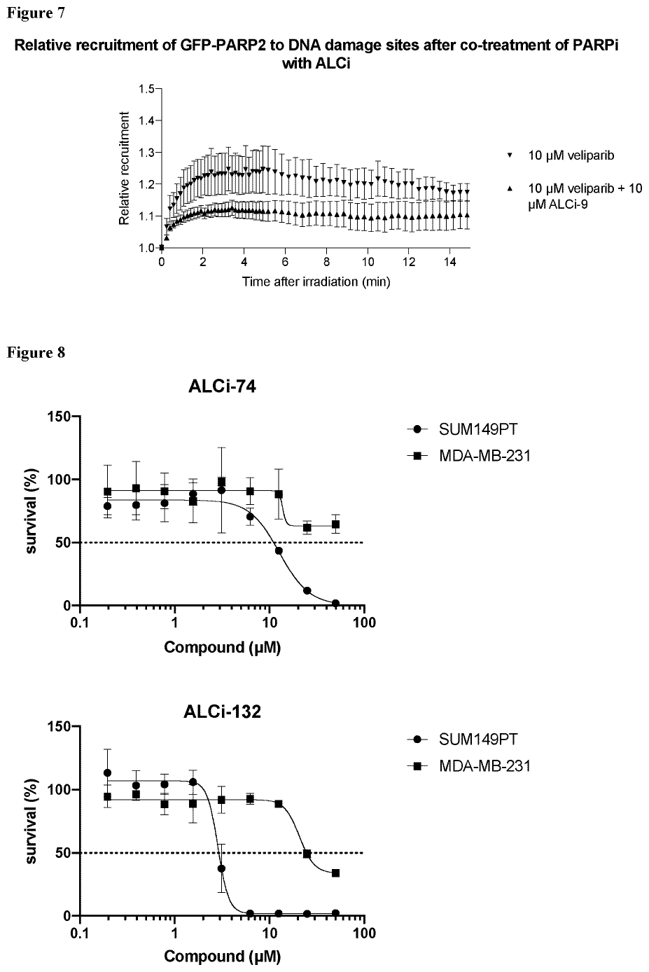

U2OS cells were seeded onto 4-well Nunc Lab-Tek chambers (Thermo Fisher Scientific) in normal DMEM. Cells were cultured over night at 37° C. and transfected with a GFP-tagged PARP2 plasmid using Lipofectamine. 24 h after transfection, the cells were imaged using the Zeiss AxioObserver Z1 confocal spinning-disk microscope equipped with a sCMOS ORCA Flash 4.0 camera (Hamamatsu). Live-cell imaging experiments were performed with C-Apo 63× water immersion objective lens. During this time, the cells were maintained in Leibovitz's L-15 media (Gibco), supplemented with 10% FBS, at 37° C. in the absence of CO₂. For PARP-2 trapping imaging and analysis, cells with comparable transgene expression levels were selected. DNA damage was induced along a line of 88 pixels that is exposed for 400 msec with 20% laser power of a 355 nm laser operated through a single-point scanning head (UGA-42 firefly, Rapp OptoElectronics). Schematic of live-cell PARP-2 trapping assay was shown in FIG. 4.

The accumulation of fluorophore-tagged proteins at laser micro-irradiation sites was followed for 15-30 minutes. The cells were treated for 1 hour with the indicated inhibitor concentrations at 37° C. prior to experimental analysis. As a control, cells were treated with corresponding concentrations of DMSO. The accumulation of fluorophore-tagged proteins at micro-irradiation sites was quantified using a custom-made macro in Fiji/ImageJ. The damage region of interest was selected, the mean fluorescent intensity of the nucleus was determined, and the background signal was subtracted. The recruitment was calculated via the following formula: (damage region (t)−background signal (t))/(nucleus intensity (t)−background (t))

Inhibitor Vs. Response (Cell Survival) Assay

For validation of the small molecule inhibitors, the synthetic lethality of BRCA and ALC1 was addressed using MDA-MB-231 cells as a BRCA wild-type cell line and SUM-149-PT as a BRCA1 deficient cell line. Cells were seeded in 96-well plates (5000 cells/well) and treated with titrations of ALC1 inhibitors starting at 50 μM. As a control for “no-treatment”, DMSO was added to the cells. The cells were cultured at 37° C., CO₂ 5% for 5 days until they were fixed with 10% TCA for 1 h and stained with sulforhodamine dye for 30 minutes. After washing the cells with 1% Acetic Acid, 10 mM Tris (pH 10.5) solution was used to solubilize the stained cells. The absorbance was measured at 492 nm using the SUNRISE TECAN, the data were normalized to the number of cells at timepoint 0 and to 100% survival (=DMSO control) and analyzed using GraphPad Prism. Survival curves were fitted using “Inhibitor vs. response curves with variable slope (four parameters)”. The IC50 values for treatment with ALC1 inhibitors are shown in FIG. 8.

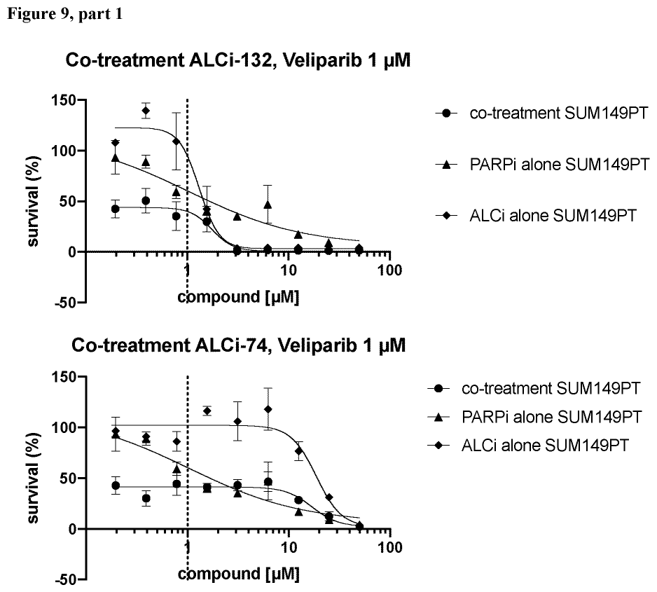

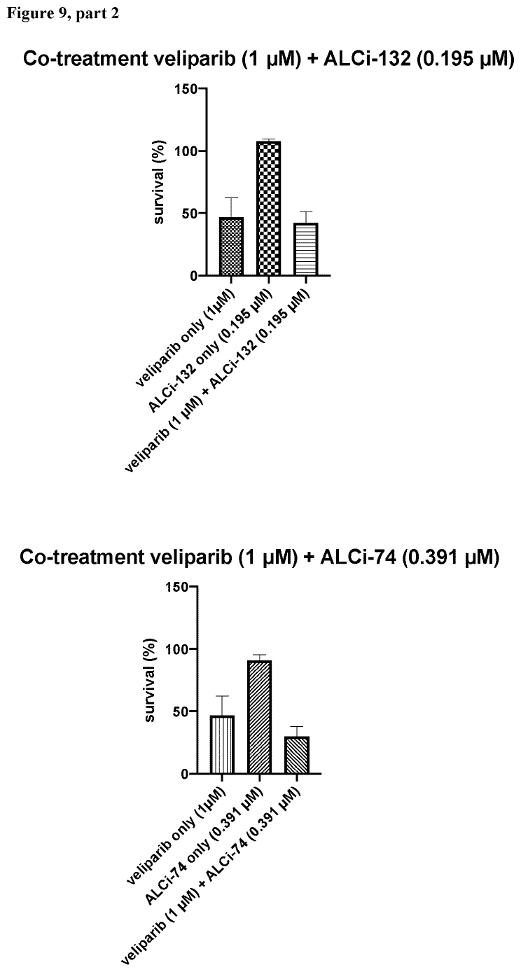

For further validation of the compounds, colony formation assays were applied. Here, less cells were seeded for the assay (100 cells/well). The cells were treated with ALC1 inhibitor or with a combination of PARPi and an ALC1 inhibitor for 11 days. The cells were fixed and data was analyzed as mentioned above. Results for treatment with ALC1 inhibitors are shown in FIGS. 8 and 9.

### FRET-Based Nucleosome Sliding Assay

This assay utilizes mid-positioned mononucleosomes that allow for monitoring the sliding activity of the ALC1 remodeling enzyme using a FRET readout. Each nucleosome is labeled with two FRET dyes: the octamer is labeled with Cy5 (Cy5-maleimide coupling to H2B) and one of the DNA ends is labeled with Cy3. The DNA template includes the 147 bp 601 DNA positioning sequence flanked by DNA overhangs on each side. Other nucleosome positioning sequences, both artificial constructs and naturally occurring sequences, even if less efficient than the 601 sequence in positioning nucleosomes, can also be used. The nucleosomes are assembled by salt gradient dialysis using purified, Cy5-labeled histone octamers and purified, Cy3-labeled DNA templates to yield the FRET-labeled mid-positioned nucleosomes. These nucleosomes will start with low FRET and will have a low Cy5 fluorescence signal when excited with the Cy3 excitation maximum wavelength as the two fluorophores are too far apart for efficient FRET. As the ALC1 remodeling reaction proceeds and the remodeling enzymes slides the octamer towards the DNA end, the distance between the two FRET dyes decreases and the signal from Cy5/FRET increases. Hence, increase in FRET can be directly used as readout for sliding.

### DNA Template Construction for Mononucleosome Reconstitution

For reconstitution of mononucleosomes, the non-natural Widom 601 nucleosome positioning sequence was used as a high affinity binding site for the histone octamer (see Lowary, P. T. & Widom, J. New DNA sequence rules for high affinity binding to histone octamer and sequence-directed nucleosome positioning. J. Mol. Biol. 276, 19-42 (1998)). Cy3-labeled DNA containing these 601 sequences can be constructed using methods including PCR amplification, restriction digestion of DNA plasmids followed by a Klenow end labeling reaction or other standard molecular biological techniques.

### Preparation of ALC1 and Histone Octamers

Full length human ALC1 (1-897) or truncated versions thereof were expressed and purified as N-terminally 6× His-tagged fusion protein from E. coli as published before (Singh, H. R., et al., 2017).

Human histone proteins were recombinantly expressed in E. coli (either using classical IPTG induction or using autoinduction media) and purified from E. coli inclusion bodies. The purification scheme includes the extraction/solubilization of histones from inclusion bodies using guanidium chloride, followed by reverse phase chromatography. The purified histones were lyophilized resulting in TFA-salts of the purified histone proteins.

### Preparation of Nucleosomes

To assemble the nucleosomes, template DNA (250 μg/ml final concentration) was mixed with purified histone octamers in a high salt buffer at different molar ratios of histone octamers to DNA. The ionic strength of this mixture was then reduced <600 mM NaCl by continuous dialysis against a low salt buffer at 4° C. Finally, the material was dialyzed against TEA-20 (10 mM triethanolamine-Cl pH 7.5, 20 mM NaCl, 0.1 mM EDTA). The best molar ratio, i.e. the ratio that yields full assembly of the DNA into nucleosomes was picked and used for further assemblies.

Alternatively, the nucleosomes can be assembled by other methods such as deposition of histone octamers onto DNA using polyglutamate or histone chaperones, or by salt step dilution.

### Method of Measuring ALC1 Mediated Sliding: ALC1 Nucleosomal Sliding Assay

Sliding reactions were performed in 384 well plates at RT in 10 mM Tris-HCl, pH 8.1, 75 mM KCl, 1 mM MgCl₂, 1.0 mM EGTA, 10% glycerol, 0.5 mM dithiothreitol (DTT), 0.01% TritonX100, 0.02% NP40 and reaction mixtures contained mid-positioned nucleosome, tri-ADP ribose or (ADP-ribose), and ALC1 chromatin remodeling enzyme. Sliding was initiated by addition of ATP followed by shaking the plates for 5 sec at 1450 rpm and sealing them with a foil compatible with fluorescence reading. The FRET signal was immediately recorded using a fluorometer (BMG reader PheraStar FSX, channel A: excitation 520 nm, emission 680 nm; channel B: excitation 520 nm, emission 590 nm) and unless stated otherwise, remodeling proceeded for 30 min.

The FRET signal was calculated as the signal at 680 nm (emission of Cy5) divided by the signal at 590 nm (emission of Cy3) and multiplied by 10,000. To obtain an apparent rate of ALC1-mediated nucleosome sliding, the increase in FRET as a function over time was plotted and the initial velocities of ALC1-mediated nucleosome sliding were obtained by fitting the resulting kinetic trace by a linear curve fit.

High Throughput Screening (HTS) and IC50 Determination for Molecules that Modulate ALC1-Mediated Sliding

For High Throughput Screening (HTS) and IC₅₀ determination for compounds that modulate the sliding activity of ALC1, the nucleosome was incubated for 30 min as described above with ALC1 and triADP-ribose or (ADP-ribose), in the presence of the putative ALCi prior to initiating sliding by ATP addition. Then, the rate of sliding (initial velocity) was determined as described above and compared against the rate of sliding in the absence of the putative modulator/compound (% inhibition). For IC₅₀ determination, the observed % inhibition (y-axis) was plotted against compound concentrations (x-axis) using GraphPad Prism and fitted using a nonlinear regression model (four parameters).

### In Silico Modeling of ALC1 Inhibitors

In order to identify the binding pocket of the small molecule inhibitors of ALC1, a homology model of the ALC1 helicase domain was built for use in molecular docking and molecular dynamics simulations.

The homology model and all ligands were then prepared for molecular docking and flexible docking was then preformed into the homology model on an Intel® Xeon® Platinum 8268 CPU @ 2.90 GHz cpu ALCi-22 was chosen to be run in MD simulations due to its low IC50 in biochemical sliding assays and docking pose, which was in a previously unidentified pocket within the ATPase domain but not the ATP binding site itself. MD was then initiated on an NVIDIA Tesla V100-SXM2-32gb GPU. After the MD simulation was complete, every 10th frame was taken from the trajectory. These frames were aligned to the initial pose and PCA was carried out. Plots of the PCA were generated using the first two principle components. 8 clusters of protein conformations were identified manually. A random point near the center of each cluster was extracted as PDB files. These frames were inspected manually and frame number 33449405 was eventually selected for use in figures due to the ligand proximity to a catalytic residue in the ALC1 ATP binding site as well as its occupancy of a previously undescribed binding pocket. This pocket is composed of the following amino acids of human ALC1: L101, Y153, C156, L157, A160, L163, K164, V173, D174, E175, A176, H177, R178, L179, S183, L186, H187, T189, L190, F193, L200, L201, T202, N208, S209, E212, L213, L216, F219.

### SAR Analysis of ALC1 Inhibitors

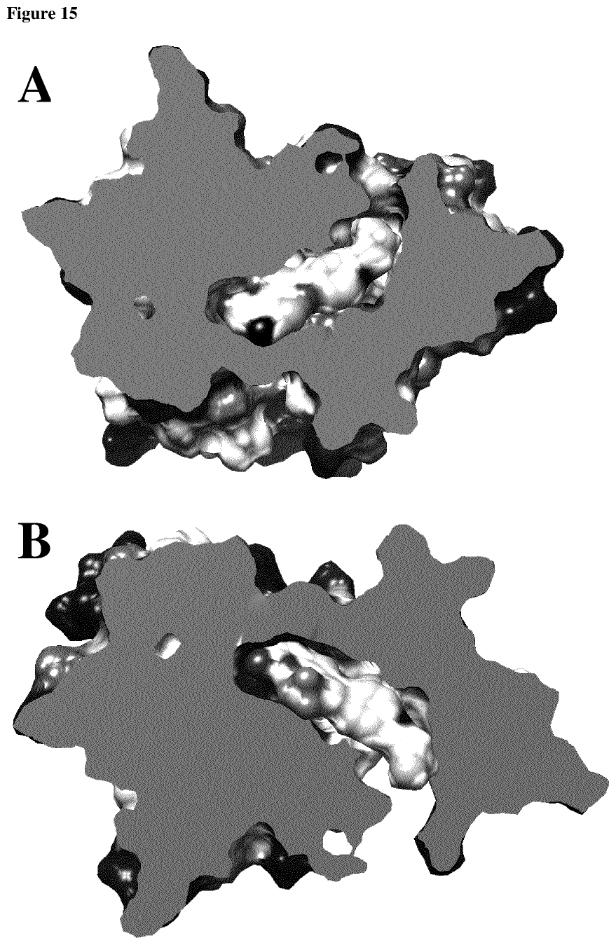

Examining the biochemical and cellular data, several clear trends emerge relating to the structure and activity of the ligands. The “tail” of the ligands clearly show that long, hydrophobic attachments are preferred with a terminal cyclopentane (ALCi-123 vs ALCi-125) or without (ALCi-4 vs ALCi-2, vs ALCi-1, vs ALCi-22). Additionally, hydrophilic substituents in the “tail” region are highly detrimental (ALCi-135). This is in line with the modeling pose which shows that the “tail” of the molecule is positioned in the highly hydrophobic region of the binding pocket (FIGS. 14 and 15). Considering the pyridine ring, it appears that a CF₃ substitution opposite the N is beneficial over a CF₂H substitution (ALCi-8 vs ALCi-4), but a thiophene ring is favored over a CF₃ (ALCi-4 vs ALCi-56). This is again explained well by the modeling pose, which shows this region of the molecule is again interacting with a highly hydrophobic region of the binding pocket (FIGS. 14 and 15). Additionally, in the “head” region of the inhibitors, molecules with more hydrophilic substituents are favored over those with fewer (ALCi-6 vs ALCi-72). Additionally, it appears that the highest potency is achieved when a carboxylic acid is substituted in the “head” region (ALCi-132), which is predicted to interact directly with ARG135 and potentially ASN 165 (FIG. 19). In fact, it appears that interaction with ARG165 may be critical for inhibition or binding as ALCi-31, ALCi-32, ALCi-33, and ALCi-34, which lack corresponding hydrophilic groups at the head region, are all inactive (data not shown). Taken together, it is clear that the SAR of the compounds supports the binding site prediction in the novel binding pocket by in-silico modeling.

### Synthesis of Selected ALC1 Inhibitors

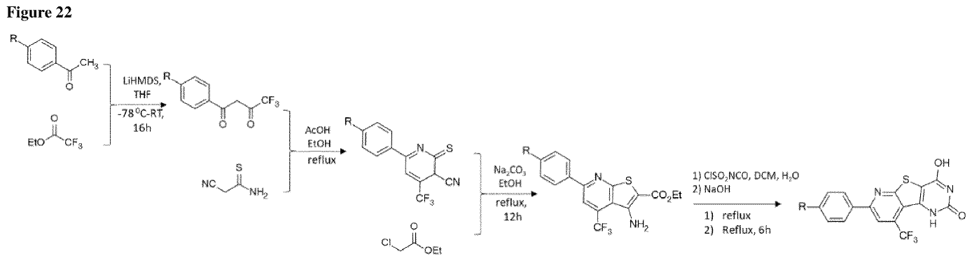

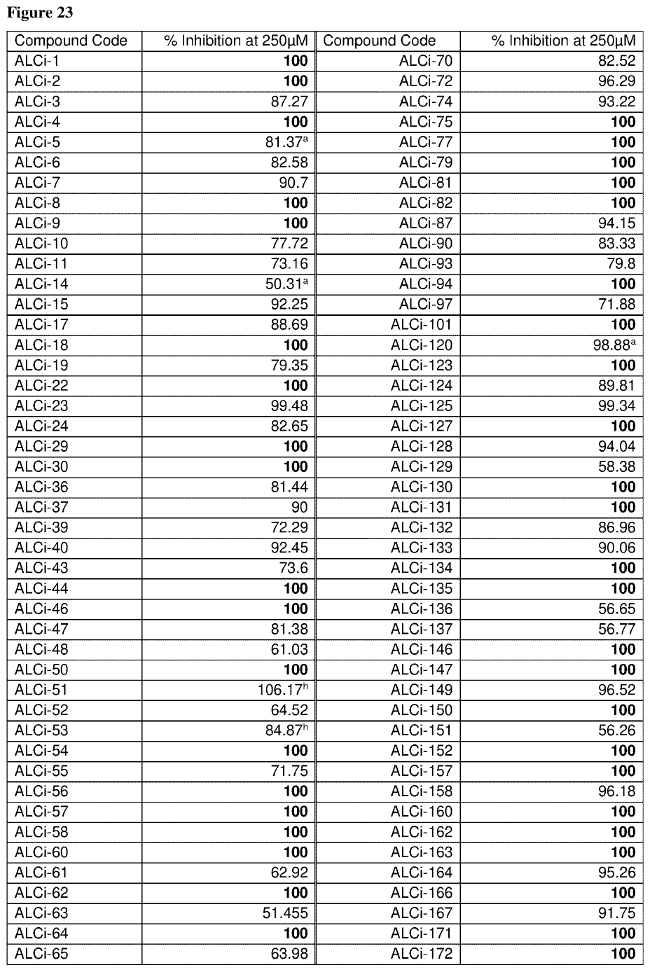

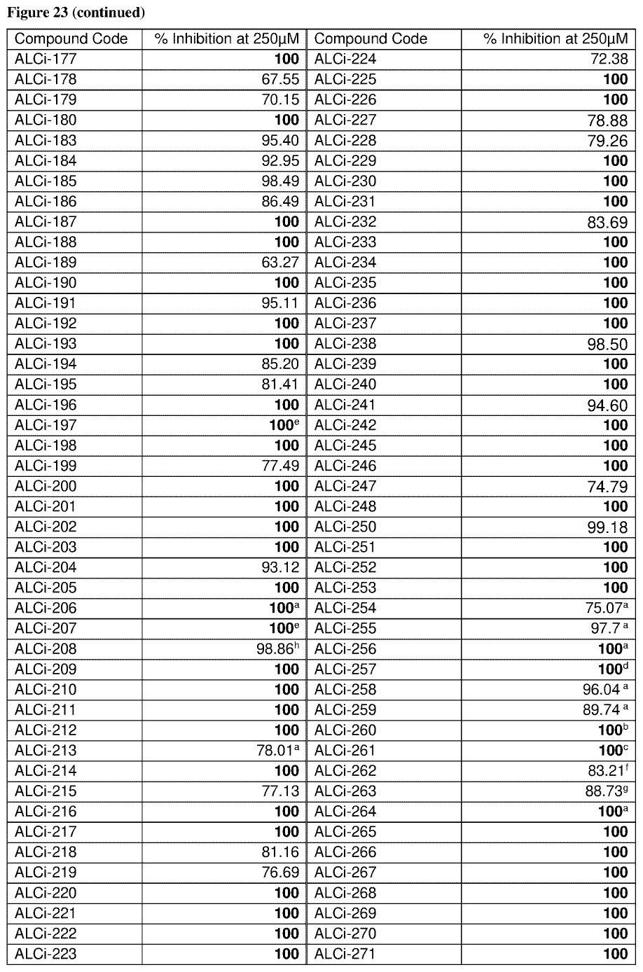

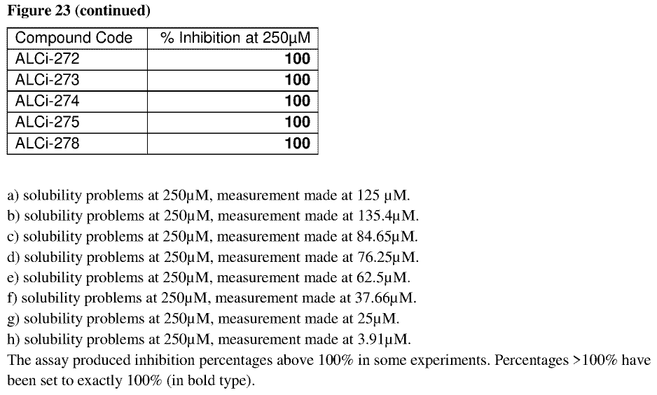

The synthesis of ALCi-72 begins with an Aldol condensation between 1-(4-bromophenyl) ethan-1-one and ethyl 2,2,2-trifluoroacetate with LiHMDS as base in THF at a temperature from −78° C. to room temperature over 16 hours, yielding 1-(4-bromophenyl)-4,4,4-trifluorobutane-1,3-dione (see FIG. 22, R=Br). This product then participates in pyridine formation with 2-cyanoethanethioamide in acetic acid and ethanol under reflux yielding 6-(4-bromophenyl)-2-sulfanylidene-4-(trifluoromethyl)-2,3-dihydropyridine-3-carbonitrile. In step 3, 6-(4-bromophenyl)-2-sulfanylidene-4-(trifluoromethyl)-2,3-dihydropyridine-3-carbonitrile undergoes a cyclization to thiophenopyridine with ethyl 2-chloroacetate with sodium carbonate as base in ethanol under reflux conditions over 12 hours, which yields ethyl 3-amino-6-(4-bromophenyl)-4-(trifluoromethyl) thieno[2,3-b]pyridine-2-carboxylate. Finally, in step 5, ethyl 3-amino-6-(4-bromophenyl)-4-(trifluoromethyl) thieno[2,3-b]pyridine-2-carboxylate undergoes urea formation and cyclization into the final product, ALCi-72, by means of CISO₂NCO, in DCM and water under reflux and then sodium hydroxide under reflux for 6 hours respectively.

ALCi-117 synthesis starts with an Aldol condensation between 1-(4-butoxyphenyl) ethan-1-one and ethyl 2,2,2-trifluoroacetate with NaH a base in THF from 0° C. to room temperature over 16 hours to produce 1-(4-butoxyphenyl)-4,4,4-trifluorobutane-1,3-dione. This product then participates in a pyridine formation with 2-cyanoethanethioamide in acetic acid and ethanol, yielding 6-(4-butoxyphenyl)-2-sulfanyl-4-(trifluoromethyl)pyridine-3-carbonitrile. In step 3, 6-(4-butoxyphenyl)-2-sulfanyl-4-(trifluoromethyl)pyridine-3-carbonitrile undergoes a cyclization to thiophenopyridine with ethyl 2-chloroacetate with sodium carbonate as base in ethanol under reflux conditions, which yields ethyl 3-amino-6-(4-butoxyphenyl)-4-(trifluoromethyl) thieno[2,3-b]pyridine-2-carboxylate. Step 4 is a Sandmeyer reaction with ethyl 3-amino-6-(4-butoxyphenyl)-4-(trifluoromethyl) thieno[2,3-b]pyridine-2-carboxylate, yielding ethyl 3-bromo-6-(4-butoxyphenyl)-4-(trifluoromethyl) thieno[2,3-b]pyridine-2-carboxylate. Subsequently, ethyl 3-bromo-6-(4-butoxyphenyl)-4-(trifluoromethyl) thieno[2,3-b]pyridine-2-carboxy late is incubated with CH₃BF₃—K+, Cs₂CO₃, Pd (dppf) Cl₂, DCM, and dioxane at 150° C. for 30 minutes in a Suzuki coupling reaction to yield ethyl 6-(4-butoxyphenyl)-3-methyl-4-(trifluoromethyl) thieno[2,3-b]pyridine-2-carboxylate. Finally, step 6 is a hydrolysis of ethyl 6-(4-butoxyphenyl)-3-methyl-4-(trifluoromethyl) thieno[2,3-b]pyridine-2-carboxylate with sodium hydroxide to yield the produce, ALCi-117.

ALCi-132 synthesis begins with the synthesis of the intermediate 4-[(4-chlorophenyl) methoxy]-3-methoxybenzaldehyde to be used in the final step via a 1-(bromomethyl)-4-chlorobenzene between 4-hydroxy-3-methoxybenzaldehyde and 1-(bromomethyl)-4-chlorobenzene under reflux conditions with acetone and potassium carbonate. Step 1 of the synthesis is a thiophene formation employing ethyl 3-oxobutanoate, ethyl 2-cyanoacetate and S8 in ethanol and Et₂NH to form 2,4-diethyl 5-amino-3-methylthiophene-2,4-dicarboxylate, 2,4-diethyl 5-amino-3-methylthiophene-2,4-dicarboxylate then participates in an amide formation with oxolane-2,5-dione, in a mixture of ether, benzene and dioxane at room temperature to form 3-{[3-carbamoyl-5-(ethoxycarbonyl)-4-methylthiophen-2-yl]carbamoyl}propanoic acid. In step 3, 3-{[3-carbamoyl-5-(ethoxycarbonyl)-4-methylthiophen-2-yl]carbamoyl}propanoic acid undergoes a Steglich esterification to form ethyl 4-carbamoyl-5-(4-methoxy-4-oxobutanamido)-3-methylthiophene-2-carboxylate. ethyl 4-carbamoyl-5-(4-methoxy-4-oxobutanamido)-3-methylthiophene-2-carboxylate then undergoes a Zn(BH₄)₂ reduction reaction to form ethyl 4-carbamoyl-5-(4-hydroxybutanamido)-3-methylthiophene-2-carboxylate, which is then cyclized using sodium hydroxide under reflux conditions for 16 hours to form 2-(3-hydroxypropyl)-5-methyl-4-oxo-4H,4aH-thieno[2,3-d]pyrimidine-6-carboxylic acid, 2-(3-hydroxypropyl)-5-methyl-4-oxo-4H,4aH-thieno[2,3-d]pyrimidine-6-carboxylic acid is then esterified using EtOH with H₂SO₄ under reflux for 16 hours, yielding ethyl 2-(3-hydroxypropyl)-5-methyl-4-oxo-4H,4aH-thieno[2,3-d]pyrimidine-6-carboxylate. A Mitsunobu reaction is utilized at room temperature to convert ethyl 2-(3-hydroxypropyl)-5-methyl-4-oxo-4H,4aH-thieno[2,3-d]pyrimidine-6-carboxy late to ethyl 4-methyl-2-oxo-6-thia-1λ⁴,8-diazatricyclo[7.3.0.0^{3,7}]dodeca-1 (9),4,7-triene-5-carboxylate, which finally undergoes an aldol condensation with the intermediate 4-[(4-chlorophenyl) methoxy]-3-methoxybenzaldehyde with Ac₂O under reflux to produce ALCi-132.

### LIST OF REFERENCES

Abbott, J. M., Zhou, Q., Esquer, H., Pike, L., Broneske, T. P., Rinaldetti, S., Abraham, A. D., Ramirez, D. A., Lunghofer, P. J., Pitts, T. M., Regan, D. P., Tan, A. C., Gustafson, D. L., Messersmith, W. A. & LaBarbera, D. V. (2020). First-in-Class Inhibitors of Oncogenic CHD1L with Preclinical Activity against Colorectal Cancer. Molecular Cancer Therapeutics, 19 (8), 1598-1612. https://doi.org/10.1158/1535-7163.mct-20-0106.
Ahel, D., Horejsi, Z., Wiechens, N., Polo, S. E., Garcia-Wilson, E., Ahel, I., Flynn, H., Skehel, M., West, S. C., Jackson, S. P., et al. (2009). Poly(ADP-ribose)-dependent regulation of DNA repair by the chromatin remodeling enzyme ALC1. Science. 325, 1240-1243.Cheng, W., Su, Y., and Xu, F. (2013). CHD1L: a novel oncogene. Mol. Cancer 12, 170.
Bushweller J H. (2019) Targeting transcription factors in cancer—from undruggable to reality, NatuRe Reviews, 19:611-624.
Charifson, P S.; Kuntz, I D. Recent successes and continuing limitations in computer aided drug design. In: Charifson, P S., editor. Practical Application of Computer Aided Drug Design. New York: Dekker; 1997.
Flaus A., Martin D M A, Barton G J, Owen-Hughes T (2006), Identification of multiple distinct Snf2 subfamilies with conserved structural motifs Nucleic Acids Res.; 34 (10): 2887-2905.
Greer J, Erickson J W, Baldwin J J, Varney M D. Application of the three-dimensional structures of protein target molecules in structure-based drug design. J Med Chem 1994; 37:1035-1047.
Gottschalk, A. J., Timinszky, G., Kong, S. E., Jin, J., Cai, Y., Swanson, S. K., Washburn, M. P., Florens, L., Ladurner, A. G., Conaway, J. W., et al. (2009). Poly(ADP-ribosyl)ation directs recruitment and activation of an ATP-dependent chromatin remodeler. Proc. Natl. Acad. Sci. 106, 13770-13774.
Hazuda D J, Anthony N J, Gomez R P, Jolly S M, Wai J S, Zhuang L, Fisher T E, Embrey M, Guare J P Jr, Egbertson M S, et al. A nap-thyridine carboxamide provides evidence for discordant resistance between mechanistically identical inhibitors of HIV-1 integrase. Proc Natl Acad Sci USA 2004; 101:11233-11238
Jorgensen W L. The many roles of computation in drug discovery. Science 2004; 303:1813-1818.
Khan S, Yonghan He X, Zhang X, Yuan Y, Pu S, Kong Q, Zheng G, and Zhou D (2020) PROteolysis TArgeting Chimeras (PROTACs) as emerging anticancer therapeutics Oncogene (2020) 39:4909-4924.
Lehmann, L. C., Hewitt, G., Aibara, S., Leitner, A., Marklund, E., Maslen, S. L., Maturi, V., Chen, Y., van der Spoel, D., Skehel, J. M., et al. (2017). Mechanistic insights into autoinhibition of the oncogenic chromatin remodeler ALC1. Mol. Cell 68, 847-859.
Li, Y., He, L. R., Gao, Y., Zhou, N. N., Liu, Y., Zhou, X. K., Liu, J. F., Guan, X. Y., Ma, N. F., and Xie, D. (2019). CHD1L contributes to cisplatin resistance by upregulating the ABCB1-NF-κB axis in human non-small-cell lung cancer. Cell Death Dis. 10, 1-17.
Liverton N J, Holloway M K, McCauley J A, Rudd M T, Butcher J W, Carroll S S, DiMuzio J, Fandozzi C, Gilbert K F, Mao S S, et al. Molecular modeling based approach to potent P2-P4 macrocyclic inhibitors.
Lord, C. J., and Ashworth, A. (2012). The DNA damage response and cancer therapy. Nature 481, 287-294.
Luijsterburg, M. S., de Krijger, I., Wiegant, W. W., Shah, R. G., Smeenk, G., de Groot, A. J. L., Pines, A., Vertegaal, A. C. O., Jacobs, J. J. L., Shah, G. M., et al. (2016). PARP1 links CHD2-mediated chromatin expansion and H3.3 deposition to DNA repair by non-homologous endjoining. Mol. Cell 61, 547-562.
Mehrotra, P. V., Ahel, D., Ryan, D. P., Weston, R., Wiechens, N., Kraehenbuehl, R., Owen-Hughes, T., and Ahel, I. (2011). DNA repair factor APLF is a histone chaperone. Mol. Cell 41, 46-55.
Murai, J., Huang, S.-N., Das, B. B., Renaud, A., Zhang, Y., Doroshow, J. H., Ji, J., Takeda, S. & Pommier, Y. (2012). Trapping of PARP1 and PARP2 by Clinical PARP Inhibitors. Cancer Research, 72(21), 5588-5599. https://doi.org/10.1158/0008-5472.can-12-2753
Murai, J., Huang, S.-Y. N., Renaud, A., Zhang, Y., Ji, J., Takeda, S., Morris, J., Teicher, B., Doroshow, J. H., and Pommier, Y. (2014). Stereospecific PARP trapping by BMN 673 and comparison with olaparib and rucaparib. Mol. Cancer Ther. 13, 433-443
Ray Chaudhuri, A., and Nussenzweig, A. (2017). The multifaceted roles of PARP1 in DNA repair and chromatin remodelling. Nat. Rev. Mol. Cell Biol. 18, 610-621.
Sellou, H., Lebeaupin, T., Chapuis, C., Smith, R., Hegele, A., Singh, H. R., Kozlowski, M., Bultmann, S., Ladurner, A. G., Timinszky, G., et al. (2016). The poly(ADP-ribose)-dependent chromatin remodeler A1c1 induces local chromatin relaxation upon DNA damage. Mol. Biol. Cell 27, 3791-3799.
Singh, H. R., Nardozza, A. P., Möller, I. R., Knobloch, G., Kistemaker, H. A. V., Hassler, M., Harrer, N., Blessing, C., Eustermann, S., Kotthoff, C., et al. (2017a). A poly-ADP-ribose trigger releases the auto-inhibition of a chromatin remodeling oncogene. Mol. Cell 68, 860-871.
Smeenk, G., Wiegant, W. W., Marteijn, J. A., Luijsterburg, M. S., Sroczynski, N., Costelloe, T.,
Romeijn, R. J., Pastink, A., Mailand, N., Vermeulen, W., et al. (2013). Poly(ADP-ribosyl) ation links the chromatin remodeler SMARCA5/SNF2H to RNF168-dependent DNA damage signaling. J. Cell Sci. 126, 889-903.
Su, F. R., Ding, J. H., Bo, L., and Liu, X. G. (2014). Chromodomain helicase/ATPase DNA binding protein 1-like protein expression predicts poor prognosis in nasopharyngeal carcinoma. Exp. Ther. Med. 8, 1745-1750.
Timinszky, G., Till, S., Hassa, P. O., Hothorn, M., Kustatscher, G., Nijmeijer, B., Colombelli, J., Altmeyer, M., Stelzer, E. H. K., Scheffzek, K., et al. (2009). A macrodomain-containing histone rearranges chromatin upon sensing PARP1 activation. Nat. Struct. Mol. Biol. 16, 923-929.
U-2 O S ATCC® HTB-96T M Homo sapiens bone osteosarcoma. (2016). ATCC https://www.lgcstandards-atcc.org/products/all/HTB-96.aspx?geo_country=de#characteristics
Schames J R, Henchman R H, Siegel J S, Sotriffer C A, Ni H, McCammon J A. Discovery of a novel binding trench in HIV integrase. J Med Chem 2004; 47:1879-1881.Salomon-Ferrer, R., Case, D. A. and Walker, R. C. (2013) ‘An Overview of the Amber Biomolecular Simulation Package’, WIREs Comput. Mol. Sci., 3, pp. 198-210.
Zandarashvili, L., Langelier, M.-F., Velagapudi, U. K., Hancock, M. A., Steffen, J. D., Billur, R., Hannan, Z. M., Wicks, A. J., Krastev, D. B., Pettitt, S. J., Lord, C. J., Talele, T. T., Pascal, J. M. & Black, B. E. (2020). Structural basis for allosteric PARP-1 retention on DNA breaks. Science, 368 (6486), eaax6367. https://doi.org/10.1126/science.aax6367
Karras, G. I., Kustatscher, G., Buhecha, H. R., Allen, M. D., Pugieux, C., Sait, F., Bycroft, M. & Ladurner, A. G. (2005). The macro domain is an ADP-ribose binding module. The EMBO Journal, 24 (11), 1911-1920. https://doi.org/10.1038/sj.emboj.7600664
Zimmermann, M., Murina, O., Reijns, M. A. M., Agathanggelou, A., Challis, R., Tarnauskaitė, Ž. ė., Muir, M., Fluteau, A., Aregger, M., McEwan, A., Yuan, W., Clarke, M., Lambros, M. B., Paneesha, S., Moss, P., Chandrashekhar, M., Angers, S., Moffat, J., Brunton, V. G., . . . . Durocher, D. (2018). CRISPR screens identify genomic ribonucleotides as a source of PARP-trapping lesions. Nature, 559(7713), 285-289. https://doi.org/10.1038/s41586-018-0291-z.
von Itzstein M, Wu W Y, Kok G B, Pegg M S, Dyason J C, Jin B, Van Phan T, Smythe M L, White H F, Oliver S W, et al. Rational design of potent sialidase-based inhibitors of influenza virus replication. Nature 1993; 363:418-423.

1-15. (canceled)

16. An allosteric inhibitor of Chromodomain-helicase-DNA-binding protein 1-like (ALC1), wherein the allosteric inhibitor has a structure selected of formula (VII): 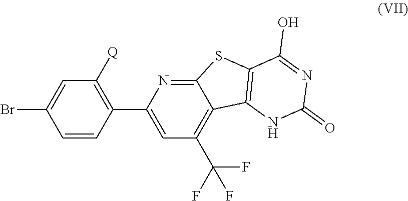
wherein Q is H or OH; and
isomers, salts, solvates, chemically protected forms, and prodrugs thereof.

17. A pharmaceutical composition comprising the allosteric inhibitor of ALC1 (ALC1i) of claim 16 and a pharmaceutically acceptable excipient.

18. A method for treating or ameliorating a proliferative disease in a patient, wherein the method comprises the administration of the ALC1i of claim 16.

19. A method of treating or ameliorating a proliferative disease in a patient, wherein the method comprises the administration of a PARPi and the administration of the ALC1i of claim 16.

20. The method of claim 19, wherein the PARPi lowers PARP activity and/or inhibits PARP1, PARP2 and/or PARP3, preferably PARP2 on chromatin, wherein preferably the PARPi
(i) that lowers PARP activity is selected from small interfering RNA, and
(ii) that inhibits PARP1 is selected from the group consisting of a compound of
(a) formula (II) 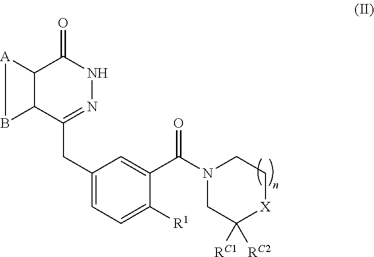
and isomers, salts, solvates, chemically protected forms, and prodrugs thereof wherein:
A and B together represent an optionally substituted, fused aromatic ring;
X is NR^{X} or CR^{X}R^{Y};
if X=NR^{X} then n is 1 or 2 and if X=CR^{X}R^{Y} then n is 1;
R^{X} is selected from the group consisting of H, optionally substituted C₁₋₂₀ alkyl, C₅₋₂₀ aryl, C₃₋₂₀ heterocyclyl, amido, thioamido, ester, acyl, and sulfonyl groups;
R^{Y} is selected from H, hydroxy, amino;
or R^{X} and R^{Y} may together form a spiro-C₃₋₇ cycloalkyl or heterocyclyl group;
R^{C1} and R^{C2} are independently selected from the group consisting of hydrogen and C₁₋₄ alkyl or when X is CR^{X}R^{Y}, R^{C1}, R^{C2}, R^{X} and R^{Y}, together with the carbon atoms to which they are attached, may form an optionally substituted fused aromatic ring; and
R₁ is selected from H and halo;
and
(b) formula (III) 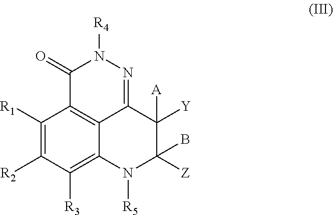
and isomers, salts, solvates, chemically protected forms, and prodrugs thereof wherein:
Y and Z are each independently selected from the group consisting of:
1. an aryl group optionally substituted with 1, 2, or 3 R₆;
2. a heteroaryl group optionally substituted with 1, 2, or 3 R₆;
3. a substituent independently selected from the group consisting of hydrogen, alkenyl, alkoxy, alkoxyalkyl, alkoxycarbonyl, alkoxycarbonylalkyl, alkyl, alkynyl, arylalkyl, cycloalkyl, cycloalkylalkyl, haloalkyl, hydroxyalkylene, oxo, heterocycloalkyl, heterocycloalkylalkyl, alkylcarbonyl, arylcarbonyl, heteroarylcarbonyl alkylsulfonyl, arylsulfonyl, heteroarylsulfonyl, (R_{A}R_{B})alkylene, (NR_{A}R_{B})carbonyl, (NR_{A}R_{B})carbonylalkylene, (NR_{A}R_{B})sulfonyl, and (R_{A}R_{B})sulfonylalkylene;
wherein each R⁶ is selected from OH, NO₂, CN, Br, Cl, F, I, C₁₋₆-alkyl, C₃₋₈-cycloalkyl, C₂₋₈-heterocycloalkyl; C₂₋₆-alkenyl, alkoxy, alkoxyalkyl, alkoxycarbonyl, alkoxycarbonylalkyl, C₂₋₆-alkynyl, aryl, arylalkyl, C₃₋₈-cycloalkylalkyl, haloalkoxy, haloalkyl, hydroxyalkylene, oxo, heteroaryl, heteroarylalkoxy, heteroaryloxy, heteroarylthio, heteroarylalkylthio, heterocycloalkoxy, C₂₋₈-heterocycloalkylthio, heterocyclooxy, heterocyclothio, NR_{A}R_{B}, (R_{A}R_{B})C₁₋₆-alkylene, (NR_{A}R_{B})carbonyl, (R_{A}R_{B})carbonylalkylene, (NR_{A}R_{B})sulfonyl, and (NR_{A}R_{B})sulfonylalkylene;
R₁, R₂, and R³ are each independently selected from the group consisting of hydrogen, halogen, alkenyl, alkoxy, alkoxycarbonyl, alkyl, cycloalkyl, alkynyl, cyano, haloalkoxy, haloalkyl, hydroxyl, hydroxyalkylene, nitro, NR_{A}R_{B}, NR_{A}R_{B} alkylene, and (R_{A}R_{B})carbonyl;
A and B are each independently selected from hydrogen, Br, Cl, F, I, OH, C₁₋₆-alkyl, C₃₋₈-cycloalkyl, alkoxy, alkoxyalkyl wherein C₁₋₆-alkyl, C₃₋₈-cycloalkyl, alkoxy, alkoxyalkyl are optionally substituted with at least one substituent selected from OH, NO₂, CN, Br, Cl, F, I, C₁₋₆-alkyl, and C₃₋₈-cycloalkyl, wherein B is not OH;
R_{A}, and R^{B} are independently selected from the group consisting of hydrogen, alkyl, cycloalkyl, and alkylcarbonyl; or R^{A} and R_{B} taken together with the atom to which they are attached form a 3-10 membered heterocycle ring optionally having one to three heteroatoms or hetero functionalities selected from the group consisting
of —O—, —NH, —N(C₁₋₆-alkyl)-, —NCO(C₁₋₆-alkyl)-, —N(aryl)-, —N(aryl-C₁₋₆-alkyl-), —N(substituted-aryl-C₁₋₆-alkyl-)—, —N(heteroaryl)-, —N(heteroaryl-C₁-C₆-alkyl-)-, —N(substituted-heteroaryl-C₁₋₆ alkyl-)—, and —S— or S(O)q-, wherein q is 1 or 2 and the 3-10 membered heterocycle ring is optionally substituted with one or more substituents;
R₄ and R₅ are each independently selected from the group consisting of hydrogen, alkyl, cycloalkyl, alkoxyalkyl, haloalkyl, hydroxyalkylene, and (NR_{A}R_{B})alkylene; and isomers, salts, solvates, chemically protected forms, and prodrugs thereof;
(iii) that inhibits PARP1 and PARP2 is selected from the group consisting of a compound of
(a) formula (IV) 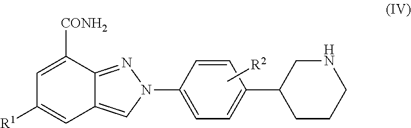
and isomers, salts, solvates, chemically protected forms, and prodrugs thereof wherein:
R₁ is hydrogen or fluorine; and
R₂ is hydrogen or fluorine;
and
(b) formula (V) 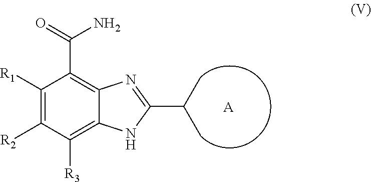
and isomers, salts, solvates, chemically protected forms, and prodrugs thereof wherein:
R₁, R₂, and R³ are independently selected from the group consisting of hydrogen, alkenyl, alkoxy, alkoxycarbonyl, alkyl, alkynyl, cyano, haloalkoxy, haloalkyl, halogen, hydroxy, hydroxyalkyl, nitro, NR_{A}R_{B}, and (NR_{A}R_{B})carbonyl;
A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, heterocycle, heterocyclealkyl, heteroaryl, heteroarylalkyl, hydroxy, hydroxyalkyl, nitro, NR_{C}R_{D}, (NR_{C}R_{D})alkyl, (NR_{C}R_{D})carbonyl, (NR_{C}R_{D})carbonylalkyl, and (NR_{C}R_{D})sulfonyl; and
R_{A}, R_{B}, R_{C}, and R_{D} are independently selected from the group consisting of hydrogen, alkyl, and alkycarbonyl
(iii) that inhibits PARP1, PARP2 and PARP3 is a compound of formula (VI) 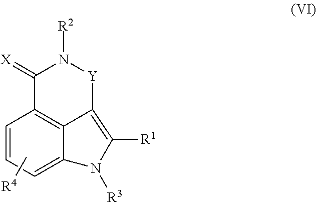
and isomers, salts, solvates, chemically protected forms, and prodrugs thereof wherein:
R₁ is: H; halogen; cyano; an optionally substituted alkyl, alkenyl, alkynyl, cycloalkyl, heterocycloalkyl, aryl, or heteroaryl group (e.g., unsubstituted or substituted with one or more substituents selected from halogen, hydroxy, nitro, and amino, alkoxy, alkyl, and aryl groups unsubstituted or substituted with one or more substituents selected from halogen, hydroxy, nitro, carboxy, and optionally substituted amino and ether groups (such as O-aryl)); or —C(O)—R₁₀, where R₁₀ is: H; an optionally substituted alkyl, alkenyl, alkynyl, cycloalkyl, heterocycloalkyl, aryl, or heteroaryl group (e.g., unsubstituted or substituted with one or more substituents selected from halogen, hydroxy, nitro, amino, and alkyl and aryl groups unsubstituted or substituted with one or more substituents selected from halo, hydroxy, nitro, and amino); or OR₁₀₀ or NR₁₀₀R₁₁₀, where R₁₀₀ and R₁₁₀ are each independently H or an optionally substituted alkyl, alkenyl, alkynyl, cycloalkyl, heterocycloalkyl, aryl, or heteroaryl group (e.g., unsubstituted or substituted with one or more substituents selected from alkyl, alkenyl, alkynyl, cycloalkyl, heterocycloalkyl, aryl, and heteroaryl groups unsubstituted or substituted with one or more substituents selected from halogen, hydroxy, nitro, amino, and alkyl and aryl groups unsubstituted or substituted with one or more substituents selected from halogen, hydroxy, nitro, and optionally substituted amino groups);
R₂ is H or alkyl;
R₃ is H or alkyl;
R₄ is H, halogen or alkyl;
X is O or S;
Y is (CR₅R₆)(CR₇R₈) n or N—C(R₅), where:
n is 0 or 1;
R⁵ and R⁶ are each independently H or an optionally substituted alkyl, alkenyl, alkynyl, cycloalkyl, heterocycloalkyl, aryl, or heteroaryl group (e.g., unsubstituted or substituted with one or more substituents selected from halogen, hydroxy, nitro, amino, and lower alkyl, lower alkoxy, or aryl groups unsubstituted or substituted with one or more substituents selected from halogen, hydroxy, nitro, and amino); and
R₇ and R₅ are each independently H or an optionally substituted alkyl, alkenyl, alkynyl, cycloalkyl, heterocycloalkyl, aryl, or heteroaryl group (e.g., unsubstituted or substituted with one or more substituents selected from halogen, hydroxy, nitro, amino, and lower alkyl, lower alkoxy, and aryl groups unsubstituted or substituted with one or more substituents selected from halogen, hydroxy, nitro, and amino);
where when R₁, R₄, R⁵, R₆, and R⁷ are each H, R₈ is not unsubstituted phenyl.

21. The method of claim 20, wherein the PARPi is selected from the group consisting of Olaparib, Talazoparib, Niraparib, Rucaparib, and Veliparib, in particular of Veliparib, Olaparib, and Talazoparib.

22. The method of claim 18, wherein the proliferative disease is selected from a HRD tumor, a BRCA-1 and/or BRCA-2-deficient tumor, and a tumor in which expression of PARP-1, PARP-2, PARP-3 and/or ALC1 is increased in comparison to non-tumor cells, and/or wherein the proliferative disease is selected from hepatocellular carcinoma, breast cancer, ovarian cancer, prostate cancer, colorectal cancer.

23. The method of claim 19, wherein the PARPi and ALC1i are administered concomitantly or separately.

24. A kit of parts comprising separately packaged a PARPi and the ALC1i of claim 16; or a composition comprising a PARPi and the ALC1i of claim 16.

25. The allosteric inhibitor of ALC1 of claim 16 having the structure: 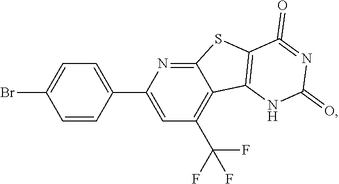
and isomers, salts, solvates, chemically protected forms, and prodrugs thereof.

26. The allosteric inhibitor of ALC1 of claim 16 having the structure: 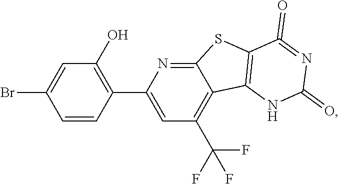
and isomers, salts, solvates, chemically protected forms, and prodrugs thereof.
